(19) United States
(12) Patent Application Publication
(10) Pub. No.: US 2011/0044968 A1
(43) Pub. Date: Feb. 24, 2011

(54) COMPOSITIONS FOR TREATMENT WITH METALLOPEPTIDASES, METHODS OF MAKING AND USING THE SAME

(75) Inventors: Elijah M. Bolotin, Bothell, WA (US); Gerardo Castillo, Bothell, WA (US); Penelope Markham, Clifton, VA (US); Manshun Lai, Bothell, WA (US)

Correspondence Address: WILSON SONSINI GOODRICH AND ROSATI / PHARMAIN LTD, 650 PAGE MILL ROAD, PALO ALTO, CA, 94304, US

(73) Assignee: Pharmal N Corporation, Seattle, WA (US)

(21) Appl. No.: 12/921,670

(22) PCT Filed: Mar. 10, 2009
(86) PCT No.: PCT/US09/36648

§ 371 Date: Nov. 5, 2010

Related U.S. Application Data
(60) Provisional application No. 61/068,896, filed on Mar. 10, 2008.

(51) Int. Cl. A61K 38/54; C12N 9/96; A61P 31/00; A61P 25/28

(52) U.S. Cl. 424/94.3; 435/188

(57) ABSTRACT

The present invention is directed to biocompatible compositions and the use of metal bridges to connect a back-bone and a metallopeptidase active agent. In certain instances, the subject compositions provide a means of achieving sustained release of the metallopeptidase active agent after administration to a subject.

### CROSS-REFERENCE

This application claims the benefit of U.S. Provisional Application No. 61/068,896 filed Mar. 10, 2008, which application is incorporated herein by reference in its entirety.

### STATEMENT AS TO FEDERALLY SPONSORED RESEARCH

This invention was made with the support of the U.S. Government under Grant No. 5R43AI078539 awarded by the National Institute of Allergy and Infectious Disease (NIAID). The U.S. Government may have certain rights to the subject matter provided herein.

### BACKGROUND OF THE INVENTION

The development of new drugs, formulations and other systems for administration of physiologically active peptides and proteins and other therapeutics and materials is driven by the need to provide these peptides or proteins or other materials to achieve the desirable physiological effects. With respect to peptides and proteins, many of them have been observed to be unstable in the gastro-intestinal tract and therefore may need to be stabilized or protected or delivered via systemic circulation. In addition, peptides and proteins that have low molecular masses tend to have short biological half-lives due to their efficient removal from systemic circulation via kidneys and reticuloendothelial system. Many peptides and proteins can also lose their activity in vivo due to proteolysis (peptide bond cleavage).

In part to circumvent these undesirable effects, a drug delivery system may be used. Drug delivery strategies have been developed for peptide and protein delivery in vivo, but most are not useful for sustained delivery. For example, the use of a continuous systemic infusion of drug via a pump is impractical for outpatients requiring high levels of mobility and has the associated disadvantages of quality of life and potential intravenous (I.V.) line infections. The use of an implantable pump, comprised of a capsule with a membrane allowing diffusion of a drug, is limited by the volume of the capsule. Peptides and proteins are often used in concentrated formulations in the capsules and aggregate, whereby losing specific activity. In many cases, the drug is released into the extracellular space and distributed in lymphatics. Other implantable biodegradable delivery systems are implanted or injected into the epidermis. The components of the system are usually slowly degraded as a result of biological activity of surrounding cells (i.e. as a result of the release of enzymes degrading chemical bonds that hold these implants together).

Metallopeptidases, interchangeably known as metalloproteinases and metalloproteases, encompass a large family of enzymes sharing the common feature of containing a metal in the active site. The use of metallopeptidases has a lot of therapeutic potential, including uses in treating cancer and related neoplastic diseases, systemic infections, and diseases of the nervous system such as Alzheimer's disease. There is a need for a biodegradable drug delivery carrier for the systemic delivery of metallopeptidases that would result in longer circulation in the body, more stability in the blood, and can be more conveniently administered.

### SUMMARY OF THE INVENTION

In part, the present invention is directed to biocompatible compositions and use of metal bridges to connect a backbone and a metallopeptidase active agent. In certain instances, the subject compositions provide a means of achieving sustained release of the metallopeptidase active agent after administration to a subject. The metallopeptidase can be one that is selected from those listed in Table 1 or Table 2. In one embodiment, the metallopeptidase active agent is a metalloexopeptidase or a metallocarboxypeptidase. In another embodiment, the metallopeptidase active agent is a metalloendopeptidase. In a specific embodiment, the metallopeptidase active agent is a glycyl-glycyl metalloendopeptidase, such as lysostaphin. In another specific embodiment, the metallopeptidase active agent is the metalloendopeptidase neprilysin.

In one aspect, the present invention relates composition containing (i) a polymeric backbone with monomeric units, (ii) a chelating group covalently linked to a monomeric unit, (iii) a transition metal ion and iv) a metallopeptidase active agent coordinately bonded to the transition metal ion. In another aspect, the present invention relates to a composition comprising (i) an aliphatic group, (ii) a chelating group covalently linked to the aliphatic group, (iii) a transition metal ion, and iv) a metallopeptidase active agent coordinately bonded to the transition metal ion.

The polymeric backbone of the subject compositions can be chosen from but not limited to polylysine, polyaspartic acid, polyglutamic acid, polyserine, polythreonine, polycysteine, polyglycerol, polyethyleneimines, polyallylamine, chitosan, natural saccharides, aminated polysaccharides, aminated oligosaccharides, polyamidoamine, polyacrylic acids, polyalcohols, sulfonated polysaccharides, sulfonated oligosaccharides, carboxylated polysaccharides, carboxylated oligosaccharides, aminocarboxylated polysaccharides, aminocarboxylated oligosaccharides, carboxymethylated polysaccharides, or carboxymethylated oligosaccharides.

The aliphatic chain of the subject compositions can be within a general formula [PvNwCxHyOz-] where v is 0-3, w is 0-3, x is 8-48; y is 15-95; z is 1-13. In a further embodiment of the above compositions with an aliphatic group, the aliphatic group is an alkyl group. In one embodiment the aliphatic chain comprises from C8 to C36 carbon atoms inclusive. In a further embodiment, the alkyl group comprises a general formula [CH₃(CH)x-] where x is 5-35. In a further embodiment, the aliphatic group comprises one or more alkyl group(s) derived from various fatty acids or fatty acids with aromatic group(s). In further embodiments, the aliphatic group is within the structure that comprises phospholipids or derivative of phospholipids. In further embodiments, the aliphatic group is within the structure that comprises diacylglycerol or derivatives of diacylglycerol. In a further embodiment, the alkyl group comprises a branched alkyl group. In a further embodiment, the alkyl group has one or more double bonds. In a further embodiment, the alkyl group is an ethyl, or propyl group. In a further embodiment, the alkyl group is a butyl, or pentyl group.

In further embodiments of the above compositions with polymeric or aliphatic backbones comprising hydrophobic groups, the hydrophobic groups can be but not limited to, poly-L-glycine, poly-L-alanine, poly-L-valine, poly-L-leucine, poly-L-isoleucine, poly-L-phenylalanine, poly-L-proline, poly-L-methionine, poly-D-glycine, poly-D-alanine, poly-D-valine, poly-D-leucine, poly-D-isoleucine, poly-D-phenylalanine, poly-D-proline, poly-D-methionine, poly-D/L-glycine, poly-D/L-alanine, poly-D/L-valine, poly-D/L-leucine, poly-D/L-isoleucine, and poly-D/L-phenylalanine, poly-D/L-proline, poly-D/L-methionine, phenyl, naphthyl, cholesterol, vitamin D, and/or vitamin E.

The chelating groups of the above compositions can be selected from but are not limited to a nitrogen-containing polycarboxylic acid, a polypeptide having the formula (AxHy)p, wherein A is any amino acid residue, H is histidine, x is an integer from 0-6; y is an integer from 1-6; and p is an integer from 2-6, or more specifically a trimethyl-1,4,7-triazacyclononane; 1,4,7,10-tetraazacyclododecane-N,N′,N″,N′″-tetraacetic acid; 1,4,7,10-tetraaza-cyclododecane-N,N′,N″-triacetic acid; 1,4,7-tris(carboxymethyl)-10-(2′-hydroxypropyl)-1,4,7,10-tetraazocyclodecane; 1,4,7-triazacyclonane-N,N′,N″-triacetic acid; 1,4,8,11-tetraazacyclotetra-decane-N,N′,N″,N′″-tetraacetic acid; 1,2-diaminocyclohexane-N,N,N′,N′-tetraacetic acid; bis(aminoethanethiol)carboxylic acid; diethylenetriamine-pentaacetic acid (DTPA); ethylenediamine-tetraacetic acid (EDTA); ethyleneglycoltetraacetic acid (EGTA); ethylene-bis(oxyethylene-nitrilo)tetraacetic acid; ethylenedicysteine; Imidodiacetic acid (IDA); N-(hydroxyethyl)ethylenediaminetriacetic acid; nitrilotriacetic acid (NTA); nitrilodiacetic acid (NDA); triethylenetetraamine-hexaacetic acid (TTHA); a nitrogen-containing polycarboxylic acid, or a bisphosphonate such as pamidronate, etidronate, alendronate, ibandronate, zoledronate, risendronate or derivatives thereof.

The metal ions used in the compositions of the present invention can be Zn²⁺, Ni²⁺, Co²⁺, Fe²⁺, Mn²⁺, or Cu²⁺. In specific embodiments, the ion is Zn²⁺ and in other specific embodiments the ion is Ni²⁺.

In certain embodiments, the compositions of the present invention comprising polymeric or aliphatic backbones further comprise protective side chains covalently bonded to the backbones. These protective side chains include but are not limited to poly(ethyleneglycol), alkoxy poly(ethylene glycol) and methoxy poly(ethyleneglycol).

The present invention also provides for pharmaceutical compositions comprising either a polymeric backbone or an aliphatic backbone further comprising a chelating group covalently bonded to the backbone, a transition metal ion chelated to the chelating group, a protective chain covalently bonded to the backbone, a metallopeptidase such as lysostaphin or neprilysin coordinately bonded to the transition metal ion, and a pharmaceutically acceptable excipient. In one exemplary embodiment the backbone is polylysine, the chelating agent is NTA, the metal ion is Zn or Ni, the protective chain is MPEG, and the metallopeptidase is lysostaphin in combination with a pharmaceutically acceptable excipient. This composition can be used for the treatment of systemic or other infections in a subject, preferably human.

The pharmaceutical compositions can further comprise an antibiotic selected from but not limited to Amoxicillin, Ampicillin, Azidocillin, Azlocillin, Aztreonam, Bacitacin, Benzathine benzylpenicillin, Benzathine phenoxymethylpenicillin, Benzylpenicillin(G), Biapenem, Carbenicillin, Cefacetrile, Cefadroxil, Cefalexin, Cefaloglycin, Cefalonium, Cefaloridine, Cefalotin, Cefapirin, Cefatrizine, Cefazedone, Cefazaflur, Cefazolin, Cefradine, Cefroxadine, Ceftezole, Cefaclor, Cefamandole, Cefminox, Cefonicid, Ceforanide, Cefotiam, Cefprozil, Cethuperazone, Cefuroxime, Cefuzonam, cephamycin (such as Cefoxitin, Cefotetan, Cefinetazole), carbacephem (such as Loracarbef), Cefcapene, Cefdaloxime, Cefdinir, Cefditoren, Cefetamet, Cefixime, Cefinenoxime, Cefodizime, Cefoperazone, Cefotaxime, Cefpimizole, Cefpiramide, Cefpodoxime, Cefsulodin, Ceftazidime, Cefteram, Ceftibuten, Ceftiolene, Ceftizoxime, Ceftriaxone, oxacephem (such as Flomoxef, Latamoxef), Cefepime, Cefozopran, Cefpirome, Cefquinome, Ceftobiprole, Chloroamphenicol, Chlorohexidine, Clindamycin, Clometocillin, Cloxacillin, Colistin, Cycloserine, Daptomycin, Doripenem, Doxycycline, Epicillin, Ertapenem, Erythromycin, Faropenem, Fostomycin, Gentamycin, Imipenem, Linezolid, Mecillinam, Meropenem, Methicillin, Meticillin, Mezlocillin, Minocycline, Mupirocin, Nafcillin, Neomycin, Oxacillin, Panipenem, Penamecillin, Pheneticillin, Phenoxymethylpenicillin (V), Piperacillin, Polymyxin, Polymyxin B, Procaine benzylpenicillin, Propicillin, Quinupristin/dalfopristin, Ramoplanin, Rifampicin, Rifampin, Sulbenicillin, Teicoplanin, Tigecycline, Tigemonam, Trimethoprim/sulfamethoxazole, and Vancomycin.

The present invention provides a number of methods of making and using the subject compositions. Examples of such methods include those described herein in the Examples.

The present invention provides method of administering the pharmaceutical compositions described herein to a subject. Such methods are provided can be for treating a subject diagnosed with or suspected of having or developing an infection, a neoplastic disease or a nervous system disease such as Alzheimer's Disease.

In another embodiment, the present invention relates to a method of treatment, comprising administering any of the above described compositions. In a further embodiment, the present invention relates to a method of treating primary bloodstream infections, bacterial infective endocarditis (KE), osteomyelitis, infections involving bacterial biofilms, or community-acquired MRSA infections comprising administering any of the above described compositions.

In another embodiment, the present invention relates to a kit comprising a composition comprising: (i) a polymeric or aliphatic backbone (ii) a chelating moiety covalently linked or bonded to the backbone; (iii) a metal ion chelated to the chelating moiety by at least two coordinate bonds; (iv) a metallopeptidase active agent with a metal binding domain (MBD, which may or may not be a chelator) coordinately bonded to the metal ion; and optionally (v) a protective chain covalently linked or bonded to the backbone. Uses for such kits include, for example, therapeutic applications. Such kits may have a variety of uses, including, for example, imaging, targeting, diagnosis, therapy, vaccination, and other applications.

In another aspect, the compositions of the present invention may be used in the manufacture of a medicament for any number of uses, including for example treating any disease or other treatable condition of a patient. In still other aspects, the present invention is directed to a method for formulating biocompatible compositions of the present invention in a pharmaceutically acceptable excipient.

These embodiments of the present invention, other embodiments, and their features and characteristics, will be apparent from the description, drawings and claims that follow.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 depicts a metallopeptidase metal ion bridge composition. This exemplary diagram depicts a polymeric backbone with protective chains, attached to a chelating moiety which is chelated to a metal ion, which in turn coordinates a metal containing metallopeptidase.

FIG. 2 shows the percent of an exemplary glycyl-glycyl metalloendopeptidase, lysostaphin, bound to a carrier linked to NTA-Zn (lot#20020105) after incubation for 1 hr at room temperature. However in the absence of Zn, no lysostaphin was found to be associated with the carrier indicating that Zn is necessary for complex formation.

These data demonstrate that it is feasible to formulate a metallopeptidase such as lysostaphin with a carrier linked to NTA-Zn with a loading capacity of at least 6 lysostaphin molecules per molecule of carrier. The carrier used is a PLPEG with 40 kDa polylysine-Br with 33% of the epsilon amino groups saturated with MPEG and the rest of the lysine residues were modified by NTA derived from Nalpha,Nalpha-)biscarboxymethyl) lysine.

FIGS. 3-5 show the Scatchard plots (y-axis is bound/free; x-axis is bound; slope is −1/kd; x-intercept is the capacity) with various Kds and capacity of three selected carriers for demonstration purpose. Example 19 details the procedure to obtain these data.

In FIG. 3, the carrier named is 20PLPEG550DAPEI4NTAZn (lot#20080603c), a regression line equation of y=−6.43x+79.94 with a corresponding capacity of 19 lysostaphin molecules per carrier and a Kd of 156 nM.

FIG. 4, the carrier named is 20PLPEG550DAPEI8NTAZn (lot#20080604c), a regression line equation of y=−8.68x+111.6 with a corresponding capacity of 20 lysostaphin molecules per carrier and a Kd of 115 nM.

In FIG. 5, the carrier named is 20PLPEG550DAPEI12NTAZn (lot#20080605c) with a regression line equation of y=−10.09x+152.5, a corresponding capacity of 24 lysostaphin molecules per carrier and a Kd of 99 nM.

FIG. 6 shows the level activity of lysostaphin in rat serum with time. Sprague-Dawley Rats (n=5; 250-350 g) were given an intravenous injection of lysostaphin alone or lysostaphin formulated in carrier lot #20080421a, 20080326, and 20080421a at 50% loading (i.e. weight of lysostaphin is 50% of the weight of the carrier). Both lysostaphin alone and the formulations were dissolved in saline. Blood samples were collected from the tails at various time points in tubes containing a protease inhibitor cocktail. Serum was collected from each sample by centrifugation using a clinical centrifuge and lysostaphin activity was assayed.

FIG. 7 shows the level activity of lysostaphin in rat serum with time. Sprague-Dawley Rats (n=5; 250-350 g) were given an intravenous injection lysostaphin alone or lysostaphin formulated in carrier lot #20080603c and 20080804b at 50% loading (i.e. weight of lysostaphin is 50% of the weight of the carrier). Both lysostaphin alone and the formulations were dissolved in saline. Blood samples were collected from the tails at various time points in tubes containing a protease inhibitor cocktail. Serum was collected from each sample by centrifugation using a clinical centrifuge and lysostaphin activity was assayed.

FIG. 8 shows the level activity of lysostaphin in rat serum with time. Sprague-Dawley Rats (n=5; 250-350 g) were given an intravenous injection lysostaphin alone or lysostaphin formulated in carrier lot #20080603c, 20080604c, and 20080605c at 20% loading (i.e. weight of lysostaphin is 20% of the weight of the carrier). Both lysostaphin alone and the formulations were dissolved in saline. Blood samples were collected from the tails at various time points in tubes containing a protease inhibitor cocktail. Serum was collected from each sample by centrifugation using a clinical centrifuge and lysostaphin activity was assayed.

FIG. 9 depicts a graph showing the binding of human growth hormone (hrGH) to polymers in the presence of Zn and Ni cations. Size-separation on Centricon YM-100 membrane suggests that approximately 1 mg of rhGH binds to 100 mg of PLPEGNTAZn (lot#20020105). This result is presented to demonstrate that the presence of a chelated metal attached to the backbone is essential for binding of a protein with a metal binding domain or known to have the ability to bind metals.

FIG. 10 depicts a chromatogram showing elution profiles of ¹²⁵I-labled rhGH (squares) and rhGH complex with PLPEGNTAZn (circles) on SEC-5 size-exclusion HPLC column. The profile of time-dependent elution shows that a fraction of the complex of labeled hormone with PLPEGNTAZn (lot#20020105) elutes earlier than the free hormone suggesting a complex formation. The rhGH is dragged to the void volume by the carrier containing metal chelate. This result is presented to demonstrate that the interaction of chelated metal in the metal binding domain of the protein is stable and can survive the gel permeation chromatography involving thousands of re-equilibration (equal to the number of theoretical plates of the column) as the sample passes through the column. Weak interaction will cause the complex to dissociate resulting in an unaltered rhGH peak which is not observed in this case.

FIG. 11 depicts a bar-graph showing histidine tagged-GFP binding yields after separation of complexes with PLPEGNTA(Ni or Zn salts), PLPEG(lot#20020101) or PLPEGSA(lot#20020102) in the presence or absence of blood plasma. The graph shows that complex formation with metal salts of PLPEGNTA (lot#20020103) is equally possible in the presence or absence of bulk protein of plasma. The same is behavior is expected if a metallopeptidase is altered by addition of histidine tag (a metallopeptidase derivative).

FIG. 12 depicts a bar graph showing the levels of GFP in plasma of animals injected with a histidine tagged-GFP (control); and complexes of histidine tagged-GFP with PLPEGNTAZn (lot#20020105) and PLPEGNTANi (lot#20020104). The graph shows significantly higher in vivo levels of GFP in blood in the case of the Ni-complex suggesting prolonged circulation of histidine tagged-GFP bound to PLPEGNTANi carrier. This shows that if a metallopeptidase is altered in a similar manner, a similar improvement is expected.

FIG. 13 depicts a carrier targeting inflammation and infection sites. Carriers of the present invention have long-circulation and can efficiently accumulate in sites of E. coli-induced inflammation and thus represent an alternative to inflammation-specific agents. For this experiment, male Sprague-Dawley rats infected with previously frozen Escherichia coli (diluted in sterile isotonic saline to a final viable cell titer of 9×108 organisms per 0.15 mL) in the posterior portions of the left thigh muscle. 3D maximum intensity projection MR images at 1, 12 and 24 hours after IV administration of gadolinium-labeled PLPEGDTPA.

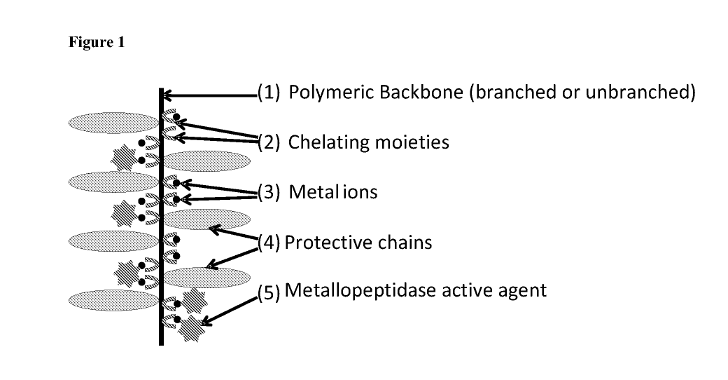

### DETAILED DESCRIPTION OF THE INVENTION

### Definitions

For convenience, before further description of the present invention, certain terms employed in the specification, examples and appended claims which need further explanations are collected here. These definitions should be read in light of the remainder of the disclosure and understood as by a person of skill in the art. Unless defined otherwise, all technical and scientific terms used herein have the same meaning as commonly understood by a person of ordinary skill in the art.

The articles “a” and “an” are used to refer to one or to more than one (i.e., to at least one) of the grammatical object of the article. By way of example, “an element” means one element or more than one element.

The term “derivative” or “analog” as used herein includes compounds whose core structures are the same as, or closely resemble that of, a parent compound, but which have a chemical or physical modification, such as different or additional groups; the term includes co-polymers of parent compounds that can be linked to other atoms or molecules. The term also includes a peptide or protein with at least 50% sequence identity with the parent peptide or protein. The term also includes a peptide with additional groups attached to it, such as oligonucleotides and/or additional amino acids, compared to the parent peptide. The term also includes a polymer with additional group attached to it, such as alkoxy group, compared to the parent polymer.

The term “naturally-occurring” or “native”, includes objects that may be found in nature. For example, a backbone that may be isolated from a source in nature and which has not been intentionally modified, for example, in the laboratory, is naturally-occurring. The term “non-naturally-occurring” or “non-native” or “synthetic” is as applied to an object that has been intentionally modified for example, in the laboratory, and not normally found in nature.

### General Introduction

Embodiments of the present invention are directed at carrier-based metallopeptidase delivery systems comprising of a backbone, a metal binding domain covalently linked to the backbone, a metal ion chelated by the metal binding domain, and a metallopeptidase coordinately bonded to the metal ion. Optionally, the backbone can contain multiple protective chains to shield or protect the metallopeptidase. Protective chains can increase the overall hydrodynamic radius of the macromolecular agent which can result in prolonged circulation in the blood and increase accumulation at sites of high vascular permeability.

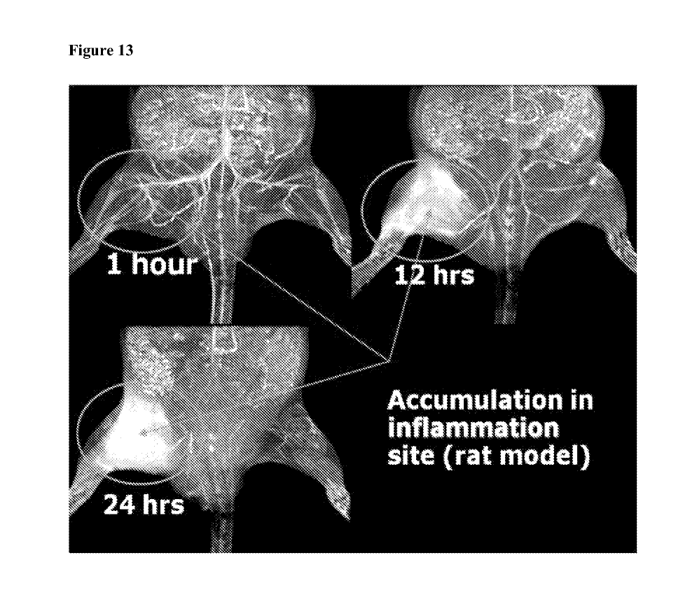

The carriers of the present invention can permeate broken down or abnormal vascular barriers due to their high permeability levels. This is demonstrated in a model of bacterial inflammation of the muscle tissue in rats induced with E. coli. The carrier could be used for early detection of leakage into the extra vascular space and specific targeting to the sites with increased vascular permeability, such as inflammation (see FIG. 13). Thus, increased accumulation of the carrier at sites of inflammation will allow the carrier-associated-metallopeptidase to accumulate at sites of infection.

The association of a metallopeptidase or a derivate thereof to the backbone is accomplished using a metal bridge. The use of metallopeptidase derivatives can maintain or enhance metal coordination ability. Examples of metallopeptidase derivatives are His-Tagged metallopeptidases. An advantage of chelating metals to the carriers of the present invention is to afford reversible binding of metallopeptidases which are capable of forming coordination bonds with metal ions (e.g., Zn, Cu, or Ni). The coordinate bonding affords reversible dissociation of metal binding metallopeptidase active agents from the backbone containing the chelated metal.

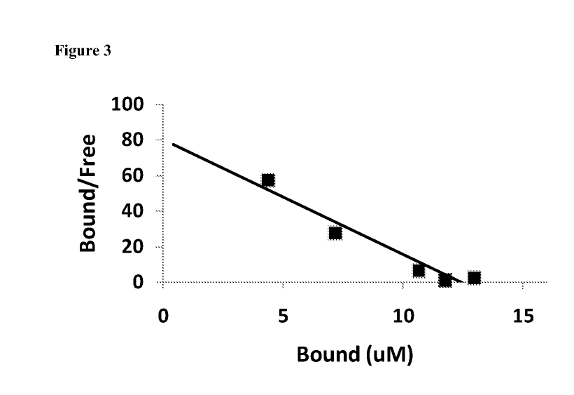

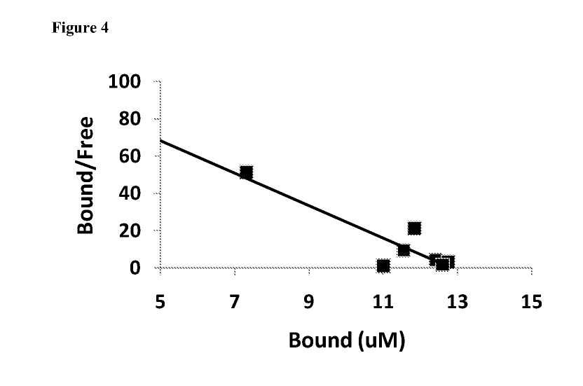

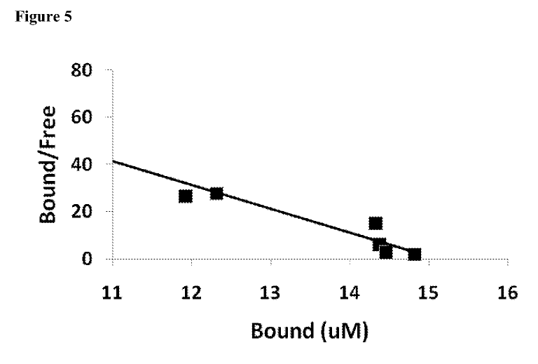

The carrier-chelated-metal-metallopeptidase formulation can provide several benefits. For example such formulations afford better biocompatibility; decrease potential toxicity; decrease immunogenicity; increase blood residence time; enable site-specific accumulation at sites of inflammation (for example, see FIG. 13). The carriers of the present invention have high drug loading capacities as well; for example, see FIGS. 3-5 with the specific reversible binding of an exemplary metalloendopeptidase, lysostaphin.

Based on results presented, metallopeptidases bind to the chelating moiety of the carrier metal coordination. The metal coordination can be of one or more histidines in addition to other amino acids. Interactions may also be facilitated by interactions with protective chains and/or other components of the carrier. The design of the carriers of the present invention is made in such a way that the associated metallopeptidases are protected by the protective chains (for example polyethylene glycol chains) from for example peptidases and antibodies. In addition, the association of metallopeptidases such as lysostaphin with the high molecular weight carrier can prolong its half life by preventing its excretion via renal ultrafiltration, uptake by antigen presenting cells, and uptake by reticuloendothelial system.

### Components of the Carrier of the Invention and Metallopeptidases

The carriers of the present invention are comprised of a backbone that may be polymers/co-polymers or aliphatic chains capable of supporting at least one chelator which in turn chelates a metal ion which in turn coordinates a metallopeptidase. In further embodiments of the present invention, the backbone further comprises a protective chain covalently linked to the backbone. In one aspect, the carrier is biocompatible. The individual components are described below.

A) Backbones

The backbones of the carriers of the present invention can be polymers and co-polymers of linear or branched structure or conjugates thereof. Alternatively, backbones of the carriers of the present invention can be aliphatic chains of linear or branched structure or conjugates thereof.

1) Polymeric or Co-polymeric Backbones:

Polymers are composed of repeating structural units connected by covalent chemical bonds. A co-polymer is a polymer derived from two or more different structural units.

In certain embodiments, the backbone polymers or backbone co-polymers of the subject compositions have molecular weights ranging from about 500 to 10,000, 20,000, 30,000, 40,000, or 50,000, 60,000, 70,000, 80,000, 90,000, or 100,000 Daltons and even more specifically between 5,000 to 50,000 Daltons. The number-average molecular weights (Mn) may also vary widely, but generally fall in the range of about 1,000 to about 120,000 Daltons, or even from about 5,000 to about 70,000 Daltons or even from about 10,000 to about 50,000 Daltons. In certain embodiments, the Mn varies between about 8,000 and 45,000 Daltons. Within a given sample of a subject polymeric backbone, a wide range of molecular weights may be present. For example, molecules within the sample may have molecular weights which differ by a factor of 2, 5, 10, 20, 50, 100, or more, or which differ from the average molecular weight by a factor of 2, 5, 10, 20, 50, 100, or more. The number of monomers in the backbone polymer may vary from 10 (a 10-mer) to 1,000 (a 1,000-mer). The backbone polymer may alternatively be about a 25, 50, 100, 150, 200, 250, 300, 350, 400, or 450-mer, and even more specifically between a 100-mer to 250-mer. The number of monomers in the polymeric backbone generally determines the number of functional groups that can be modified to carry chelating moieties or protective chains.

In some embodiments, the polymeric backbone can be a non-proteinaceous homo- or heteropolymer with repeating monomeric groups containing amino, carboxyl, hydroxyl, thiol, sulfate, or phosphate groups and may be of natural or synthetic origin, wherein the repeating monomeric groups can be covalently modified to contain chelating groups and optionally hydrophilic protective chains. In other embodiments the polymeric backbone may also be a non-proteinaceous homo- or heteropolymer but rather contain repeating hydrophobic groups with terminal amino, carboxyl, hydroxyl, thiol, sulfate, phosphate groups or any modifiable functional groups that can be covalently modified to contain a chelating group and optionally hydrophilic protective chains. The term “non-proteinaceous polyamino acid” as used herein includes a polyaminoacid that is not naturally made by a living organism unless recombinantly engineered or does not have enzymatic or biological activity resulting from its three dimensional conformation. In certain embodiments, the polymeric backbone is a polyamino acid which may have D- or L-chirality or both and is a straight chain homopolymer. In one specific embodiment, straight chain homopolymers include polylysine and polyornithine, polyarginine, polyglutamate, polyaspartate, polyserine, polythreonine, polytyrosine or any other amide linked homopolymer made from amino acids. In another preferred embodiment, straight chain hydrophobic homopolymers comprise polyalanine, polyvaline, polyleucine, polyisoleucine, polyglycine, or polyphenylalanine. These hydrophobic polyamino acids can be modified at one terminal to contain chelating groups and at the other terminal to contain hydrophilic protective chains. If the backbone is a polymer comprising polyamino acids, it is usually non-proteinaceous, meaning that it is not a naturally occurring protein with activity associated with its three dimensional conformation. The polymeric backbone may have a molecular weight of about 600-1,000,000 daltons, preferably 10,000-100,000 daltons. Other polymeric backbones with repeating modifiable functional groups may also be used such as those with repeating sulfhydryl(thiol), phosphate, and hydroxyl groups. Carbohydrate polymers and other synthetic polymers where monomers are non-biological may also be used as the polymeric backbone. The polymeric backbone provides multiple sites from where the chelating groups and hydrophilic protective chains can be attached.

Polymeric backbones can include polysaccharides. Polysaccharides encompass disaccharides, oligosaccharides and larger polymers of up to millions of Daltons. Polymeric backbones include polysaccharides, oligosaccharides and products chemically derived thereof, bearing modifiable carboxylic groups, alcohol groups or amino groups, which may be exemplified by: polyxylotol, galacturonic acid, glucuronic acid, mannuronic acid, hyaluronic acid, pectic acid, neuraminic acid, alginic acid, carrageenan; oxidized dextrans; aminated dextran, e.g. containing linked amino groups. Polymeric backbones including polysaccharides may be linear or branched, may be carboxylated, carboxymethylated, sulfated or phosphorylated. Polymeric backbones including polysaccharides can be reacted with derivatives of carbonic, dicarbonic, sulfuric, aminosulfuric, phosphoric acids with resultant linking of carboxylic, aminocarboxylic, carboxymethyl, sulfuric, amino or phosphate groups. Polymeric backbones including polysaccharides can be obtained by chemical alteration of dextran, mannan, xylan, pullulan, cellulose, chytosan, agarose, fucoidan, galactan, arabinan, fructan, fucan, chitin, pustulan, levan or pectin. In addition these polysaccharides may be represented by heteropolymers or homopolymers of monosaccharides such as but not limited to glucose, galactose, mannose, galactose, deoxyglucose, ribose, deoxyribose, arabinose, fucose, xylose, xylulose, and ribulose.

Polymeric backbones also include polymers (linear or branched) such as polyethyleneimine, polyamidoamine, polyallyamine, polyacrylic acid, and polyalcohols (e.g. polyvinylalcohol) to which carboxylic, amino or alcohol groups are chemically linked and/or available for attachment of chelating groups. These polymeric backbones can be non-biological to which carboxylic, amino, or alcohol groups are available for attachment of chelating groups.

In another embodiment, the polymer acting as the polymeric backbone may be poly(ethylene glycol) (PEG) with functional groups at the terminal end or near the terminal end making up the chelating group to which the metal ion coordinates and in turn coordinates the metallopeptidase. Schematically this embodiment may be represented by the following: PEG-chelator-Metal-MBD(metal binding domain)-metallopeptidase. Alternatively, PEG may be functionalized along its backbone allowing chelator-Metal-MBD-metallopeptidase moieties to be pendant to the backbone. This structure may also allow pendant protective chains as well.

2) Aliphatic Backbones

In certain embodiments, the backbone is an aliphatic chain. The term “aliphatic” is art-recognized and includes linear, branched, cyclic alkanes, alkenes, or alkynes. In organic chemistry, compounds composed of carbon and hydrogen are divided into two classes: aromatic compounds, which contain benzene and other similar compounds, and aliphatic compounds (fat, oil), which do not. In aliphatic compounds, carbon atoms can be joined together in straight chains, branched chains, or rings (in which case they are called alicyclic). They can be joined by single bonds (alkanes), double bonds (alkenes), or triple bonds (alkynes). Besides hydrogen, other elements can be bound to the carbon chain, the most common being oxygen, nitrogen, sulfur, and chlorine. The simplest aliphatic compound is methane (CH₄). Aliphatics include alkanes such as fatty acids and paraffin hydrocarbons, alkenes (such as ethylene) and alkynes (such as acetylene). The term “aliphatic” as used herein also includes halosubstituted aliphatics instead of hydrogen. The term “alkyl” is an art-recognized subgroup of “aliphatics”, and includes saturated aliphatic groups, including straight-chain aliphatic groups, branched-chain aliphatic groups, cycloaliphatic (alicyclic) groups, aliphatic substituted cycloaliphatic groups, and cycloaliphatic substituted aliphatic groups. In certain embodiments, a straight chain or branched chain aliphatic has about 36 or fewer carbon atoms in its backbone (e.g., C₁-C₃₆ for straight chain, C₃-C₃₆ for branched chain), and alternatively, about 24 or fewer. Likewise, cycloaliphatics have from about 3 to about 10 carbon atoms in their ring structure, and alternatively about 5, 6 or 7 carbons in the ring structure. In certain embodiments, the aliphatic backbone of the present invention, where the chelating group is covalently linked or bonded, are linear or branched and have from 6 to about 72 carbon atoms. In specific embodiments the aliphatic backbone has 8-36 carbons attached to modifiable functional groups to add the chelating groups. The term aliphatic backbone refers to the aliphatics described above and their modified derivatives such that the aliphatic chains capable of being modified to covalently link chelating groups and optionally protective chains. In one embodiment, the aliphatic backbone is an 18 carbon aliphatic chain modified by oxidation to contain a carboxylic acid at the terminal which makes it a fatty acid, or more specifically a stearic acid. The acid portion can be linked to a chelating moiety such as NTA, IDA or DTPA, thus providing an aliphatic backbone with a chelating moiety covalently linked to the aliphatic backbone. In other embodiments, the oxidation-modified aliphatic backbone ready for further linkage to a chelating group is caprylic acid (C8), Capric acid (C10), Laurie acid (C12), Myristic acid(C14), Palmitic acid(C16), Arichidic acid (C20), Behenic acid (C22), or Lignoceric acid (C24).

In one embodiment, the aliphatic backbone can be within a general formula [PvNwCxHyOz-] where v is 0-3, w is 0-3, x is 8-48; y is 15-95; z is 1-13. In another embodiment, the alkyl group comprises a general formula [CH₃(CH)x-] where x is 5-35. In a further embodiment, the aliphatic group comprises one or more alkyl group(s) derived from various fatty acids or fatty acids with aromatic group(s). In further embodiments, the aliphatic group is within the structure that comprises phospholipids or derivative of phospholipids. In further embodiments, the aliphatic group is within the structure that comprises diacylglycerol or derivatives of diacylglycerol. In a further embodiment, the alkyl group comprises a branched alkyl group. In a further embodiment, the alkyl group has one or more double bonds. In a further embodiment, the alkyl group is an ethyl, or propyl group. In a further embodiment, the alkyl group is a butyl, or pentyl group.

B) Metal Binding Domains

In general, the metal binding domains (MBDs) of the present invention contain a Lewis base moiety or functional group that encompasses numerous chemical moieties having a variety of structural, chemical and other characteristics capable of forming coordination bonds with a metal ion. The types of functional groups capable of forming coordinate complexes with metal ions are too numerous to categorize here, and are known to those of skill in the art. For example, such moieties will generally include functional groups capable of interaction with a metal center, e.g., heteroatoms such as nitrogen, oxygen, sulfur, and phosphorus. It should be noted that chelating groups or moieties are a subgroup of the larger metal binding domain (MBD) group. Thus there are two types of MBDs: a) chelating groups or moieties, and b) non-chelating groups or moieties which are still coordinately bonding with metal. Both types are able to coordinate bond with metals. The nature of coordinate bonding is that metal cations are often Lewis acids and are therefore able to bind various moieties that may serve as Lewis bases. In general, a moiety serving as a Lewis base will be a strongly acidic group prior to proton loss, (e.g., with a pKa less than about 7, and more preferably less than 5). Once a proton is lost, it is a conjugate base that under the appropriate conditions is a strong enough Lewis base to donate an electron pair to a metal ion to form a coordinate bond. The degree of this Lewis acid-to-Lewis base (metal ion-to-metal binding domain) interaction is a function not only of the particular metal ion, but also of the coordinating moiety itself, because the latter may vary in the degree of basicity as well as in size and stearic accessibility. Exemplary Lewis basic moieties which may be included in the metal binding domain include: amines (primary, secondary, and tertiary) and aromatic amines, amino groups, amido groups, nitro groups, nitroso groups, amino alcohols, nitriles, imino groups, isonitriles, cyanates, isocyanates, phosphates, phosphonates, phosphites, phosphines, phosphine oxides, phosphorothioates, phosphoramidates, phosphonamidites, hydroxyls, carbonyls (e.g., carboxyl, ester and formyl groups), aldehydes, ketones, ethers, carbamoyl groups, thiols, sulfides, thiocarbonyls (e.g., thiolcarboxyl, thiolester and thiolformyl groups), thioethers, mercaptans, sulfonic acids, sulfoxides, sulfates, sulfonates, sulfones, sulfonamides, sulfamoyls and sulfinyls. Illustrative of suitable metal binding domains include those chemical moieties containing at least one Lewis basic nitrogen, sulfur, phosphorous or oxygen atom or a combination of such nitrogen, sulfur, phosphorous and oxygen atoms. The carbon atoms of such moiety may be part of an aliphatic, cycloaliphatic or aromatic moiety. In addition to the organic Lewis base functionality, such moieties may also contain other substituent atoms and/or groups, such as alkyl, aryl and halogen.

1) Chelating Groups as the Metal Binding Domains

The term “chelating group” is art-recognized and includes a molecule, often an organic one, and often a Lewis base, having two or more unshared electron pairs available for donation to a metal ion. It should be noted that a chelating group or moiety is a subgroup of a metal binding domain (MBD) or a Lewis base. The term chelating group may also be viewed as moiety with at least two Lewis bases capable of making at least two simultaneous coordinate bonds with a transition metal ion. For the purpose of the present invention, a chelating group or moiety is a group or moiety pendant to the backbone or terminally attached capable of forming at least two coordinate bonds with metal ions. To be identified as chelating group or moiety, for the purpose of this invention, the moiety must be able to maintain its ability to form at least two coordinate bonding independent of its attachment to the backbone. A chelated metal ion is a metal ion coordinated or coordinately bonded to at least two electron pairs of the chelating group or moiety. The terms, “bidentate chelating group”, “tridentate chelating group”, and “tetradentate chelating group” are art-recognized and refer to chelating groups having, respectively, two, three, and four electron pairs readily available for simultaneous donation to a metal ion coordinated by the chelating group. Usually, the electron pairs of a chelating group forms coordinate bonds with a single metal ion; however, in certain examples, a chelating agent may form coordinate bonds with more than one metal ion, with a variety of binding modes being possible. It may be the case that the metal bridge may comprise more than a single metal ion (i.e., multiple metal ions) with bridging ligands, provided that the chelating moiety of the backbone and MBD of the active agent are capable of being connected through the metal ions and bridging ligands. For the purpose of the present specification the “chelating group” is the same as “chelating moiety” and is a single pendant or terminal portion of the molecule containing two or more electron pairs that can be donated to metal ions. The chelating moiety of the backbone can maintain its chelating function even it is detached from the backbone while keeping the integrity of the backbone intact. A polylactic acid backbone without modification, a polyamino acid backbone without modifications and without two histidines occurring within a 6 amino acid span of the sequence, and polysaccharides without modification do not have naturally occurring chelating groups or chelating moieties for the purpose of this specification.

The chelating moiety of the present invention may include polycarboxylic acids containing nitrogen (such as iminodiacetic acid or IDA, nitrilodiacetic acid or NDA, nitrilotriacetic acid or NTA; EDTA; DTPA and the like) where at least one of carboxylic groups or the amino group may be utilized for covalent linking of the chelate or chelator to the backbone component of the carrier. The chelating moiety of the present invention also be amine (primary or secondary) containing chelator where the amine may be utilized for covalent linking to the backbone component of the carrier (such as for example N,N-Bis(carboxymethyl)-lysine; Iminodiacetic acid and the like). The addition of metal ions to chelator can result in formation of coordinate complexes (metal-chelates) either at room temperature or at elevated temperatures. These metal-chelate complexes can coordinately bind to the metal binding domain of a metallopeptidase such as lysostaphin: added in a purified state; in water; in a buffer; or in the presence of bulk protein or blood plasma proteins. The addition will result in formation of drug-delivery compositions containing coordinate complexes formed between the metal-chelate and a metallopeptidase or derivatives. The amino acid sequence of metallopeptidases of the invention may include one or more histidines or cysteines which increase the stability of the complex formed with the compositions of the invention.

Examples of metal binding domains which are chelating groups or act as chelating groups and can be chemically linked the backbone include:
1,4,7,10-tetraazacyclododecane-N,N′,N″,N′″-tetraacetic acid;
1,4,7,10-tetraaza-cyclododecane-N,N′,N″-triacetic acid;
1,4,7-tris(carboxymethyl)-10-(2′-hydroxypropyl)-1,4,7,10-tetraazocyclodecane;
1,4,7-triazacyclonane-N,N′,N″-triacetic acid;
1,4,8,11-tetraazacyclotetra-decane-N,N′,N″,N′″-tetraacetic acid;
1,2-diaminocyclohexane-N,N,N′,N′-tetraacetic acid;
bis(aminoethanethiol)carboxylic acid;
diethylenetriamine-pentaacetic acid (DTPA);
ethylenediamine-tetraacetic acid (EDTA);
ethyleneglycoltetraacetic acid (EGTA);
ethylene-bis(oxyethylene-nitrilo)tetraacetic acid;
ethylenedicysteine;
Imidodiacetic acid (IDA);
N-(hydroxyethyl)ethylenediaminetriacetic acid;
nitrilotriacetic acid (NTA);
nitrilodiacetic acid (NDA);
triethylenetetraamine-hexaacetic acid (TTHA);
bisphosphonates such as pamidronate, etidronate, alendronate, ibandronate, zoledronate, risendronate and derivates thereof; or
   a polypeptide having the formula: (AₓH_{y})ₚ, wherein A is any amino acid residue, H is histidine, x is an integer from 0-6; y is an integer from 1-6; and p is an integer from 2-6.

2) Non-Chelating Groups as the Metal Binding Domains

Coordinate bonding that does not fit the description of chelation as discussed above is also part of the compositions of the present invention. This is when a metal ion has a single coordination bond with a single moiety. Similarly, when a metal ion has a single coordination bond with a single moiety (first moiety) and there is a second coordination bond of the same metal with a second moiety further away (for example, at least 15 atoms apart) from the first moiety. Because the Lewis basic groups function as the coordination site or sites for the metal cation, in certain embodiments, it may be preferable that the deformability of the electron shells of the Lewis basic groups and the metal cations be approximately similar. Such a relationship often results in a more stable coordination bond. For instance, sulfur groups may be desirable as the Lewis basic groups when the metal cation is a heavy metal. Some examples include the oligopeptides such as glutathione and cysteine, mercaptoethanol amine, dithiothreitol, amines and peptides containing sulfur and the like. Nitrogen containing groups may be employed as the Lewis basic groups when smaller metal ions are the metal. Alternatively, for those applications in which a less stable coordination bond is desired, it may be desirable that the deformability be dissimilar.

C) Metal Ions

The present invention contemplates the use of a variety of different metal ions. The metal ion may be selected from those that have usually two, three, four, five, six, seven or more coordination sites. A non-limiting list of metal ions for which the present invention may be employed (including exemplary and non-limiting oxidation states for them) includes Co³⁺, Cr³⁺, Hg²⁺, Pd²⁺, Pt⁴⁺, Pd²⁺, Pt⁴⁺, Rh³⁺, Ir³⁺, Ru³⁺, Co²⁺, Ni²⁺, Cu²⁺, Zn²⁺, Cd²⁺, Cd²⁺, Pb²⁺, Mn²⁺, Fe³⁺, Fe²⁺, Tc, Au³⁺, Au⁺, Ag⁺, Cu⁺, MoO₂²⁺, Ti³⁺, Ti⁴⁺, CH₃Hg⁺, and Y⁺³. In another embodiment, the non-limiting list of metal ions for which the present invention may be employed includes Zn²⁺, Ni²⁺, Co²⁺, Fe²⁺, Mn²⁺, and Cu²⁺. The metal ion contained in the metal bridge between the carrier and the active agent metallopeptidase may have a therapeutic use itself, but it cannot serve as the active agent. In another embodiment of the invention the metal ion is a transition metal ion.

D) Protective Chains

Examples of protective chains (interchangeably referred to as protective side chains, hydrophilic protective chains) include poly(ethylene glycol), which may be esterified by dicarboxylic acid to form a poly(ethylene glycol) monoester; methoxy poly(ethylene glycol) monoester (MPEG) or a co-polymer of poly(ethylene glycol) and poly(propylene glycol) monoester preferably in a form of an ester with a dicarboxylic acid giving the terminal of this co-polymers a carboxyl group that can be used to covalently link it to a backbone (see above). Other forms include poly(ethylene glycol)-carboxyl; methoxy poly(ethylene glycol)-carboxyl; poly(ethylene glycol)-carboxymethyl; methoxy poly(ethylene glycol)-carboxymethyl; poly(ethylene glycol) monoamine; methoxy poly(ethylene glycol) monoamine; poly(ethylene glycol) hydrazide; methoxy poly(ethylene glycol) hydrazide; methoxy poly(ethylene glycol) imidazolide block-co-polymer of poly (ethylene glycol) and one or several polymers represented by polyaminoacid, polysaccharide, polyamidoamine, polyethyleneimine where these blocks are preferably alternated to give a preferably linear block-co-polymer. The overall molecular weight of a protective chain is preferentially larger than 300 Daltons but preferably not exceeding 10,000 Daltons. In one embodiment, one or more protective chains are linked to the polymeric or aliphatic backbone by a single linkage.

In one example provided herein, a composition of the present invention comprises a linear polymeric backbone with a degree of polymerization in the range of 2-10,000 to which independently and covalently linked are methoxypolyethylene glycol (mPEG) protective chains with a mass of 300-25,000 Daltons and chelating groups, where said protective chains and chelating groups are independently linked or pendant to the backbone. In another example, the degree of polymerization of the polymeric backbone is in the range of 25-1,000. In still another example, the degree polymerization of polymeric backbone is in the range of 50 to 300.

E) Active Agents: Metallopeptidases

Metallopeptidases, interchangeably referred to as metalloproteinases or metalloproteases are art-recognized as enzymes whose catalytic mechanism involves a metal or enzymes that have a metal in their active sites. Metallopeptidases include metalloexopeptidases and metalloendopeptidases. The carriers of the present invention can bind to all metallopeptidase, metalloexopeptidase, and metalloendopeptidase active agents and derivatives, fragments, and analogs thereof. Metalloendopeptidases, also known as a metalloproteinase endopeptidases, are art-recognized and form a group of endopeptidases that contain a metal in their structure and cleave a peptide bond within the peptide's internal structure. Metalloexopeptidases of the present invention, also known as metallocarboxypeptidases or C-terminal metallopeptidases, include enzymes that hydrolyze the carboxy-terminal (C-terminal) end of a peptide bond and whose catalytic mechanism involves a metal or whose active sites contain a metal. Metallopeptidases, metalloexopeptidases and metalloendopeptidases contain metal ions that can be shared with the chelating group of the carrier.

Metallopeptidases and their derivatives, fragments and analogs may be produced by recombinant techniques from DNA constructs that may or may not contain a preproeyzme or a proenzyme sequence. The metallopeptidase active agents of the present invention may or may not be recombinant products. If produced recombinantly it may or may not involve the use of specific promoter. The metallopeptidase active agents of the present invention may be product of recombinant production in mammalian cells that may or may not involved the DNA sequence modification that prevents glycosylation. The metallopeptidase active agents of the present invention may be native version purified from organism that naturally produces the metallopeptidase. The metallopeptidase active agents of the present invention may be purified from an organism. For example, lysostaphin, an exemplary metalloendopeptidase, can be purified from organism that naturally produces it, such as Staphylococcus simulans or Staphylococcus staphylolyticus.

1) Metalloendopeptidases

Exemplary metalloendopeptidases of the present invention are listed in but not limited to those in Table 1 and include all peptidases with the EC numbers (Enzyme Commission numbers as determined by the International Union of Biochemistry and Molecular Biology) designation EC 3.4.24. The Enzyme Commission number is an internationally-accepted numerical classification scheme for enzymes, based on the chemical reactions they catalyze. These and other metallopeptidases are discussed in further detail below. The carriers of the present invention can bind to all metalloendopeptidases as well as analogs, derivatives, and fragments thereof.

TABLE 1
Exemplary Metalloendopeptidases

| EC Number | Metalloendopeptidase |
|---|---|
| EC 3.4.24.1 | atrolysin A |
| EC 3.4.24.2 | Sepia proteinase |
| EC 3.4.24.3 | microbial collagenase |
| EC 3.4.24.4 | microbial metalloproteinases |
| EC 3.4.24.5 | lens neutral proteinase |
| EC 3.4.24.6 | leucolysin |
| EC 3.4.24.7 | interstitial collagenase |
| EC 3.4.24.8 | Achromobacter iophagus collagenase |
| EC 3.4.24.9 | Trichophyton schoenleinii collagenase |
| EC 3.4.24.10 | Trichophyton mentagrophytes |
| | keratinase |
| EC 3.4.24.11 | neprilysin |
| EC 3.4.24.12 | envelysin |
| EC 3.4.24.13 | IgA-specific metalloendopeptidase |
| EC 3.4.24.14 | procollagen N-endopeptidase |
| EC 3.4.24.15 | thimet oligopeptidase |
| EC 3.4.24.16 | neurolysin |
| EC 3.4.24.17 | stromelysin 1 |
| EC 3.4.24.18 | meprin A |
| EC 3.4.24.19 | procollagen C-endopeptidase |
| EC 3.4.24.20 | peptidyl-Lys metalloendopeptidase |
| EC 3.4.24.21 | astacin |
| EC 3.4.24.22 | stromelysin 2 |
| EC 3.4.24.23 | matrilysin |
| EC 3.4.24.24 | gelatinase a |
| EC 3.4.24.25 | vibriolysin |
| EC 3.4.24.26 | pseudolysin |
| EC 3.4.24.27 | thermolysin |
| EC 3.4.24.28 | bacillolysin |
| EC 3.4.24.29 | aureolysin |
| EC 3.4.24.30 | coccolysin |
| EC 3.4.24.31 | mycolysin |
| EC 3.4.24.32 | beta-Lytic metalloendopeptidase |
| EC 3.4.24.33 | peptidyl-Asp metalloendopeptidase |
| EC 3.4.24.34 | neutrophil collagenase |
| EC 3.4.24.35 | gelatinase B |
| EC 3.4.24.36 | leishmanolysin |
| EC 3.4.24.37 | saccharolysin |
| EC 3.4.24.38 | gametolysin |
| EC 3.4.24.39 | deuterolysin |
| EC 3.4.24.40 | serralysin |
| EC 3.4.24.41 | atrolysin B |
| EC 3.4.24.42 | atrolysin C |
| EC 3.4.24.43 | atroxase |
| EC 3.4.24.44 | atrolysin E |
| EC 3.4.24.45 | atrolysin F |
| EC 3.4.24.46 | adamalysin |
| EC 3.4.24.47 | horrilysin |
| EC 3.4.24.48 | ruberlysin |
| EC 3.4.24.49 | bothropasin |
| EC 3.4.24.50 | bothrolysin |
| EC 3.4.24.5 | 1 ophiolysin |
| EC 3.4.24.52 | trimerelysin I |
| EC 3.4.24.53 | trimerelysin II |
| EC 3.4.24.54 | mucrolysin |
| EC 3.4.24.55 | pitrilysin |
| EC 3.4.24.56 | insulysin |
| EC 3.4.24.57 | O-sialoglycoprotein endopeptidase |
| EC 3.4.24.58 | russellysin |
| EC 3.4.24.59 | mitochondrial intermediate peptidase |
| EC 3.4.24.60 | dactylysin |
| EC 3.4.24.61 | nardilysin |
| EC 3.4.24.62 | magnolysin |
| EC 3.4.24.63 | meprin B |
| EC 3.4.24.64 | mitochondrial processing peptidase |
| EC 3.4.24.65 | macrophage elastase |
| EC 3.4.24.66 | choriolysin L |
| EC 3.4.24.67 | choriolysin H |
| EC 3.4.24.68 | tentoxilysin |
| EC 3.4.24.69 | bontoxilysin |
| EC 3.4.24.70 | oligopeptidase A |
| EC 3.4.24.71 | endothelin-converting enzyme 1 |
| EC 3.4.24.72 | fibrolase |
| EC 3.4.24.73 | jararhagin |
| EC 3.4.24.74 | fragilysin |
| EC 3.4.24.75 | glycyl-glycine endopeptidase |
| | (lysostaphin) |
| EC 3.4.24.76 | flavastacin |
| EC 3.4.24.77 | snapalysin |
| EC 3.4.24.78 | gpr endopeptidase |
| EC 3.4.24.79 | pappalysin-1 |
| EC 3.4.24.80 | membrane-type matrix metalloproteinase-1 |
| EC 3.4.24.81 | ADAM10 endopeptidase |
| EC 3.4.24.82 | ADAMTS-4 endopeptidase |
| EC 3.4.24.83 | anthrax lethal factor endopeptidase |
| EC 3.4.24.84 | Ste24 endopeptidase |
| EC 3.4.24.85 | S2P endopeptidase |
| EC 3.4.24.86 | ADAM 17 endopeptidase |

Carriers of the present invention can bind metalloendopeptidases and analogs, derivatives, and fragments thereof. In specific embodiments carriers of the present invention bind gylcyl-glycyl metalloendopeptidases. Glycyl-glycyl metalloendopeptidases are art recognized, and are a group of metal containing enzymes capable of recognizing and cleaving a glycyl-glycyl amide bond. An example of this kind of enzyme, lysostaphin, is art-recognized and is bacteriolytic for Staphylococcus aureus. This includes derivatives and fragments of lysostaphin that have substantially the same biological effect as naturally occurring lysostaphin. The lysostaphin may be isolated or synthetically prepared. Derivatives and fragments may also be isolated or synthetically prepared. It is possible that certain derivatives of lysostaphin may have several metal binding domains which may or may not be chelating moietie(s). In one embodiment, a derivate of lysostaphin can be generated by truncation of the amino acid sequence or addition of other amino acids or functional groups such as a chelating group. In one embodiment lysostaphin (including its analogs, derivatives and fragments) comprises a metal binding domain capable of coordinate bonding with the metal ion, thus completing a bridge between lysostaphin and the chelating group covalently linked to the backbone of the carrier. Lysostaphin naturally contains at least one MBD, which may be used for binding to the carriers described above. Lysostaphin, therefore, supplies an MBD naturally such that there is no need to provide one synthetically. Lysostaphin may be loaded to the carrier of the present invention mixing a carrier solution with a lysostaphin solution at temperature between 15 to 37 degrees Celsius. The loaded carrier can be lyophilized and reconstituted prior to use. The lysostaphin of the present invention or metalloendopeptidases in general can be further modified to contain a chelating group to enhance binding to the carriers of the present invention. Chelating groups that can be used to modify lysostaphin includes all those listed in section above.

Insulysin, an active agent of the present invention, is an enzyme that catalyzes the degradation of insulin, glucagon and other polypeptides. It is inhibited by bacitracin, chelating agents EDTA and 1,10-phenanthroline, and by thiol-blocking reagents such as N-ethylmaleimide, but not phosphoramidon.

Lysostaphin, an active agent of the present invention, is a peptidase enzyme produced by certain strains of staphylococcus microorganisms with antibacterial activity against staphylococci. Lysostaphin is a 25-kDa peptidase produced by Staphylococcus simulans which cleaves a glycine-glycine bond unique to an inter-peptide cross-bridge of the staphylococcus aureus cell wall with EC number designation of EC 3.4.24.75. Lysostaphin is an exemplary metalloendopeptidase, more specifically a glycyl-glycyl metalloendopeptidase.

Pregnancy-Associated Plasma Protein-A, an active agent of the present invention, is a product of the placenta, and decidua, secreted into the maternal circulation during pregnancy. It has been identified as an IGF binding protein (IGFBP)-4 protease that proteolyzes IGFBP-4 and thus increases IGF bioavailability. It is found also in human fibroblast, ovarian follicular fluid, and granulose cells. The enzyme is a heterotetramer of about 500-kDa.

Procollagen N-Endopeptidase, an active agent of the present invention, is an extracellular endopeptidase which excises a block of peptides at the amino teiluinal, nonhelical region of the procollagen molecule with the formation of collagen. It has EC number designation of EC 3.4.24.14. Absence or deficiency of the enzyme causes accumulation of procollagen which results in the inherited connective tissue disorder-dermatosparaxis.

Pronase, an active agent of the present invention, is a proteolytic enzyme obtained from Streptomyces griseus.

Thermolysin, an active agent of the present invention, is a thermostable extracellular metalloendopeptidase containing four calcium ions. It has EC number designation of EC 3.4.24.27.

Neprilysin, an active agent of the present invention, is a metallomembrane endopeptidase enzyme, a major constituent of kidney brush-border membranes. In one embodiment, use of neprilysin as an active agent of the present invention is useful for the treatment of Alzheimer's disease and related dementias. It is naturally found in the brain and is interchangeably known as the common acute lymphoblastic leukemia antigen (CALLA). It has EC number designation of EC 3.4.24.11.

Collagenase, an active agent of the present invention, is a proteolytic enzyme that acts on one or more of the collagens.

Gelatinase, an active agent of the present invention, such as Pepsin B is a metalloproteinase that hydrolyzes gelatin and a number of types of collagen. Pepsin Gelatinase is a class of enzymes that catalyzes the degradation of gelatin by acting on the peptide bonds.

Matrix metalloproteinase, an active agent of the present invention, is an endopeptidase subfamily that hydrolyzes extracellular proteins, especially collagens and elastin. By regulating the integrity and composition of the extracellular matrix, these enzymes play a role in the control of signals elicited by matrix molecules that regulate cell proliferation, differentiation, and death. Matrix metalloproteinase is a family of zinc-dependent metalloendopeptidases that are involved in the degradation of extracellular matrix component.

PHEX (Phosphate Regulating Neutral Endopeptidase), an active agent of the present invention, is a membrane-bound metalloendopeptidase that may play a role in the degradation or activation of a variety of peptide hormones and intracellular signaling peptide and proteins. Genetic mutations that result in loss of function of this protein are a cause of hypophosphosphatemic rickets, x-linked dominant.

ADAM Proteins are a family of membrane-anchored glycoproteins, active agents of the present invention, and contain a disintegrin and a metalloprotease domain. They are responsible for the proteolytic cleavage of many transmembrane proteins and the release of their extracellular domain.

2) Metalloexopeptidases

Exemplary metalloexopeptidases, interchangeably referred to as metallocarboxypeptidases, of the present invention are listed in but not limited to those in Table 2 and include all peptidases with the EC number designation EC 3.4.17. The carriers of the present invention can bind to all metalloexopeptidases and analogs, derivatives, and fragments thereof.

TABLE 2
Exemplary Metallocarboxypeptidases

| EC Number | Metallocarboxypeptidase |
|---|---|
| EC 3.4.17.1 | carboxypeptidase A |
| EC 3.4.17.2 | carboxypeptidase B |
| EC 3.4.17.3 | lysine carboxypeptidase |
| EC 3.4.17.4 | Gly-X carboxypeptidase |
| EC 3.4.17.5 | aspartate carboxypeptidase |
| EC 3.4.17.6 | alanine carboxypeptidase |
| EC 3.4.17.7 | acylmuramoyl-alanine |
| | carboxypeptidase |
| EC 3.4.17.8 | muramoylpentapeptide |
| | carboxypeptidase |
| EC 3.4.17.9 | carboxypeptidase S |
| EC 3.4.17.10 | carboxypeptidase E |
| EC 3.4.17.11 | glutamate carboxypeptidase |
| EC 3.4.17.12 | carboxypeptidase M |
| EC 3.4.17.13 | Muramoyltetrapeptide carboxypeptidase |
| EC 3.4.17.14 | Zinc D-Ala-D-Ala carboxypeptidase |
| EC 3.4.17.15 | carboxypeptidase A2 |
| EC 3.4.17.16 | Membrane Pro-X carboxypeptidase |
| EC 3.4.17.17 | tubulinyl-Tyr carboxypeptidase |
| EC 3.4.17.18 | carboxypeptidase T |
| EC 3.4.17.19 | Carboxypeptidase Taq |
| EC 3.4.17.20 | Carboxypeptidase u |
| EC 3.4.17.21 | Glutamate carboxypeptidase II |
| EC 3.4.17.22 | Metallocarboxypeptidase D |

### Micelle, Reverse Micelle, Colloid, Liposome, Emulsion, or Hydrogel Supramolecular Structures

The compositions of the present invention can form supramolecular structures selected from but not limited to a micelle, reverse micelle, colloid, liposome, emulsion, and hydrogel.

The composition of the present invention, comprising an aliphatic chain with covalently linked chelating groups, is amphipathic (containing both hydrophobic and hydrophilic domains). Furthermore, the composition of the present invention comprising an aliphatic chain with covalently linked chelating groups and covalently linked protective chains is also amphipathic. In addition the composition of the present invention comprising a hydrophobic polyaminoacid as the polymeric backbone with covalently linked chelating groups is also amphipathic. The composition of the present invention comprising a hydrophobic polyaminoacid as the polymeric backbone with covalently linked chelating groups and covalently linked protective chains is also amphipathic. These compositions comprising an aliphatic backbone or a hydrophobic polyamino acid backbone can organize and be part of vesicular structures such as liposomes, micellar, or reverse micellar structures. In the presence of a metal chelated to the chelating group and a metallopeptidase active agent with a metal binding domain coordinately bonded to the metal ion, the metallopeptidase active agent can organize and associate with the vesicular structures. Liposomes can contain an aqueous volume that is entirely enclosed by a membrane composed of lipid molecules (usually phospholipids). Micelles and reverse micelles are microscopic vesicles that contain amphipathic molecules but usually do not contain an aqueous volume that is entirely enclosed by a membrane. In micelles the hydrophilic part of the amphipathic compound is on the outside (on the surface of the vesicle) whereas in reverse micelles the hydrophobic part of the amphipathic compound is on the outside. The reverse micelles contain a polar core that can dissolve both water and macromolecules within the reverse micelle. As the volume of the core aqueous pool increases the aqueous environment begins to match the physical and chemical characteristics of bulk water. The resulting reverse micelle can be referred to as a microemulsion of water in oil. It is the object of the present invention to disclose a composition comprising an aliphatic or hydrophobic backbone, a chelating moiety covalently linked to the aliphatic or hydrophobic backbone, a metal ion chelated to the chelating moiety, a metallopeptidase active agent (such as lysostaphin) with metal binding domain coordinately bonded to the metal ion, and optionally a protective chain covalently linked to the backbone; wherein the composition is a component of any one of micelle, reverse micelle, colloid, liposome, emulsion, or hydrogel.

In water, when sufficient concentrations of the two or more components that make up a micelle are present, the components can spontaneously aggregate into thermodynamically stable micelles. The micelle particles can assume a micro-spheroidal shape and possess, in essence, a double layer. The core “layer” forms because of the hydrophobic interactions between, for example, aliphatic chains. Similarly, the surface “layer” forms because of the corresponding hydrophilic interactions of, for example, a hydrophilic metal ion and active agent with water. A net charge usually will exist around the surface of the micelle, since the hydrophilic segment is the metal ion and the active agent. If a hydrophilic protective chain is covalently linked to the aliphatic chain, the hydrophilic chain will be on the outside of the micelle or liposome and the active agent coordinately bonded to the chelated metal ion will be on the inside of the micelle or liposome.

### Sustained Release

Coordinated metallopeptidase active agents according to the present invention preferably results in longer circulation in the body, more stability in the blood, and can be more conveniently administered (for example, quicker administrations such as through bolus instead of infusion, and less frequent administrations, e.g. once every few days instead of infusion or once a day). Often chronic administration of a metallopeptidase active agent may be immunogenic. Carrier based formulations generally result in less immunogenicity than PEG based delivery systems so the metallopeptidase is expected to be less immunogenic in compositions of the present inventions. “Direct PEGylation” of the active agent is the direct bonding of the metallopeptidase to PEG and can results in loss of activity. A metallopeptidase coordinated with the chelated metal which is covalently linked to the backbone of the carrier with protective side chains, preferably, can result in a stable, long circulating alternative to PEGylation. The carriers of the present invention may act as a cryoprotectant and macromolecular stabilizer preserving metallopeptidase active agent in solution as well as during the lyophilization and reconstitution process.

When the carrier of the present invention is formulated with a metallopeptidase active agent, a release of the active agent for an extended period will be observed as evident from the sustained presence of the active agent in the blood compared to administering the active agent alone. The association of carrier with the active agent is defined by specific dissociation constant (Kd) that can easily be determined by those skilled in the art. The release is determined by the concentration of free active agent such that the when the free active agent concentration goes down (due to degradation or elimination by the body) and no longer satisfies the Kd, more active agent will be release to satisfy the Kd. The Kd is the product of concentration of free active agent and the concentration of chelated metal ions (not coordinately bonded to the active agent) divided by the concentration of the active agent coordinately bonded to the chelated metal ion. For the compositions of the present invention that form supramolecular structures such as micelles, liposomes and other structures, the release rate preferably follows the Kd but due to compartmentalization the Kd is satisfied in each specific compartment. However, long term mixing of the various compartments can result in eventual release of the active agent into the surrounding environment. In both cases whether compartmentalization is involved or not, a release profile results in prolonged delivery (over, for example 1 to about 4,000 hours, or alternatively about 4 to about 1500 hours) of effective amounts (e.g., about 0.00001 mg/kg/hour to about 10 mg/kg/hour) of the active agent. The advantage of the formulation is less frequent bolus administration from continuous to once a day or even once a week. This provides a more constant level of active agent in the blood with less fluctuation compared to an unformulated active agent. The frequency of bolus administration varies according to the needs of the patient and can be determined by those skilled in the art.

### Therapeutic Uses

A “patient,” “subject” or “host” to be treated with the composition of the present invention may mean either a human or non-human animal, preferably human. The metalloendopeptidases and the metalloexopeptidases of the present invention are useful in the treatment of such diseases and disorders such as but not limited to bacterial infections, cancer and related neoplastic diseases, and Alzheimer's disease. In one embodiment, the compositions of the present invention may be used in the manufacture of a medicament for any number of uses, including for example treating any disease or other treatable condition of a patient.

A “therapeutic effect,” as that term is used herein, encompasses a therapeutic benefit and/or a prophylactic benefit. By therapeutic benefit is meant eradication or amelioration of the underlying disorder being treated. Also, a therapeutic benefit is achieved with the eradication or amelioration of one or more of the physiological symptoms associated with the underlying disorder such that an improvement is observed in the patient, notwithstanding that the patient may still be afflicted with the underlying disorder. For prophylactic benefit, the compositions may be administered to a patient at risk of developing a particular disease, or to a patient reporting one or more of the physiological symptoms of a disease, even though a diagnosis of this disease may not have been made. A prophylactic effect includes delaying or eliminating the appearance of a disease or condition, delaying or eliminating the onset of symptoms of a disease or condition, slowing, halting, or reversing the progression of a disease or condition, or any combination thereof.

A) Bacterial Infections

In one embodiment, the metallopeptidases of the present invention are useful in the treatment of bacterial infections. Exemplary active agents for treatment include lysostaphin, a glycyl-glycyl metalloendopeptidase.

Lysostaphin, an exemplary metallopeptidase, is a glycyl-glycyl metalloendopeptidase. Lysostaphin cleaves pentaglycine cross-bridges in the cell wall peptidoglycan of gram positive bacteria. S. aureus is particularly susceptible to the bacteriolytic effects of this enzyme since its cell wall contains a high proportion of pentaglycine cross-bridges. Lysostaphin is a potential systemic therapy for treating multidrug-resistant S. aureus mediated infections including endocarditis, osteomyelitis, catheter related infections, and MRSA-mediated community acquired furunculosis and pneumonia.

However, to date, lysostaphin has been developed only as a topical treatment for S. aureus due to the following limitations. Lysostaphin has a short half life in vivo with >90% reduction in serum levels occurring in less than one hour. This may be due to a combination of renal ultrafiltration of this protein, degradation by proteases and/or its clearance by reticuloendothelial system. Lysostaphin is immunogenic and repeated doses have demonstrated decreasing efficacy due to the development of neutralizing antibodies in the host. The development of resistance to lysostaphin has been reported in vitro and in vivo with low concentrations/doses of lysostaphin in oxacillin-resistant strains of S. aureus. Therefore a longer-circulating and targeted formulation of lysostaphin would enable the ability to obtain a high blood and infection site concentration and, as a result, minimize or avoid the development of resistance. In part, the present invention is directed towards a novel lysostaphin delivery system that overcomes the above limitations, and methods of making and using the same.

Infection with Staphylococcus aureus, a leading cause of nosocomial infection, doubles the cost, length of stay and death rate of a typical hospitalized patient. Importantly, infection with an isolate of methicillin resistant Staphylococcus aureus (MRSA), the first line of treatment, more than doubles the death rate. This is because available therapies for hospital-acquired MRSA, which is typically multiresistant, are limited. The emergence of high-level vancomycin resistance in this pathogen predicts a worsening situation if new treatments are not developed. Existing therapies for resistant S. aureus include vancomycin and the more recently approved linezolid (for pneumonia) and daptomycin (approved so far for skin and soft tissue infections). Additionally, there are additional antibiotics in late stages of development active against MRSA including the two glycopeptides (oritavancin and dalbavancin) and the minocycline derivative tigecycline. However, given the changing epidemiology of S. aureus infection and the fact that the indications for these agents is as yet unknown, an active agent such as lysostaphin offers a promising alternative for the treatment of S. aureus infections. Its activity against both actively growing and sessile bacteria offers the potential for therapeutic efficacy against catheter/device related infections and superior activity against endocarditis. Bactericidal therapies for S. aureus are particularly needed for the infections as detailed below.

1) Primary Bloodstream Infections

Primary bloodstream infection (BSI) is a leading, infectious complication among critically ill patients. It affects approximately 1% of all hospitalized patients, with an incidence rate of 5 per 1,000 central-line days and represents about 15% of all nosocomial infections. BSI increases the mortality rate, prolongs patient stay in an intensive care unit (ICU) and in the hospital, and generates substantial extra costs.

2) Bacteremia in Neutropenic Patients

Bacteremia in neutropenic patients immunocompromised due to immunosuppression, chemotherapy or a disease state such as AIDS or diabetes is frequently caused by Staphylococcus aureus (MRSA). Due to the necessity to completely eradicate an infection in immunocompromised individuals, bactericidal antibiotics are recommended for therapy. It is feared that effective treatment options for this increasing population of individuals will become progressively limited due to the rapid emergence and dissemination of antimicrobial resistance in nosocomial pathogens.

3) Bacterial Infective Endocarditis (IE)

IE is a serious and life-threatening infection of the heart valves. The current incidence is 4-6 cases per 100,000 of population per year. Despite modem antibiotic and surgical therapies, IE retains an overall mortality of 15-40%. S. aureus is a common cause of IE, and carries the highest mortality among IE pathogens. Bacterial vegetations in infectious endocarditis (1E) protect the invading organism from host defenses making it necessary to administer a bactericidal rather than a bacteriostatic antibiotic to obtain a cure. Recommended therapy includes the glycopeptides teicoplanin or vancomycin; (3-lactams including oxacillin and methicillin; aminoglycosides; rifampin or quinolones. Additionally, combinations of agents that demonstrate bactericidal activity against the etiological agent have been successfully used to obtain a cure. However, the increasing resistance of the etiological agents of IE to these antibiotics is drastically limiting treatment options and there is serious concern that resistance may develop to all available antibiotics. Considering that the mortality rate for IE prior to the antibiotic era was 100%, this is indeed a daunting prospect.

4) Osteomyelitis

Osteomyelitis is another situation where use of a bactericidal agent is recommended. This condition is usually diagnosed when stationary growths of bacteria have established in the bone complicating therapy. When chronic, this disease is notoriously resistant to antibiotics. The ultimate goal of osteomyelitis treatment is to eradicate infection and prevent recurrence using antibiotic therapy which typically extends for a number of weeks. The most common cause of osteomyelitis is Staphylococcus, and, as is the case with endocarditis, the emerging resistance of this pathogen to a number of antibiotics is limiting therapeutic options.

5) Infections Involving Bacterial Biofilms

Infections involving bacterial biofilms, which are complex communities of bacterial cells that can form on the surfaces of prosthetic implants and catheters, are difficult to treat with antibiotics. It is estimated that 3 to 5% of all central venous catheters (CVCs) become infected with a biofilm and S. aureus appears to be the most common etiological agent. Although existing antibiotics are effective against bacteria in the planktonic state, none are effective against the same bacteria in a biofilm. For infected CVCs, which have an attributable mortality rate of 10-20%, the reported efficacy of systemic antibiotic therapy alone is only 25-32%. Thus this type of infection usually necessitates removal of the infected device. Since there is no effective treatment current efforts are focused on prevention of infection.

6) Community-acquired MRSA Infections

The emergence and increasing incidence of severe community-acquired MRSA infections (skin/soft tissue and pneumonia) in patients with no known risk factors have serious public health implications. Recent reports indicate that CA-MRSA currently accounts for 60% of community-acquired S. aureus infections whereas ten years ago it only accounted for 10%, illustrating the increasing incidence of this pathogen. Community-acquired MRSA is distinguishable from nosocomial-acquired MRSA based on its genotype, methicillin resistant cassette element and antibiotic susceptibility profile. CA-MRSA is more likely to carry Panton-Valentine Leukocidin (PLV) virulence factor associated with severe necrotizing pneumonia and skin/soft tissue infections. Therapeutic options for these infections are untested and the potential exists for high morbidity and mortality. Indeed, despite the susceptibility of this pathogen to non-beta lactams, severe infections with this pathogen can carry a high rate of mortality: a recent study of adolescents with severe community acquired MRSA infections reported a mortality rate of 20%. In one embodiment carrier of the present invention delivering lysostaphin will serve as a therapeutic option for these types of infection, particularly considering that its bactericidal activity may eradicate the infection and preventing recurrence.

B) Alzheimer's Disease

In another embodiment, the metallopeptidases of the present invention are useful in the treatment of Alzheimer's diseases. Exemplary active agents for treatment include neprilysin, a metalloendopeptidase. Neprilysin, an active agent of the present invention, is a metallomembrane endopeptidase enzyme, a major constituent of kidney brush-border membranes. It is also found in the brain and is identical to common acute lymphoblastic leukemic antigen. It has EC number designation of EC 3.4.24.11.

### Administration and Dosages

A “patient,” “subject” or “host” to be treated with the composition of the present invention may mean either a human or non-human animal.

The term “pharmaceutically acceptable excipient” is art-recognized and refers to a pharmaceutically-acceptable material, composition or vehicle, such as a liquid or solid filler, diluent, solvent or encapsulating material, involved in carrying or transporting any supplement or composition, or component thereof, from one organ, or portion of the body, to another organ, or portion of the body. Each excipient is “acceptable” in the sense of being compatible with the other ingredients of the supplement and not injurious to the patient. Some examples of materials which may serve as pharmaceutically acceptable excipients include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations.

The dosage of the metallopeptidase active agent of the present invention will vary depending on the symptoms, age and body weight of the patient, the nature and severity of the disease or disorder, the route of administration, and other drugs/active agents being administered to the patient in conjunction. In embodiments where the active agent is lysostaphin, the dosage will depend on the severity of the infections, and the form of other supplemental antibiotics. Any of the subject formulations may be administered in a single dose or in divided doses. Dosages for the metallopeptidase formulation of the present invention may be readily determined by techniques known to those of skill in the art or as taught herein. Also, the present invention contemplates mixtures of one or more of the formulations of the present invention along with one or more antibiotics or other therapeutic agents. In particular embodiments, the carrier containing metallopeptidases of the present invention may be administered along with any one or more of other antibiotics selected from: Amoxicillin, Ampicillin, Azidocillin, Azlocillin, Aztreonam, Bacitacin, Benzathine benzylpenicillin, Benzathine phenoxymethylpenicillin, Benzylpenicillin(G), Biapenem, Carbenicillin, Cefacetrile, Cefadroxil, Cefalexin, Cefaloglycin, Cefalonium, Cefaloridine, Cefalotin, Cefapirin, Cefatrizine, Cefazedone, Cefazaflur, Cefazolin, Cefradine, Cefroxadine, Ceftezole, Cefaclor, Cefamandole, Cefminox, Cefonicid, Ceforanide, Cefotiam, Cefprozil, Cefbuperazone, Cefuroxime, Cefuzonam, cephamycin (such as Cefoxitin, Cefotetan, Cefinetazole), carbacephem (such as Loracarbef), Cefcapene, Cefdaloxime, Cefdinir, Cefditoren, Cefetamet, Cefixime, Cefinenoxime, Cefodizime, Cefoperazone, Cefotaxime, Cefpimizole, Cefpiramide, Cefpodoxime, Cefsulodin, Ceftazidime, Cefteram, Ceftibuten, Ceftiolene, Ceftizoxime, Ceftriaxone, oxacephem (such as Flomoxef, Latamoxef), Cefepime, Cefozopran, Cefpirome, Cefquinome, Ceftobiprole, Chloroamphenicol, Chlorohexidine, Clindamycin, Clometocillin, Cloxacillin, Colistin, Cycloserine, Daptomycin, Doripenem, Doxycycline, Epicillin, Ertapenem, Erythromycin, Faropenem, Fostomycin, Gentamycin, Imipenem, Linezolid, Mecillinam, Meropenem, Methicillin, Meticillin, Mezlocillin, Minocycline, Mupirocin, Nafcillin, Neomycin, Oxacillin, Panipenem, Penamecillin, Pheneticillin, Phenoxymethylpenicillin (V), Piperacillin, Polymyxin, Polymyxin B, Procaine benzylpenicillin, Propicillin, Quinupristin/dalfopristin, Ramoplanin, Rifampicin, Rifampin, Sulbenicillin, Teicoplanin, Tigecycline, Tigemonam, Trimethoprim/sulfamethoxazole, and Vancomycin. In particular embodiments, the carrier containing metallopeptidases of the present invention may be administered along with any one or more of other antibiotics selected from: Aztreonam, Bacitacin, Ceftazidime, Chloroamphenicol, Chlorohexidine, Clindamycin, Daptomycin, Doxycycline, Erythromycin, Gentamycin, Linezolid, Methhicillin, Minocycline, Mupirocin, Neomycin, Oxacillin, Polymyxin, Quinupristin/dalfopristin, Rifampicin, Rifampin, Teicoplanin, Temocillin, Ticarcillin, Tigecycline, Trimethoprim/sulfamethoxazole, and Vancomycin. In particular embodiments, the carrier containing metallopeptidases of the present invention may be administered along with any glycopeptide antibiotic in weight ratios of metallopeptidase to glycopeptide antibiotic ranging from 0.1:1 to 20:1. A more preferable range of weight ratios is from 0.5:1 to 7:1. The glycopeptides antibiotic may be selected from the group consisting of vancomycin, teicoplanin and ramoplanin. The composition of the present invention may be in a form suitable for intravenous, intramuscular, subcutaneous, intraperitoneal, intrathecal or topical administration.

The present invention also pertains to a method of treating a staphylococcal infection in a human subject comprising: administering composition of the present invention comprising a carrier with a metal bridge and lysostaphin; wherein lysostaphin is administered in an amount of from 1 mg to 150 mg/kg body weight/day to the human subject; and administering a beta-lactam antibiotic in an amount of from 50 to 250 mg/kg body weight/day to the human subject. The beta-lactam antibiotic may be administered along with the carrier and lysostaphin; such that the dose of beta-lactam in the human subject is antibiotic is from 100 to 200 mg/kg body weight/day. The beta-lactam antibiotic may be a penicillin, a cephalosporin, penem, a carbapenem, or a monobactam. The beta-lactam antibiotics that belong to penicillins include: aminopenicillins (such as Amoxicillin Ampicillin Epicillin); carboxypenicillins (such as Carbenicillin, Ticarcillin, Temocillin); ureidopenicillins (Azlocillin, Piperacillin, Mezlocillin); and others:(such as Mecillinam, SulbenicillinBenzylpenicillin (G), Azidocillin, Penamecillin, Clometocillin, Benzathine benzylpenicillin, Procaine benzylpenicillin, Phenoxymethylpenicillin (V), Propicillin, Benzathine, phenoxymethylpenicillin, Pheneticillin, Oxacillin, Cloxacillin, Meticillin, Nafcillin). The beta-lactam antibiotics that belong to cephalosporins are: Cefacetrile, Cefadroxil, Cefalexin, Cefaloglycin, Cefalonium, Cefaloridine, Cefalotin, Cefapirin, Cefatrizine, Cefazedone, Cefazaflur, Cefazolin, Cefradine, Cefroxadine, Ceftezole, Cefaclor, Cefamandole, Cefminox, Cefonicid, Ceforanide, Cefotiam, Cefprozil, Cefbuperazone, Cefuroxime, Cefuzonam, cephamycin, carbacephem, Cefcapene, Cefdaloxime, Cefdinir, Cefditoren, Cefetamet, Cefixime, Cefinenoxime, Cefodizime, Cefoperazone, Cefotaxime, Cefpimizole, Cefpiramide, Cefpodoxime, Cefsulodin, Ceftazidime, Cefteram, Ceftibuten, Ceftiolene, Ceftizoxime, Ceftriaxone, oxacephem, Cefepime, Cefozopran, Cefpirome, Cefquinome, and Ceftobiprole. The beta-lactam antibiotics that belong to carbopenems are: Biapenem, Doripenem, Ertapenem, Imipenem, Meropenem, and Panipenem. The beta-lactam antibiotic that is penem is Faropenem.

In certain embodiments, the dosage of a metallopeptidase formulation will generally be in the range of about 0.01 ng to about 1000 mg of metallopeptidase per kg body weight, specifically in the range of about 1 ng to about 100 mg of metallopeptidase per kg, and more specifically in the range of about 100 ng to about 20 mg of metallopeptidase per kg. The more preferable dose range is about 100 ng to about 20 mg of metallopeptidase per kg. The amount of metallopeptidase relative to the weight of the carrier in a formulation may be in the range of about 1% to 1000% of the weight of the carrier. More preferably the amount of metallopeptidase relative to the weight of the carrier in a formulation may be in the range of about 5% to 500% of the weight of the carrier. Even more preferably the amount of metallopeptidase relative to the weight of the carrier in a formulation may be in the range of about 10% to 100% of the weight of the carrier.

An effective dose or amount, and any possible affects on the timing of administration of the formulation, may need to be identified in the present invention. This may be accomplished by routine experiment as described herein, using one or more groups of animals (preferably at least 5 animals per group), or in human trials if appropriate. The effectiveness of the metallopeptidase formulation may be assessed by administering and assessing the effect of the administration by measuring one or more indices associated with the disease/disorder/infection of interest, and comparing the post-treatment values of these indices to the values of the same indices prior to treatment.

The precise time of administration and amount of any particular compound that will yield the most effective treatment in a given patient will depend upon the activity, pharmacokinetics, and bioavailability of the metallopeptidase, physiological condition of the patient (including age, sex, disease type and stage, general physical condition, responsiveness to a given dosage and type of medication), route of administration, and the like. The guidelines presented herein may be used to optimize the treatment, e.g., determining the optimum time and/or amount of administration, which will require no more than routine experimentation consisting of monitoring the subject and adjusting the dosage and/or timing.

Treatment may be initiated with smaller dosages which are less than the optimum dose of the compound. Thereafter, the dosage may be increased by small increments until the optimum therapeutic effect is attained.

The combined use of the metallopeptidase formulation of the present invention with other antibiotics or other therapeutic agents may reduce the required dosage for the metallopeptidase formulation. This is because the effect of other antibiotics or other therapeutic agents may be complimentary to the effect of the metallopeptidase formulation. In such combined therapy, the different active agents may be delivered together or separately, and simultaneously or at different times within the day.

Toxicity and therapeutic efficacy of the metallopeptidase formulation of the present invention may be determined by standard pharmaceutical procedures in cell cultures or experimental animals, e.g., for determining the LD₅₀, ED₅₀, MIC (Minimum concentration of the product that will still inhibit the growth of a test microorganism), and/or MBC (Minimum concentration of the product that will kill a- or bactericidal to a-test organism). Foiinulations that exhibit large therapeutic indices are preferred. Although foiinulations that exhibit toxic side effects may be used, care should be taken that the carrier—the metallopeptidase complex preferably accumulates at the desired site in order to reduce side effects.

The data obtained from the cell culture assays and animal studies may be used in formulating a range of dosage for use in humans. The dosage of any metallopeptidase formulations must provide a range of circulating concentrations in the blood that is above MIC with little or no toxicity. The dosage may vary within this range depending upon the dosage form employed and the route of administration utilized. For agents of the present invention, the therapeutically effective dose may be estimated initially from bacterial culture assays to obtain the MIC and the MBC. A dose of the formulation may be derived from animal models based on the dose that gives a circulating plasma concentration range above MIC and/or MBC as determined in cell culture. Such information may be used to more accurately determine useful doses in humans.

The carrier with metallopeptidases of the present invention may be used for external administration in a form of ointment, paste, cream or gels and may further contain excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

The carrier with metallopeptidases of the present invention may be used for external administration in a form of powder or spray and may further contain excipients such as lactose, talc, silicic acid, aluminum hydroxide, calcium silicates and polyamide powder, or mixtures of these substances. Sprays may additionally contain customary propellants, such as chlorofluorohydrocarbons and volatile unsubstituted hydrocarbons, such as butane and propane.

The carrier with metallopeptidases of the present invention may be used for external administration in a form of aerosol. This is accomplished by preparing an aqueous aerosol, liposomal preparation or solid particles containing the composition of the present invention but not covalently bonded to the solid. A non-aqueous (e.g., fluorocarbon propellant) suspension could be used. Sonic nebulizers may be used because they minimize exposing the agent to shear, which may result in degradation of the compound. Ordinarily, an aqueous aerosol is made by formulating an aqueous solution or suspension of the formulation together with conventional pharmaceutically acceptable carriers and stabilizers. The excipients and stabilizers vary with the requirements of the particular compound, but typically include non-ionic surfactants (Tweens, Pluronics, or polyethylene glycol), innocuous proteins like serum albumin, sorbitan esters, oleic acid, lecithin, amino acids such as glycine, buffers, salts, sugars or sugar alcohols. Aerosols generally are prepared from isotonic solutions.

Pharmaceutical compositions of this invention suitable for parenteral administration comprise one or more components of a supplement in combination with one or more pharmaceutically-acceptable sterile isotonic aqueous or non-aqueous solutions, dispersions, suspensions or emulsions, or sterile powders which may be reconstituted into sterile injectable solutions or dispersions just prior to use, which may contain antioxidants, buffers, bacteriostats, solutes which render the formulation isotonic with the blood of the intended recipient or suspending or thickening agents.

Examples of suitable aqueous and non-aqueous excipients which may be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity may be maintained, for example, by the use of coating materials, such as lecithin, by the maintenance of the required particle size in the case of dispersions, and by the use of surfactants.

### Kits

This invention also provides kits for conveniently and effectively implementing the methods of this invention. Such kits comprise any of the compounds of the present invention or a combination thereof, and a means for facilitating compliance with methods of this invention. Such kits, in the case of metallopeptidase formulations, provide a convenient and effective means for assuring that the subject to be treated takes the appropriate active in the correct dosage in the correct manner. The compliance means of such kits includes any means which facilitates administering the actives according to a method of this invention. Such compliance means include instructions, packaging, and dispensing means, and combinations thereof. Kit components may be packaged for either manual or partially or wholly automated practice of the foregoing methods. In other embodiments involving kits, this invention contemplates a kit including compositions of the present invention, and optionally instructions for their use. The sample can be liquids from many sources including serum, plasma, whole blood, urine, tissue extract, bacterial extracts, viral extracts, fungal extracts, or any samples in which the presence of metallopeptidases (for example lysostaphin) is suspected or needed to be quantified.

In one aspect, the present invention relates to a kit comprising a composition comprising: (i) a polymeric or aliphatic backbone (ii) a chelating moiety covalently linked or bonded to the backbone; (iii) a metal ion chelated to the chelating moiety by at least two coordinate bonds; (iv) a metallopeptidase active agent with a metal binding domain (MBD) (which may or may not be a chelator) coordinately bonded to the metal ion; and optionally (v) a protective chain covalently linked or bonded to the backbone. Uses for such kits include, for example, therapeutic applications. Such kits may have a variety of other uses, including, for example, imaging, targeting, diagnosis, therapy, vaccination, and the like.

### REFERENCES

The following patents and patent publications are incorporated by reference in their entirety. U.S. Pat. Nos. 7,452,533; 7,122,514; 7,091,332; 7,078,377; 6,395,299; 6,897,041; 6,875,903; 6,794,350; 6,776,824; 6,681,765; 6,620,585; 6,569,830; 6,566,062; 6,365,156; 6,315,996; 6,248,324; 6,056,955; 6,043,219; 6,028,051; 5,985,593; 5,961,975; 5,871,710; 5,866,140; 5,858,962; 5,763,585; 5,760,026; 5,703,040; 5,702,895; 5,270,176; 5,663,387; 5,605,672; 5,593,658; 4,980,163; 4,931,390; 4,810,567; 4,734,362; 4,513,083; 4,496,363 U.S. Published Patent Application No. 20080311216; 20080267943; 20080193912; 20080171804; 20080131457; 20080107707; 20080095756; 20080057049; 20070292404; 20070212340; 20070202051; 20070181133; 20070149694; 20070141145; 20060246055; 20060239960; 20060234219; 20060223071; 20060223070; 20060153857; 20060024365; 20060018934; 20060018933; 20050202476; 20050153370; 20050118198; 20050118159; 20050014932; 20040259162; 20040248199; 20040247605; 20040192581; 20040076624; US2003224974; 20030224000; 20030215436; 20030215433; 20030211995; 20030199432; 20030131439; 20030111075; 20030109017; 20020197637; 20020194629; 20020187136; 20020178509; 20020127587; 20020086020; 20020042078; 20020012982; 20020006406.

### EXAMPLES

The invention is further illustrated by the following Examples. The Examples are provided for illustrative purposes only, and are not to be construed as limiting the scope or content of the invention in any way.

Unless otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in this specification and attached claims are approximations that may vary depending upon the desired properties sought to be obtained by the present invention.

### Example 1

### Synthesis of PLPEG (lot#20020101)

Poly-L-lysine, hydrobromide (Sigma, Mw=48000, d.p. 200), 1 g was dissolved in 175 ml of 0.1 M Na₂CO₃, pH 8.7. An aliquot of this solution was removed for NH₂-groups determination by TNBS titration (final concentration of NH₂-groups, 15 mM or 2.6 mmol total). Methoxy polyethylene glycol succinate (MPEGS 9.6 g, 1.9 mmol) was dissolved in 25 ml of water, degassed, and N-hydroxy(sulfo)succinimide (500 mg, 2.3 mmol) was added, followed by 1 g, 5 mmol of EDC in 2 ml of water. This solution was incubated for 10 min at room temperature and added drop-wise to the solution of poly-L-lysine, final pH 7.7. The mixture was incubated for six hours. The product was purified using ultrafiltration on a cartridge with a cut-off of 100 kD (UFP-100 A/G Technology) to remove unconjugated MPEGS and other reactants.

### Example 2

### Synthesis of PLPEGNTA (lot#20020103)

The product obtained as described in Example 1 (MPEGsuccinyl-poly-L-Lys (m.w. 340000) was succinylated using 10-fold molar excess of succinic anhydride over the concentration of TNBS-reactive free aminogroups in the co-polymer in 0.5 M sodium carbonate pH 8.0, 4 hours room temperature. Succinylated co-polymer (PLPEGSA) was purified using dialysis against water (lot#20020102).

100 mg Lyophilized PLPEGSA was dissolved in 2 ml water at 28 mmol succinate/ml, treated with 30 mg ethyl-diaminopropyl carbodiimide (EDC) in the presence of 20 mg Sulfo-NHS for 10 min at room temperature. A solution of activated PLPEGSA was added to a 10 fold molar excess solution of N,N-Bis(carboxymethyl)-L-lysine Hydrate (BCMLys) in 1 ml sodium bicarbonate, pH 8.7, final pH 7.6, incubated 24 hours at 4° C. The resultant product PLPEGNTA (lot#20020103) was purified using ultrafiltration on a YM50 membrane (Amicon) by diluting to 100 ml and concentrating to 5 ml volume four times. A solution of PLPEGSA was used as a control in further experiments (lot#20020102).

### Example 3

### Synthesis of PLPEGNTANi (lot#20020104)

A solution of product PLPEGNTA was dialyzed against 1 L of 10 mM Ni acetate/20 mM citric acid, pH 6 for 24 hours at 4° C. and purified by dialyzing against 2 L water (2 changes). Binding of Ni was measured by spectrophotometry at 625 nm using Ni-citrate as a standard.

### Example 4

### Synthesis of PLPEGNTAZn (lot#20020105)

A solution of PLPEGNTA was dialyzed against 1 L of 10 mM Zn acetate/20 mM citric acid pH 6 for 24 hours at 4° C., and purified by dialyzing against 2 L water (2 changes). Binding of Zn was measured by using elemental analysis.

### Example 5

### Synthesis of 40PLPEG5371DA (lot#20070927)

One g of 40PL (Sigma P3995; lot# 085K5102; 1 g was found to contain 2.5 mmol NH₂ by TNBS assay according to Spadaro et. al. Anal Biochem, vo196, p 317-321) was dissolved in 50 ml of 200 mM HEPES. 3.5 g of MPEGSuccinate (0.7 mmol; Mw=SkDa; Sunbio; lot# CISA-005-07024) in 25 ml of 10 mM MES pH=4.7 was activated by adding 175 mg of NESS (mw=217.14; 0.8 mmol, followed by 350 mg EDC (mw=191.71; 1.8 mmol). Activation was allowed to proceed for 20 minutes. The activated MPEGSuccinate was added to 40PL solution and allowed to react. After 2 hrs, additional 3.5 g of MPEGSuccinate was activated and added as above and stirred overnight. The next day amino group was measured by TNBS and found to be 1.5 mmol indicating 40% saturation of amino group. The sample was lyophilized (13 g) without cleaning and stored at 4° C. for later use. The lyophilized sample was dissolved in 37 ml water, 2 g Succinic anhydride (SA, 20 mmol) was added, 200 ul TEA was added followed by titration (200 ul at a time) to pH 7.5-8.0 using 10M NaOH. The amino group was measured by TNBS by taking 15 ul and diluting to 1 ml (67 fold; giving 0.2 mg/ml equivalent of original PL). No remaining amino group remaining was found. The resulting 40PLPEG537-succinate or 40PLPEG537SA was washed with 20 volumes of water using ultrafiltration cartridge with molecular weight cut off (MWCO) of 100 kDa (UFP-100-E-5A; GE Healthcare). The 40PLPEG537SA was dried and divided into two (2.95 g each). Iminodiacetic acid (IDA; 1.2 g; Mw=133; 9 mmol; Fisher Cat#AC20497) was prepared in 10 ml of 1M HEPES pH 7.35 in separate flask and pH was adjusted to pH 8.0 using 10 N NaOH. One portion of 40PLPEG537SA (2.95 g; 0.9 mmol carboxyl groups) was made up in 10 ml of 10 mM MES pH 4.7 and activated by adding 250 mg of NHSS (mw=217.14; 1.15 mmol, followed by 500 mg EDC (mw=191.71; 2.6 mmol). Activation of 40PLPEG537SA was allowed to proceed and after 20 minutes the activated 40PLPEG537SA was added to the IDA solution. After the reaction, the 40PLPEG5371DA product was washed with 25 volumes of water using ultrafiltration cartridge with molecular weight cut off (MWCO) of 100 kDa (UFP-100-E-5A; GE Healthcare). Total yield after drying is 2.43 g of 40PLPEG371DA (lot#20070927). The molecular diameter of this material was 19 nm as measured by GPC (column 0.78×30 cm; Tosoh G4000WXL; with PBS/15% Acetonitrile mobile phase flowing at 0.6 ml/min).

### Example 6

### Synthesis of 40PLPEG535DADTPA (Lot#20071101A) and 40PLPEG535DADTPAIDA (20071101B)

One g of 40PL (P3995 Sigma lot# 085K5102) was dissolved in 50 ml of 200 mM HEPES Amino group was measured by TNBS assay and was found to be 2.86 mmol NH₂/g. Three grams of MPEGCM (MethoxyPolyEthyleneGlycol-CarboxyMethyl; 1 mmol; Mw=5 kDa; 9.0 mmol; Laysan Bio; lot#108-41; clear in solution) in 17.5 ml of 10 mM MES pH=4.7 was activated by adding 150 mg of NHSS (mw=217.14; 0.7 mmol), followed by 300 mg EDC (mw=191.71; 1.57 mmol). Activation is allowed to proceed for 20 minutes. Total volume of MPEGCM solution at this stage was 18 ml. The activated MPEGCM was added to 40PL solution and allowed to react. After 45 minutes, additional 3 g of MPEGCM was activated and added as above and allowed to react for 2 hrs. Amino group was measured by TNBS and found to be 103 uM giving 28% saturation. Size Exclusion chromatography using TosohG4000WXL column (0.79×30 cm) eluted with phosphate buffered saline (PBS; 11.9 mM phosphate, 137 mM NaCl, 2.7 mMKPO₄, pH 7.4) containing 15% Acetonitrile at a flow rate of 0.6 ml/min showed a retention time of 11.72 min on UV or 12.20 min on RI (18.4 nm). Another 1.5 g was activated and added to reach 35% amino group saturation based on the remaining amino groups as measured (91 uM in 94 ml or 1.82 mmol total) by TNBS. After addition of 1.5 g of MPEG, retention time on UV becomes 11.60 min or 12.10 min on RI or 19 nm. Four grams of DTPA-dianhydride was added and the pH was adjusted continuously to maintain pH between 7 and 8. After 4 hours, the total amino group was measured by TNBS and was found to be not detectable. The reaction mixture containing 40PLPEG535DADTPA was washed with 20 volumes of water using ultrafiltration cartridge with a molecular weight cut off (MWCO) of 100 kDa (UFP-100-E-5A; GE Healthcare) and lyophilized, giving 4.7 g (40PLPEG535DADTPA, lot#20071101A). Half (2.35 g) was saturated with iminodiacetic acid (IDA) as follows: IDA (3 g) was made up to 10 ml of 1M HEPES, the pH was adjusted to 7.5, and made up to 50 ml in 1M HEPES. Half of 40PLPEG535DADTPA was divided into 3 equal portions (1.3 mmol carboxyl each based on stoichiometry) and each (25 ml) made to pH 4.7 with 200 ul 1M MES, pH 4.7, the pH did not go down to 4.7 and therefore 20 ul of 6N HCl was added. This was activated by addition of 2 mmol NHS S (434 mg) and 4.5 mmol EDC (864 mg). After 20 minutes, the activated 40PLPEG535DADTPA was added to IDA above and repeated 2 more times and stirred for 2 hrs. The product (40PLPEG535DADTPAIDA) was washed with 20 volumes of water, filter-sterilized (0.2 um polysulfone filter, Nalgene, Rochester, N.Y.) and lyophilized giving 2.0 g (40PLPEG535DADTPAIDA, lot#20071101B).

### Example 7

### Synthesis of 40PLPEG537DANTA (lot#20080124a)

One g of 40PL (Sigma P3995 lotnumber127K5101; 1 g was found to contain 2.84 mmol NH₂ as measured by TNBS) was dissolved in 50 ml of 200 mM HEPES. Five grams of MPEGCM (1 mmol; Mw=5 kDa; Sigma/Fisher/Fluka; Cat#70718; lot#64748/1) in 10 mM MES pH=4.7 in 25 ml of 60% ethanol (ethanol was needed to completely dissolve MPEG from Fisher/Fluka) was activated by adding 250 mg of NHS (mw=115.09; 2 mmol), followed by 500 mg EDC (mw=191.71; 1.8 mmol). Activation was allowed to proceed for 20 minutes (total volume is 29 ml). The activated MPEGCarboxyl was added to 40PL solution and additional 6 ml of 1M HEPES added to keep pH at about 7. The mixture was allowed to react overnight. The total volume in the morning was 82 ml and pH is 7.04. The amino group was measured by TNBS and found to be 1.74 mmol total indicating 39% saturation of amino group. Succinic Anhydride (2 g) was added and pH adjusted to maintain at around 7.0 for 2 hours using 10 N NaOH (150 ul at a time approx. 4 ml). After 2 hours, the amino group was measured and no remaining amino group was found. Sample was washed with 20 volume changes of water using a 100 kDa MWCO ultrafiltration cartridge (UFP-100-E-5A, GE-Amersham), filter-sterilized (0.2 um polysulfone filter, Nalgene, Rochester, N.Y.) and lyophilized (4.7 g).

One gram of NTA-amine (Nalpha, Nalpha, -Bis(carboxymethyl)-L-Lysine; Mw=262.26+aq, up to 2 mol water and 10% inorganic) or 3.8 mmol was dissolved in 21 ml of 1M HEPES, pH 7.35. Aliquot (10.5 ul) was taken and diluted to 10 ml for total amino group analysis and found to be 1.8 mmol.

40PLPEG538DASA (4.7 g; 1.7 mmol carboxyl) was dissolved in 33 ml of 10 mM MES, followed by addition of 500 mg NHS (mw=115.09; 4.3 mmol), followed by 2 gram EDC (mw=191.71; 10.4 mmol). During the reaction, pH was maintained below 5.5 (pH 5.0) by HCl while stirring After 12 minutes, the activated 40PLPEG538DASA was transferred to NTA-amine solution and pH was adjusted while stirring using 10 N NaOH to 7.0-7.1 (total reaction volume at this stage was 56 ml). After 3 days, 30 ul aliquot was taken, diluted to 10 ml for amino group analysis, and amino group was found to be 0.059 mmol total NH₂. This was washed with 10 volume changes of water using a 100 kDa MWCO ultrafiltration cartridge (UFP-100-E-5A). The sample was lyophilized yielding 3.9 g (40PLPEG537DANTA, lot#20080124a). This material contains 15 nmol amino group/mg.

### Example 8

### Synthesis of 40PLPEG537DANDA from NTA Attached to the Amino Group of Polylysine (lot#20080124b)

One g of 40PL (Sigma P3995 lotnumber127K5101; 1 g contains 2.62 mmol NH₂) was dissolved in 50 ml of 400 mM HEPES. Five g of MPEGCM (1 mmol; Mw=5 kDa; Sigma/Fisher/Fluka; Cat#70718; lot#64748/1) in 20 ml of 10 mM MES pH=4.7 with 60% ethanol (ethanol was needed to completely dissolve PEG from Sigma/Fisher/Fluka) was activated by adding 250 mg of NHS (mw=115.09; 2 mmol), followed by 500 mg EDC (mw=191.71; 1.8 mmol). Activation is allowed to proceed for 20 minutes (total volume is 20 ml). The activated MPEGCM was added to 40PL solution (pH 7.45 before addition). The mixture was allowed to react for 4 hrs, the total volume is 71 ml and pH is 7.14 at the end. The amino group measurement before (2.62 mmol) and after (1.64 mmol) MPEG addition indicated that the PEG saturation of amino group was 37%. Size Exclusion chromatography using TosohG4000WXL column (0.79×30 cm) eluted with phosphate buffered saline (PBS; 11.9 mM phosphate, 137 mM NaCl, 2.7 mMKPO₄, pH 7.4) containing 15% Acetonitrile at a flow rate of 0.6 ml/min showed a retention time of 12.5 min (or approximately 16 nm in diameter). This is the 40PLPEG537DA solution.

NTA (MW=191; 1 g or 5.2 mmol) was neutralized in water with 1 ml of 10N NaOH and buffered with 20 mM MES at pH 4.7 (total volume is 10 ml). This was activated (in 10 ml of 20 mM MES) with 1 g (5.2 mmol) EDC in the presence of 345 mg NHS (mw=115.09; 3 mmol). After 20 minutes this was added to 40PLPEG537DA. The initial pH of 40PLPEG537DA solution was pH 7.14 but goes down to 6.9 after addition of activated NTA. This was adjusted to 7.25 with 300 ul of 10N NaOH and allowed to react overnight (total volume is 82 ml). The next day, the amino group was measured and found to be only slightly decreased. The pH was lowered to 4.7 using HCl, 2 g EDC was added, and after 20 minutes the pH was raised to 7.0 using 10N NaOH. After 2 hours, the process was repeated and after additional 2 hours the total amino group was measured and found to be 0.03 mmol which is compared to 1.63 mmol original amino groups before the reaction. Sample was washed with 20 volume changes of water using a 100 kDa MWCO ultrafiltration cartridge (UFP-100-E-5A; GE-Amersham), filter-sterilized (0.2 um polysulfone filter; Nalgene, Rochester, N.Y.) and lyophilized giving 4.1 g of 40PLPEG537DANDA.

### Example 9

### Synthesis of 20PLPEG570DANTAZn (Lot#20080326)

a) Two g of 20PL (Q4926 SAFC lot# 018K7775; DP=126; 2 g has 4.76 mmol NH2) was dissolved in 25 ml of 1 M HEPES. Amino group was measured by TNBS assay and found to be 4.76 mmol NH₂/g. b) 14 g of MPEGCM (2.8 mmol; SAFC lot#1372618; orange-yellow in solution) in 52 ml of 50% ethanol with 10 mM MES pH4.7 was activated by adding 700 mg of NHS (mw=115.14; 6.09 mmol), followed by 1.4 g EDC (mw=191.71; 7.30 mmol). Activation is allowed to proceed for 20 minutes. c) The activated MPEGCM was added to 20PL solution and allowed to react 2 hours. When amino groups were measured only 46% saturation was found, thus additional MPEGCM was activated and added (1.2 g) and incubated overnight Amino group analysis indicated 71% PEG saturation. d) Size Exclusion chromatography using TosohG4000WXL column (0.79×30 cm) eluted with phosphate buffered saline (PBS; 11.9 mM phosphate, 137 mM NaCl, 2.7 mMKPO₄, pH 7.4) containing 15% Acetonitrile at a flow rate of 0.6 ml/min showed a retention time of 12.75 min by refractive index or approximately 15 nm molecular diameter. e) Succinic Anhydride (5 g; 50 mmol) added and slowly titrated with 10 N NaOH to pH 7.0 while stirring. After 4 hours the amino group was measured and found to be 0 uM. Size Exclusion chromatography using TosohG4000WXL column (0.79×30 cm) eluted with phosphate buffered saline (PBS; 11.9 mM phosphate, 137 mM NaCl, 2.7 mMKPO₄, pH 7.4) containing 15% Acetonitrile at a flow rate of 0.6 ml/min showed a retention time of 12.5 min by UV 220 and 12.6 min by refractive index or approximately 16 nm molecular diameter. The reaction mixture was washed using a 100 kDa MWCO filter cartridge (UFP-100-E-5A; GE-Amersham), filter-sterilized (0.2 um filter polysulfone filter; Nalgene, Rochester, N.Y.), and lyophilized (40PLPEG570SA; 9.9 g; contains 1.36 mmol carboxyl by stoichiometry). f) Two gram of NTA-amine (Nalpha, Nalpha,-Bis(carboxymethyl)-L-Lysine; Mw=262.26+aq, up to 2 mol water/mol and 10% by weight inorganic) or 7.6 mmol was dissolved in 40 ml of 1M HEPES. Actual amino group measurement indicated 4.28 mmol NTA-amine. g) 40PLPEG570SA (9.9 g; 1.36 nmol Carboxyl) was dissolved in 72 ml of 20 mM MES, and 500 mg NHS (mw=115.09; 4.3 mmol but with water so perhaps 3) was added, followed by 2 gram EDC (mw=191.71; 10.4 mmol). The pH goes up slowly the 20 minute reaction but the pH was maintained below 5.5 by HCl. This solution was added to NTA-amine solution. After 2 hours, amino group analysis indicated that total NTA-amine was down to 3.08 mmol indicating that 1.2 mmol NTA was incorporated into the carrier. h) The reaction mixture was washed with 10 volumes of water using a 100 kDa MWCO filter cartridge (UFP-100-E-5A; GE-Amersham), filter-sterilized (0.2 um polysulfone filter; Nalgene, Rochester, N.Y.), and lyophilized (9.0 g; 20PLPEG570DANTA; Lot#20080326). Analysis by Size Exclusion chromatography using TosohG4000WXL column (0.79×30 cm) eluted with phosphate buffered saline (PBS; 11.9 mM phosphate, 137 mM NaCl, 2.7 mMKPO₄, pH 7.4) containing 15% Acetonitrile at a flow rate of 0.6 ml/min showed a retention time of 12.36 min by refractive index or 12.1 min by UV220 nm or approximately 17.5 nm molecular diameter.

### Example 10

### Synthesis of 20PLPEG550DADTPANTA (Lot#20080411)

a) 1 mL or 0.4 g equivalent of 20PL (Q4926 SAFC lot# 018K7775; DP=126; 0.4 g was found to contain 0.895 mmol NH₂ by TNBS) was dissolved in 5 ml of 1 M HEPES. This is the 20PL solution. b) In a separate container, 2.5 g MPEG was activated in 20 mM MES pH=4.7 (35 ml) by adding 125 mg of NHS (mw=115.14; 1.09 mmol) and 500 mg EDC (mw=191.71; 2.60 mmol) while stirring. Activation was allowed to proceed for 20 minutes and the activated MPEGCM was added directly to 20PL solution in step a. The pH of the reaction mixture was adjusted to pH 7.1 slowly with 10N NaOH one drop at a time, and allowed to react for overnight. Amino group analysis by TNBS showed 0.464 mmol amino groups remains, indicating 50% PEG saturation. This is the 20PLPEG550DA solution. c) Size Exclusion chromatography using TosohG4000WXL column (0.79×30 cm) eluted with phosphate buffered saline (PBS; 11.9 mM phosphate, 137 mM NaCl, 2.7 mMKPO₄, pH 7.4) containing 15% Acetonitrile at a flow rate of 0.6 ml/min showed a retention time of 12.8 min in refractive index detector or approximately 14.4 nm molecular diameter. d) Diethylenetriaminepentaacetic acid dianhydride (1 gram; FW=357.3; 2.80 mmol) was added and slowly titrated with 10 N NaOH to pH 7.1 and stirred for 2 hours. After 2 hours, amino group measurement by TNBS indicated 0% amino group remains. e) The pH of the solution was adjusted to 7.5 using 10N NaOH to facilitate washing as crystals of un-reacted DTPA remains. The solution was concentrated to 100 ml and washed with 15 changes of water using a 100,000 MWCO ultrafiltration cartridge (UFP-100-E-5A; GE-Amersham) and lyophilized. Size Exclusion chromatography using TosohG4000WXL column (0.79×30 cm) eluted with phosphate buffered saline (PBS; 11.9 mM phosphate, 137 mM NaCl, 2.7 mMKPO₄, pH 7.4) containing 15% Acetonitrile at a flow rate of 0.6 ml/min showed a retention time of 12.7 min in a refractive index detector or approximately 15.04 nm molecular. f) 2 gram of NTA-amine (Nalpha, Nalpha, -Bis(carboxymethyl)-L-Lysine; Mw=262.26+50% impurity, up to 2 mol water and 10% inorganic) or ˜4 mmol amino groups was dissolved in 10 ml of 1M HEPES. Amino group analysis by TNBS indicated that the NTA-amine solution contains 3.4 mmol amino groups. g) 20PLPEG550DADTPA (0.70 mmol carboxyl) was dissolved in 10 ml of 20 mM MES, 140 mg NHS (mw=115.09; 1.2 mmol) was added, followed by 560 mg EDC (mw=191.71; 2.9 mmol). The pH goes up slowly but maintained below 5.5 by HCl. This solution was added to NTA solution and the pH was adjusted to pH 7.1 with 10N NaOH. After 2 hours, amino group analysis showed a total of 3.2 mmol amino groups remains, indicating that 0.2 mmol of NTA-amine was incorporated to 0.8 mg carrier. h) The solution was concentrated to 100 ml and washed with 15 changes of water using a 100,000 MWCO ultrafiltration cartridge (UFP-100-E-5A; GE-Amersham), filter-sterilized (0.2 um polysulfone filter, Nalgene, Rochester, N.Y.) and lyophilized yielding 0.5 g (20PLPEG550DADTPANTA; Lot#20080411). Analysis by Size Exclusion chromatography using TosohG4000WXL column (0.79×30 cm) eluted with phosphate buffered saline (PBS; 11.9 mM phosphate, 137 mM NaCl, 2.7 mMKPO₄, pH 7.4) containing 15% Acetonitrile at a flow rate of 0.6 ml/min showed a retention time of 12.7 min in a refractive index detector showing approximately 15.04 nm molecular diameter.

### Example 11

### Synthesis of 20PLPEG1055DANTA (Lot#20080416)

a) 5 mL or 2 g equivalent of 20PL (Q4926 SAFC lot# 018K7775; DP=126; 2 g was found to contain 4.60 mmol NH2 by TNBS) was dissolved in 25 ml of 1 M HEPES. This is the 20PL solution. b) In a separate container, 20 g of MPEGCM (Mw=10 kDa; 2.0 mmol; SunBright; ME-100HS; lot#M62503; clear solution) was dissolved in 60 ml of 80% ethanol with 20 mM MES pH=4.7 (1200 ul of 1M MES added to 60 ml), 500 mg of NHS (mw=115.14; 4.35 mmol) was added, once dissolved 2.0 g EDC (mw=191.71; 10.43 mmol) was added while stirring. Activation was allowed to proceed for 20 minutes and the activated MPEGCM was added directly to 20PL solution in step a. The pH was adjusted to pH 7.1 slowly with 10N NaOH one drop at a time, and allowed to react for 2 hours. Amino group analysis showed 1.92 mmol remains indicating 58% MPEG saturation. This is the 20PLPEG1055DA solution. c) Size Exclusion chromatography using TosohG4000WXL column (0.79×30 cm) eluted with phosphate buffered saline (PBS; 11.9 mM phosphate, 137 mM NaCl, 2.7 mMKPO₄, pH 7.4) containing 15% Acetonitrile at a flow rate of 0.6 ml/min showed a retention time of 11.7 min (or approximately 22.2 nm molecular diameter) and also showing 95% incorporation of MPEG. d) Succinic Anhydride (2 g; 20 mmol) was added followed by 200 uL TEA. The reaction was slowly titrated with 10 N NaOH to pH 7.1 and stirred for 4 hours. The amino groups was measured and found to be 0 umol. Size Exclusion chromatography using TosohG4000WXL column (0.79×30 cm) eluted with phosphate buffered saline (PBS; 11.9 mM phosphate, 137 mM NaCl, 2.7 mMKPO₄, pH 7.4) containing 15% Acetonitrile at a flow rate of 0.6 ml/min showed a retention time is 11.7 min or 21 nm molecular diameter. The reaction mixture was washed with 15 volumes of water using a 100,000 MWCO ultrafiltration cartridge (UFP-100-E-5A; GE-Amersham) and lyophilized (13.1 g). e) 2 gram of NTA-amine (Nalpha,Nalpha, -Bis(carboxymethyl)-L-Lysine; Mw=262.26+50% impurity, up to 2 mol water and 10% inorganic) or ˜4 mmol was dissolved in 10 ml of 1M HEPES. Twenty ml of 0.5M ZnCl was added to the NTA-amine and adjusted to pH7.1 with 10N NaOH. The solution was centrifuge and supernatant was collected and total amino group was determined by TNBS. The TNBS measurement indicated total amino group of 4.80 mmol. f) 20PLPEG1055DASA (3.1 g or 0.40 mmol carboxyl) was dissolve in 15 ml of 20 mM MES, 115 mg NHS (Mw=115.09; 1 mmol) was added, followed by 500 mg EDC (mw=191.71; 2.6 mmol). The pH goes up slowly but was maintained to 4.7 by HCl. After 20 minutes, 20PLPEG1055DASA solution was added to NTA-amine supernatant and pH was adjusted to 7.1 using 10N NaOH. After 2 hours, total amino group was measured by TNBS and found to be 4.0 mmol indicating 0.80 mmol NTA-amine was incorporated to 3.1 g carrier. g) To remove Zinc, 2 g of NTA (Nitrilotriacetic acid; Mw=191.14) or 10 mmol was added to solution in f and adjusted to pH 7.0 with 10N NaOH, followed by 10 ml of Imidazole (5M) and pH goes up to 8. h) The 20PLPEG1055DA-NTA was washed with 10 volumes of water using a 100,000 MWCO ultrafiltration cartridge (UFP-100-E-5A; GE-Amersham). Sample 20PLPEG1055DANTA was filter-sterilized (0.2 um polysulfone filter; Nalgene, Rochester, N.Y.) and lyophilized (2.60 g; 20PLPEG1055DANTA; Lot#20080416). Ten mg/ml of 20PLPEG1055DANTA was analyzed by Size Exclusion chromatography using TosohG4000WXL column (0.79×30 cm) eluted with phosphate buffered saline (PBS; 11.9 mM phosphate, 137 mM NaCl, 2.7 mMKPO₄, pH 7.4) containing 15% Acetonitrile at a flow rate of 0.6 ml/min. The retention time was found to be 11.7 min using UV detector or 11.9 min using refractive index detector (or approximately a 20.6 nm molecular diameter). 1 mg/ml was analyzed by TNBS and contain 0+/−5 uM NH2 or 0 nmol/mg.

### Example 12

### Synthesis of 20PLPEG1055DAPEI4NTAZn (Lot#20080421a)

a) 5 mL or 2 g equivalent of 20PL (Q4926 SAFC lot# 018K7775; DP=126; 2 g was found to contain 4.60 mmol NH2 by TNBS analysis) was dissolved in 25 ml of 1 M HEPES. This is the 20PL solution. b) In a separate container, 20 g of MPEGCM (Mw=10 kDa; 2.0 mmol; SunBright; ME-100HS; lot#M62503; clean in sola) was dissolved in 60 ml of 80% ethanol with 20 mM MES pH=4.7 (1.2 ml of 1M MES added to 60 ml), 500 mg of NHS (mw=115.14; 4.35 mmol) was added, once dissolved 2.0 g EDC (mw=191.71; 10.43 mmol) was added while stirring. Activation was allowed to proceed for 20 minutes and the activated MPEGCM was added directly to 20PL solution in a. The pH was adjusted to slowly to 7.1 using 10N NaOH one drop at a time, and allowed to react for 2 hours. After 2 hours, amino group analysis by TNBS indicated a total of 1.92 mmol remains indicating 58% MPEG saturation. This is the 20PLPEG1055DA solution. c) Size Exclusion chromatography using TosohG4000WXL column (0.79×30 cm) eluted with phosphate buffered saline (PBS; 11.9 mM phosphate, 137 mM NaCl, 2.7 mMKPO₄, pH 7.4) containing 15% Acetonitrile at a flow rate of 0.6 ml/min showed a retention time of 11.7 min (or approximately 22.2 nm molecular diameter) and also showing about 95% incorporation of PEG. d) Succinic Anhydride (2 g; 20 mmol) was added to the 20PLPEG1055DA solution, and followed by 200 uL TEA. The reaction was slowly titrated with 10 N NaOH to pH 7.1 and stirred for 4 hours. After 4 hours, no remaining amino group was detectable by TNBS analysis. Size Exclusion chromatography using TosohG4000WXL column (0.79×30 cm) eluted with phosphate buffered saline (PBS; 11.9 mM phosphate, 137 mM NaCl, 2.7 mMKPO₄, pH 7.4) containing 15% Acetonitrile at a flow rate of 0.6 ml/min showed a retention time was 11.7 min using refractive index detector (or approximately 22.2 nm molecular diameter). The 20PLPEG1040DASA product was washed with 15 volumes of water using a 100 kDa MWCO ultrafiltration cartridge (UFP-100-E-5A; GE-Amersham) and lyophilized giving 13.1 g, e) 25 ml PEI4 (Branched Polyethyleneimine; Mw=400Da; Sigma Chem. Co. St Luis Mo.) was dissolved in 25 ml of 1M HEPES and the pH was adjusted to pH7.4 using about 40 mL of 6N HCl. f) 20PLPEG1055DASA (6.9 g; 1.0 mmol carboxyl) was dissolved in 30 ml of 20 mM MES, 230 mg NHS (mw=115.09; 2 mmol) was added, followed by 1.0 g EDC (mw=191.71; 5.2 mmol). The pH goes up slowly but maintained to 4.7 by adding HCl. After 20 minutes, this solution was added to solution in step e. After 2 hours, the reaction mixture was washed with 15 volumes of water using a 100 kDa MWCO ultrafiltration cartridge (UFP-100-E-5A; GE-Amersham and lyophilized) and lyophilized giving 6.0 g of 20PLPEG1055DAPEI. Analysis by Size Exclusion chromatography using TosohG4000WXL column (0.79×30 cm) eluted with phosphate buffered saline (PBS; 11.9 mM phosphate, 137 mM NaCl, 2.7 mMKPO₄, pH 7.4) containing 15% Acetonitrile at a flow rate of 0.6 ml/min showed a retention time of 11.6 min (or approximately 23.1 nm molecular diameter). g) The amino group content of 20PLPEG1055DAPEI4 was measure by TNBS and found to be 0.186 umol NH₂/mg. 20PLPEG1055DAPEI4 (2 g with 0.37 mmol amino) was dissolved in 30 ml of 1 M HEPES and Succinic Anhydride (2 g; MW=100.07) was added. The reaction was slowly titrated with 10 N NaOH to pH 7.1 and stirred for 2 hours. After 2 hours, the total amino groups was measured by TNBS and found to be 0 umol. The reaction mixture was washed with 15 volumes of water using a 100 kDa MWCO ultrafiltration cartridge (UFP-100-E-5A; GE-Amersham) and lyophilized giving1.9 g of 20PLPEG1055DAPEI4SA. h) NTA-amine (2 g; Nalpha,Nalpha,-Bis(carboxymethyl)-L-Lysine; Mw=262.26+50% impurity, up to 2 mol water and 10% inorganic) equivalent to ˜4 mmol was dissolved in 10 ml of 1M HEPES. Seventeen ml of 0.5M ZnCl was added to the NTA-amine and adjusted to pH7.1 with 10N NaOH. The solution was centrifuged, supernatant was collected, and total amino group was determined by TNBS. Actual amino group measurement by TNBS indicated that there was a total of 3.07 mmol in the supernatant.) 20PLPEG1055DAPEI4SA (1.9 g; theoretical primary amine is 0.35 mmol with another 0.18 secondary amine not detected by TNBS which were all converted to carboxyl in step g) was activated by dissolving it in 20 ml of 20 mM MES, adding 150 mg NHS (mw=115.09; 1.3 mmol), followed by adding 600 mg EDC (mw=191.71; 3.1 mmol, pH is maintained to below 5.4. After 20 minutes, activated 20PLPEG1055DAPEI4SA was added to 10 ml NTA supernatant in h and the pH of the solution was adjusted to 7.1 with 10N NaOH. After 2 hours, the pH was adjusted to =pH5 with 6N HCl and 600 mg EDC (mw=191.71; 3.1 mmol) was further added. After 20 minutes reaction, the pH was adjusted back to 7.1. After 1 hour, total amino group amino group was measured by TNBS and found to be 1.74 mmol indicating 1.33 mmol of NTA-amine was incorporated into 1.9 g carrier. The reaction mixture was washed with 15 volumes of water using a 100 kDa MWCO ultrafiltration cartridge (UFP-100-E-5A; GE-Amersham), filter-sterilized (0.2 um polysulfone filter; Nalgene; Rochester, N.Y.) and lyophilized giving 1.96 g of 20PLPEG1055DAPEI4NTAZn (lot#20080421a). j) Size Exclusion chromatography using TosohG4000WXL column (0.79×30 cm) eluted with phosphate buffered saline (PBS; 11.9 mM phosphate, 137 mM NaCl, 2.7 mMKPO₄, pH 7.4) containing 15% Acetonitrile at a flow rate of 0.6 ml/min showed a retention time of 11.9 min or approximately 20.0 nm molecular diameter. One mg/ml was analyzed by TNBS and contains 0 nmol/mg. Note: This carrier (Lot#20080421a) does not pick up any additional zinc, thus, zinc saturation is maintained during synthesis.

### Example 13

### Synthesis of 20PLPEG1055DAPEI8NTA (Lot#20080421b)

a) 5 mL or 2 g equivalent of 20PL (Q4926 SAFC lot# 018K7775; DP=126; 2 g was found to contain 4.60 mmol NH2 as determined by TNBS analysis) was dissolved in 25 ml of 1 M HEPES. This is the 20PL solution. b) In a separate container, 20 g of MPEGCM (Mw=10 kDa; 2.0 mmol; SunBright; ME-100HS; lot#M62503; clean in sola) was dissolved in 60 ml of 80% ethanol with 20 mM MES pH=4.7 (1.2 ml of 1M MES added to 60 ml), 500 mg of NHS (mw=115.14; 4.35 mmol) was added, once dissolved 2.0 g EDC (mw=191.71; 10.43 mmol) was added while stirring. Activation was allowed to proceed for 20 minutes and the activated MPEGCM was added directly to 20PL solution in step a. The pH was adjusted to pH 7.1 slowly with 10N NaOH one drop at a time, and allowed to react for 2 hours. After 2 hours, amino group analysis by TNBS indicated that 1.92 mmol total amino group remains indicating 58% MPEG saturation. This is the 20PLPEG1055DA solution. c) Size Exclusion chromatography using TosohG4000WXL column (0.79×30 cm) eluted with phosphate buffered saline (PBS; 11.9 mM phosphate, 137 mM NaCl, 2.7 mMKPO₄, pH 7.4) containing 15% Acetonitrile at a flow rate of 0.6 ml/min showed a retention time of 11.7 min or approximately 22.2 nm molecular diameter and also showing 95% incorporation of MPEG. d) Succinic Anhydride (2 g; 20 mmol) was added to the 20PLPEG1055DA solution, followed by 200 uL TEA. The reaction was slowly titrated with 10 N NaOH to pH 7.1 and stirred for 4 hours. After 4 hours, amino groups was measured and found to be 0 umol. Size Exclusion chromatography using TosohG4000WXL column (0.79×30 cm) eluted with phosphate buffered saline (PBS; 11.9 mM phosphate, 137 mM NaCl, 2.7 mMKPO₄, pH 7.4) containing 15% Acetonitrile at a flow rate of 0.6 ml/min showed a retention time was 11.7 min using refractive index detector (or approximately 22.2 nm molecular diameter). The resulting 20PLPEG1040DASA was washed with 15 volumes of water using a 100 kDa MWCO ultrafiltration cartridge (UFP-100-E-5A; GE-Amersham) and lyophilized giving 13.1 g. e) 25 ml PEI8 (Branched Polyethyleneimine; Mw=800 Da; Sigma Chem. Co. St Luis Mo.) was dissolved in 25 ml of 1M HEPES and pH was adjusted to 7.4 using about 40 mL of 6N HCl. f) 20PLPEG1055DASA (3.1 g) was dissolved in 15 ml of 20 mM MES, 115 mg NHS (mw=115.09; 1 mmol) was added, followed by 500 mg EDC (mw=191.71; 2.6 mmol). The pH goes up slowly but maintained to 4.7 by adding HCl. After 20 minutes, this solution was added to solution in step e. After 2 hours, the reaction mixture was washed with 10 volumes of water using a 100,000 MWCO ultrafiltration cartridge (UFP-100-E-5A; GE-Amersham) and lyophilized giving 2.58 g of 20PLPEG1055DAPEI8. Size Exclusion chromatography using TosohG4000WXL column (0.79×30 cm) eluted with phosphate buffered saline (PBS; 11.9 mM phosphate, 137 mM NaCl, 2.7 mMKPO₄, pH 7.4) containing 15% Acetonitrile at a flow rate of 0.6 ml/min showed a retention time of 11.4 min or approximately 24.8 nm molecular diameter. g) The amino group content of 20PLPEG1055DAPEI8 was analyzed using TNBS and found to be 0.296 umol NH2/mg. 20PLPEG1055DAPEI8 (2 g with 0.592 mmol amino) was dissolved in 30 ml of 1 M HEPES and Succinic Anhydride (3 g; MW=100.07; 30 mmol) was added. The reaction was slowly titrated with 10 N NaOH to pH 7.1 and stirred for 2 hours. After 2 hours, the amino groups was measured by TNBS and found to be 0 umol. The reaction mixture was washed with 15 volumes of water using a 100 kDa MWCO ultrafiltration cartridge (UFP-100-E-5A; GE-Amersham) and lyophilized giving 1.8 g of 20PLPEG1055DAPEI8SA. h) Two grams of NTA-amine (Nalpha,Nalpha,-Bis(carboxymethyl)-L-Lysine; Mw=262.26+50% impurity, up to 2 mol water and 10% inorganic) or approximately 4 mmol was dissolved in 10 ml of 1M HEPES, 17 ml of 0.5M ZnCl was added, and pH was adjusted to 7.1 using 10N NaOH. The solution was centrifuged, the resulting supernatant was collected, and total amino group measurement by TNBS. The supernatant contains a total of 6.14 mmol amino group. i) 20PLPEG1055DAPEI8SA (1.8 g; theoretical primary amine is 0.53 mmol with another 0.26 secondary amine not detected by TNBS which were all converted to carboxyl in step g) was dissolved in 20 ml of 20 mM MES, 300 mg NHS (mw=115.09; 2.6 mmol) was added, followed by 1.2 mg EDC (mw=191.71; 6.2 mmol). During the 20 minute reaction, pH is maintained to about 5.4 using HCl. After 20 minutes, the activated 20PLPEG1055DAPEI8SA solution was added to 10 ml NTA-amine supernatant in h and the pH of the solution was adjusted to 7.1 with 10N NaOH. After 2 hours, the pH was adjusted to ˜pH5 with 6N HCl and 1.2 g EDC (mw=191.71; 6.2 mmol) was further added. After 20 minutes reaction, the pH was adjusted back to 7.1. After 1 hour, total amino group was measured by TNBS and found to be 4.08 mmol amino group, indicating that 2.06 mmol of NTA-amine was incorporated into 1.8 g carrier. The reaction mixture was washed with 10 volumes of water using a 100 kDa MWCO ultrafiltration cartridge (UFP-100-E-5A; GE-Amersham), filter-sterilized (0.2 um polysulfone filter; Nalgene; Rochester, N.Y.) and lyophilized (1.55 g; 20PLPEG1055DAPEI8NTAZn; lot#20080421b). j) Size Exclusion chromatography using TosohG4000WXL column (0.79×30 cm) eluted with phosphate buffered saline (PBS; 11.9 mM phosphate, 137 mM NaCl, 2.7 mMKPO₄, pH 7.4) containing 15% Acetonitrile at a flow rate of 0.6 ml/min showed a retention time of 11.4 min or approximately 24.8 nm molecular diameter. TNBS indicated that the product 20PLPEG1055DAPEI8NTAZn has 0 nmol NH2/mg.

### Example 14

### Synthesis of 20PLPEG550DAPEI4NTAZn (lot#20080603c)

a) 15 mL or 6 g equivalent of 20PL (Q4926 SAFC lot# 018K7775; DP=126; 2 g was found to contain 4.72 mmol NH2 by TNBS analysis) was dissolved in 135 ml of 1 M HEPES. This is the 20PL solution. b) In a separate container, 45 g of MPEGCM (Mw=5 kDa; 9.0 mmol; Laysan Bio; 100108-41; clear solution) was dissolved in 150 ml of 80% ethanol with 10 mM MES pH=4.7 (1.5 ml of 1M MES added to 150 ml) and 2.25 g of NHS (mw=115.14; 19.6 mmol) was added, once dissolved 4.5 g EDC (mw=191.71; 23.5 mmol) was added while stirring. Activation was allowed to proceed for 20 minutes and the activated MPEGCM was added directly to 20PL solution in step a. The pH was adjusted to pH 7.1 slowly with 10N NaOH one drop at a time, and allowed to react for 2 hours. After 2 hours, amino group analysis by TNBS indicated that 2.16 mmol remains, indicating 54% MPEG saturation. This is the 20PLPEG550DA solution. c) Size Exclusion chromatography using TosohG4000WXL column (0.79×30 cm) eluted with phosphate buffered saline (PBS; 11.9 mM phosphate, 137 mM NaCl, 2.7 mMKPO₄, pH 7.4) containing 15% Acetonitrile at a flow rate of 0.6 ml/min showed a retention time of 12.8 min (or approximately 14.4 nm molecular diameter) and also showing 95% incorporation of PEG. d) Succinic Anhydride (6 g; 20 mmol) was added to 20PLPEG550DA solution followed by 600 uL TEA. The reaction was slowly titrated with 10 N NaOH to pH 7.1 and stirred for 4 hours. After 4 hours, total amino groups was measured by TNBS and found to be 0 umol. Size Exclusion chromatography using TosohG4000WXL column (0.79×30 cm) eluted with phosphate buffered saline (PBS; 11.9 mM phosphate, 137 mM NaCl, 2.7 mMKPO₄, pH 7.4) containing 15% Acetonitrile at a flow rate of 0.6 ml/min showed retention time of 12.3 min or approximately 17.6 nm molecular diameter. e) The reaction mixture containing 20PLPEG550DASA was concentrated to 400 ml and washed with 15 changes of water in a 100 kDa MWCO ultrafiltration cartridge (UFP-100-E-5A), filter-sterilized (0.2 um polysulfone filter; Nalgene, Rochester, N.Y.) and lyophilized yielding 31 g of 20PLPEG550DASA (Lot#20080523). f) 10 ml PEI4 (Branched Polyethyleneimine; Mw=400Da; Sigma Chem. Co. St Luis Mo.) was dissolved in 20 ml of 1M HEPES and the pH was adjusted to 7.4 using approximately 16 mL of 6N HCl. g) 20PLPEG550DASA (7.7 g; 1.2 mmol carboxyl) was dissolved in 30 ml of 20 mM MES, 260 mg NHS (mw=115.09; 2.3 mmol) was added, followed by 1.2 g EDC (mw=191.71; 6.3 mmol). After 20 minutes, the activated 20PLPEG550DASA was added to the PEI4 solution. After 2 hours, the pH of the reaction mixture was adjusted to 5.0 using 6N HCl and followed by addition of 1.2 g EDC (mw=191.71; 6.3 mmol). After 20 min activation, the pH was adjusted back to pH7.2 with 10N NaOH. h) The reaction mixture containing 20PLPEG550DAPEI4 was concentrated to 100 ml, washed with 10 changes of water using a 100 kDa MWCO ultrafiltration cartridge (UFP-100-E-5A; GE-Amersham), filter-sterilized (0.2 um polysulfone filter, Nalgene, Rochester, N.Y.)) and lyophilized giving 7.2 g (Lot#20080603). i) Size exclusion chromatography using TosohG4000WXL column (0.79×30 cm) eluted with phosphate buffered saline (PBS; 11.9 mM phosphate, 137 mM NaCl, 2.7 mMKPO₄, pH 7.4) containing 15% Acetonitrile at a flow rate of 0.6 ml/min showed a retention time of 11.9 min or approximately 20.6 nm diameter. j) Sample 20PLPEG550DAPEI4 was analyzed by TNBS and found to contain 204 nmol primary amino group/mg. k) 20PLPEG550DAPEI4 (2.0 g; 0.3 mmol amino) was dissolved in 30 ml of 1 M HEPES and Succinic Anhydride (2 g; MW=100.07) was added. The reaction was slowly titrated with 10 N NaOH to pH 7.1 and stirred for 2 hours. After 2 hours, amino group was found to be 0 umol. The reaction mixture containing 20PLPEG550DAPEI4SA was washed with 15 changes of water in 100 kDa MWCO ultrafiltration cartridge (UFP-100-E-5A). 1) Two grams of NTA-amine (Nalpha,Nalpha,-Bis(carboxymethyl)-L-Lysine; Mw=262.26+50% impurity, up to 2 mol water and 10% inorganic) or approximately 4 mmol was dissolved in 5 ml of 1M HEPES. Nine ml of 0.5M ZnCl was added to the NTA and adjusted to pH7.1 with 10N NaOH, followed by centrifugation. The supernatant was collected and the amino group was determined by TNBS analysis. The total amino group in the supernatant was found to be 4.86 mmol. m) 20PLPEG550DAPEI4SA (2 g; 0.5 mmol carboxyl) was activated by dissolving in 20 ml of 20 mM MES, adding 160 mg NHS (mw=115.09; 1.3 mmol), followed by 650 mg EDC (mw=191.71; 3.4 mmol). During the 20 minute activation reaction, pH is maintained to 5.4. After 20 minutes, the activated 20PLPEG550DAPEI4SA was added to 10 ml NTA-amine supernatant and adjusted to pH 7.1 with 10N NaOH. After 2 hours, the pH was adjusted to 5 with 6N HCl and 650 mg EDC (mw=191.71; 3.4 mmol) was added. After 20 minute reaction, the pH was adjusted back to pH7.1. After 1 hour, amino group analysis indicated that 4.86 mmol amino group remains, thus, 0.69 mmol incorporated to 2.0 g carrier. n) Size Exclusion chromatography using TosohG4000WXL column (0.79×30 cm) eluted with phosphate buffered saline (PBS; 11.9 mM phosphate, 137 mM NaCl, 2.7 mMKPO₄, pH 7.4) containing 15% Acetonitrile at a flow rate of 0.6 ml/min showed a retention time of 11.8 min or approximately 21.4 nm molecular diameter showing. o) Sample 20PLPEG550DAPEI4NTAZn was washed with 15 volumes of water (0.2 um polysulfone filter, Nalgene, Rochester, N.Y.), filter sterilized (0.2 um polysulfone filter; Nalgene; Rochester, N.Y.), and lyophilized yielding 1.26 g (Lot#20080603c). Amino group analysis by TNBS indicated that 20PLPEG550DAPEI4NTAZn contain 0.0 nmol amino group/mg.

### Example 15

### Synthesis of 20PLPEG550DAPEI8NTAZn (lot#20080604c)

a) 15 ml or 6 g equivalent of 20PL (Q4926 SAFC lot# 018K7775; DP=126; 2 g was found to contain 4.72 mmol NH₂ by TNBS assay) was dissolved in 135 ml of 1 M HEPES. This is the 20PL solution. b) In a separate container, 45 g of MPEGCM (Mw=5 kDa; 9.0 mmol; Laysan Bio; lot#108-41; clear solution) was dissolved in 150 ml of 80% ethanol with 10 mM MES pH=4.7 (1.5 ml of 1M MES added to 150 ml), 2.25 g of NHS (mw=115.14; 19.6 mmol) was added, once dissolved, 4.5 g EDC (mw=191.71; 23.5 mmol) was added while stirring. Activation was allowed to proceed for 20 minutes. The activated MPEGCM was added directly to 20PL solution in step a. The pH was adjusted to pH 7.1 slowly with 10N NaOH one drop at a time, and allowed to react for 2 hours. After 2 hours, amino group analysis by TNBS indicated that 2.16 mmol amino group remains indicating 54% MPEG saturation. This is the 20PLPEG550DA solution. c) Size exclusion chromatography using TosohG4000WXL column (0.79×30 cm) eluted with phosphate buffered saline (PBS; 11.9 mM phosphate, 137 mM NaCl, 2.7 mMKPO₄, pH 7.4) containing 15% Acetonitrile at a flow rate of 0.6 ml/min showed a retention time of 12.8 min or approximately 14.4 nm molecular diameter and also showing 95% incorporation of MPEG. d) Succinic Anhydride (6 g; 20 mmol) was added 20PLPEG550DA solution followed by 600 uL TEA. The reaction was slowly titrated with 10 N NaOH to pH 7.1 and stirred for 4 hours. The amino groups was measured by TNBS and found to be 0 umol. Size exclusion chromatography as above showed retention time of 12.3 min or approximately 17.6 nm diameter after succinylation. e) The reaction mixture containing 20PLPEG550DASA was concentrated to 400 ml and washed with 15 changes of water in a 100,000 MWCO ultrafiltration cartridge (UFP-100-E-5A), filter-sterilized (0.2 um polysulfone filter, Nalgene, Rochester, N.Y.) and lyophilized yielding giving 31 g of 20PLPEG550DASA (Lot#20080523). g) 20 ml PEI8 (Branched Polyethyleneimine; Mw=800Da; Sigma Chem. Co. St Luis Mo.) was dissolved in 20 ml of 1M HEPES and the pH was adjusted to 7.4 using approximately 32 mL of 6N HCl. h) 20PLPEG550DASA (7.7 g; 1.2 mmol carboxyl) was dissolved in 30 ml of 20 mM MES, 260 mg NHS (mw=115.09; 2.3 mmol) was added, followed by 1.2 g EDC (mw=191.71; 6.3 mmol). The pH goes up slowly but maintained below 5.5 using HCL during the 20 minute activation reaction. After 20 minutes, the activated 20PLPEG550DASA was added to solution in step g. After 2 hours, the pH was adjusted to down to 5.0 with 6N HCl, followed by addition of 1.2 g EDC (mw=191.71; 6.3 mmol). After 20 min activation, the pH was adjusted back to pH7.2 with 10N NaOH. i) The reaction mixture containing 20PLPEG550DAPEI8 was concentrated to 100 ml and washed with 15 changes of water in 100 kDa MWCO ultrafiltration cartridge (UFP-100-E-5A), filter-sterilized (0.2 um polysulfone filter, Nalgene, Rochester, N.Y.) and lyophilized yielding 7.6 g of 20PLPEG550DAPEI8 (Lot#20080604). j) Analysis by Size Exclusion chromatography using TosohG4000WXL column (0.79×30 cm) eluted with phosphate buffered saline (PBS; 11.9 mM phosphate, 137 mM NaCl, 2.7 mMKPO₄, pH 7.4) containing 15% Acetonitrile at a flow rate of 0.6 ml/min showed a retention time of 11.7 min or approximately 22.2 nm molecular diameter. The product 20PLPEG550DAPEI8 was analyzed by TNBS and found to contain 304 nmol/mg. k) 20PLPEG550DAPEI8 (2 g; 0.3 mmol carboxyl) was dissolved in 30 ml of 1 M HEPES, succinic anhydride (2 g; MW=100.07) was added, the reaction was slowly titrated with 10 N NaOH to pH 7.1, and stirred for 2 hours. After 2 hours, the total amino groups was measured and found to be 0.0 umol. The reaction mixture containing 20PLPEG550DAPEI8SA was washed with 15 changes of water in a 100 kDa MWCO ultrafiltration cartridge (UFP-100-E-5A) and lyophilized. l) Three gram of NTA-amine (Nalpha,Nalpha, -Bis(carboxymethyl)-L-Lysine; Mw=262.26+50% impurity, up to 2 mol water and 10% inorganic) of approximately 4 mmol was dissolved in 8 ml of 1M HEPES and 14 ml of 0.5M ZnCl was added to the NTA-amine and adjusted to pH7.1 with 10N NaOH. The solution was centrifuged, supernatant was collected, and the total amino group was determined TNBS. Amino group was found to be 3.07 mmol. m) 20PLPEG550DAPEI8SA (2 g; 0.5 mmol carboxyl) was activated by dissolving in 20 ml of 20 mM MES, adding 160 mg NHS (mw=115.09; 1.3 mmol), followed by 650 mg EDC (mw=191.71; 3.4 mmol). During the 20 minute activation reaction, pH was maintained below 5.5. After 20 minutes, the activated 20PLPEG550DAPEI8SA was added to 10 ml NTA supernatant from step 1 and the pH was adjusted to 7.1 with 10N NaOH. After 2 hours, the pH was adjusted to ˜pH5 with 6N HCl and 650 mg EDC (mw=191.71; 3.4 mmol) was added. After 20 minute reactivation reaction, the pH was adjusted back to 7.1. After 1 hour, total amino group was measured by TNBS and found to be 7.85 mmol, indicating that 0.62 mmol NTA was incorporated to 2.0 g carrier. n) Analysis by Size Exclusion chromatography using TosohG4000WXL column (0.79×30 cm) eluted with phosphate buffered saline (PBS; 11.9 mM phosphate, 137 mM NaCl, 2.7 mMKPO₄, pH 7.4) containing 15% Acetonitrile at a flow rate of 0.6 ml/min showed a retention time of 11.7 min or approximately 22.2 nm molecular diameter. o) Sample 20PLPEG550DAPEI8NTAZn was washed with 15 changes of water in a 100 kDa MWCO ultrafiltration cartridge (UFP-100-E-5A), filter-sterilized (0.2 um polysulfone filter, Nalgene, Rochester, N.Y.) and lyophilized yielding 1.27 g of 20PLPEG550DAPEI8NTAZn (lot#20080604c). TNBS analysis indicated that 20PLPEG550DAPEI8NTAZn (lot#20080604c) contains 0 nmol/mg primary amino groups.

### Example 16

### Synthesis of 20PLPEG550DAPEI12NTAZn (lot#20080605c)

a) 15 mL or 6 g equivalent of 20PL (Q4926 SAFC lot# 018K7775; DP=126; 2 g was found to contain 4.72 mmol NH₂ as determined by TNBS assay) was dissolved in 135 ml of 1 M HEPES. This is the 20PL solution. b) In a separate container, 45 g of MPEGCM (Mw=5 kDa; 9.0 mmol; Laysan Bio; lot#108-41; clear in solution) was dissolved in 150 ml of 80% ethanol with 10 mM MES pH=4.7 (1.5 ml of 1M MES added to 150 ml), 2.25 g of NHS (mw=115.14; 19.6 mmol) was added, once dissolved 4.5 g EDC (mw=191.71; 23.5 mmol) was added while stirring. Activation was allowed to proceed for 20 minutes and the activated MPEGCM was added directly to 20PL solution in step a. The pH was adjusted to pH 7.1 slowly with 10N NaOH one drop at a time, and allowed to react for 2 hours. Amino group analysis by TNBS indicated a total amino group of 2.16 mmol indicating 54% MPEG saturation. This is the 20PLPEG550DA solution. c) Size Exclusion chromatography of 20PLPEG550DA using TosohG4000WXL column (0.79×30 cm) eluted with phosphate buffered saline (PBS; 11.9 mM phosphate, 137 mM NaCl, 2.7 mMKPO₄, pH 7.4) containing 15% Acetonitrile at a flow rate of 0.6 ml/min showed a retention time of 12.8 min or approximately 14.4 nm molecular diameter and also showing 95% incorporation of PEG. d) Succinic Anhydride (6 g; 20 mmol) was added to 20PLPEG550DA and followed by 600 uL TEA. The reaction was slowly titrated with 10 N NaOH to pH 7.1 and stirred for 4 hours. After 4 hours, the total amino group was measured by TNBS and was found to be 0 umol. The product 20PLPEG550DASA was analyzed by Size exclusion chromatography as above and found to have retention time of 12.3 min or approximately 17.6 nm in diameter. e) The reaction mixture containing 20PLPEG550DASA was concentrated to 400 ml and washed with 15 changes of water in 100 kDa MWCO ultrafiltration cartridge (UFP-100-E-5A), filter-sterilized (0.2 um polysulfone filter, Nalgene, Rochester, N.Y.), and lyophilized yielding 31 g of 20PLPEG550DASA (lot#20080523). g) 50 ml PEI12 (Branched Polyethyleneimine; Mw=1200Da; Sigma Chem. Co. St Luis Mo.) was dissolved in 20 ml of 1M HEPES, pH was adjusted to 7.4 using approximately 45 mL of 6N HCl. h) 20PLPEG550DASA (7.7 g; 1.2 mmol carboxyl) was dissolved in 30 ml of 20 mM MES, 260 mg NHS (mw=115.09; 2.3 mmol) was added, followed by 1.2 g EDC (mw=191.71; 6.3 mmol). The pH goes up slowly but maintained to at below 5.5 using HCl. After 20 minutes, the activated 20PLPEG550DASA was added to solution in step g. After 2 hours, the pH was adjusted to pH 5.0 with 6N HCl and followed by addition of 1.2 g EDC (mw=191.71; 6.3 mmol). After 20 min activation, the pH was adjusted back 7.2 with 10N NaOH. i) After 2 hours, the reaction mixture containing 20PLPEG550DAPEI12 was concentrated to 100 ml and washed with 15 changes of water in a 100 kDa MWCO ultrafiltration cartridge (UFP-100-E-5A), filter-sterilized (0.2 um polysulfone filter, Nalgene, Rochester, N.Y.) and lyophilized yielding 7.8 g of 20PLPEG550DAPEI12 (lot#20080605). j) Analysis of 20PLPEG550DAPEI12 by size exclusion chromatography using TosohG4000WXL column (0.79×30 cm) eluted with phosphate buffered saline (PBS; 11.9 mM phosphate, 137 mM NaCl, 2.7 mMKPO₄, pH 7.4) containing 15% Acetonitrile at a flow rate of 0.6 ml/min showed a retention time of 11.7 min or approximately 22.2 nm molecular diameter. TNBS analysis indicated that 20PLPEG550DAPEI12 contains 448 nmol amino group/mg. k) 20PLPEG550DAPEI12 (2 g; 0.3 mmol carboxyl) was dissolved in 30 ml of 1M HEPES and succinic anhydride (2 g; MW=100.07) was added. The reaction mixture was slowly titrated with 10 N NaOH to pH 7.1 and stirred for 2 hours. After 2 hours the total amino group was measured by TNBS and found to be 0 umol. The reaction mixture containing 20PLPEG550DAPEI12SA was washed with 15 changes of water in 100,000 MWCO ultrafiltration cartridge (UFP-100-E-5A), filter-sterilized (0.2 um polysulfone filter, Nalgene, Rochester, N.Y.) and lyophilized. 1) Four gram of NTA-amine (Nalpha,Nalpha, -Bis(carboxymethyl)-L-Lysine; Mw=262.26+50% impurity, up to 2 mol water and 10% inorganic) of approximately 4 mmol of was dissolved in 10 ml of 1M HEPES, 18 ml of 0.5M ZnCl was added, and the pH was adjusted to 7.1 with 10N NaOH. The NTA amine solution was centrifuged, supernatant was collected, and the total amino group in the supernatant was determined by TNBS and indicated that there was 3.07 mmol total amino group. m) 20PLPEG550DAPEI12SA (2 g; 0.5 mmol carboxyl for primary amine succinate) was dissolved in 20 ml of 20 mM MES, 160 mg NHS (mw=115.09; 1.3 mmol) was added, followed by 650 mg EDC (mw=191.71; 3.4 mmol). During the 20 minute activation reaction, pH was maintained below 5.5. After 20 minutes, activated 20PLPEG550DAPEI12SA solution was added to 10 ml NTA supernatant and the pH was adjusted to pH 7.1 using 10N NaOH. After 2 hours, the pH was adjusted back to 5.5 using 6N HCl, and 650 mg EDC (mw=191.71; 3.4 mmol) was added. After 20 minute reactivation reaction, the pH was adjusted back to pH7.1 with 10N NaOH. After 1 hour, amino group analysis by TNBS indicated that 2.15 mmol amino groups remains, indicating that 0.92 mmol was incorporated to 2.0 g carrier. Sample 20PLPEG550DAPEI12NTAZn was washed with 15 changes of water in 100,000 MWCO ultrafiltration cartridge (UFP-100-E-5A), filter-sterilized (0.2 um polysulfone filter, Nalgene, Rochester, N.Y.) and lyophilized yielding 2.5 g (lot#20080605c). n) Analysis of 20PLPEG550DAPEI12NTAZn by size exclusion chromatography using TosohG4000WXL column (0.79×30 cm) eluted with phosphate buffered saline (PBS; 11.9 mM phosphate, 137 mM NaCl, 2.7 mMKPO₄, pH 7.4) containing 15% Acetonitrile at a flow rate of 0.6 ml/min showed a retention time of 11.9 min or approximately 20.6 nm molecular diameter. TNBS analysis indicated that 20PLPEG550DAPEI12NTAZn contains 0 nmol amino group/mg.

### Example 17

### Synthesis of 18PEIPEG1030DANTAZn (Lot#20080804b)

a) 3.2 g of 18PEI (408700 Aldrich lot#07326LH; DP=126) was and titrated to pH7.4 with 2.9 ml of 6N HCl and made up to 32 ml of 1 M HEPES. Primary amino group by TNBS analysis was found to be 10.24 mmol NH₂/3.22 g. This is the 18PEI solution. b) In a separate container, 15 g of MPEGCM (Mw=10 kDa; 1.45 mmol; Laysan; lot#108-108; clear in solution) was dissolved in 45 ml of 80% ethanol with 10 mM MES pH=4.7 (600 ul of 1M MES added to 60 ml), 375 mg of NHS (mw=115.14; 3.26 mmol) was added, once dissolved 1.5 g EDC (mw=191.71; 7.82 mmol) was added while stirring. Activation was allowed to proceed for 20 minutes and the activated MPEGCM was added directly to 18PEI solution in step a. The pH was adjusted to pH 7.1 slowly with 10N NaOH one drop at a time, and allowed to react for 2 hours. After 2 hours, the pH was adjusted back to 5.5 with 6N HCl and 1.5 g EDC (mw=191.71; 7.82 mmol) was added. After a 20 minute reaction, the pH was adjusted back to pH7.1. After 2 hours, amino group analysis by TNBS indicated 7.53 mmol amino group remains, indicating 26% PEG saturation. This is the 18PEIPEG1030DA solution. c) The reaction mixture containing 18PEIPEG1030DA was washed with 10 volumes of 80% ethanol using a 3 kDa MWCO filter cartridge (UFP-10-E-5A; GE-Amersham), filter-sterilized (0.2 um polysulfone filter, Nalgene, Rochester, N.Y.), and lyophilized (7.76 g; 18PEIPEG1030DA; Lot#20080804). Analysis of 18PEIPEG1030DA by Size Exclusion chromatography using TosohG3000WXL column (0.79×30 cm) eluted with phosphate buffered saline (PBS; 11.9 mM phosphate, 137 mM NaCl, 2.7 mMKPO₄, pH 7.4) containing 15% Acetonitrile at a flow rate of 0.6 ml/min showed a retention time of 9.75 min by UV220 nm or approximately 9.5 nm in diameter. 18PEIPEG1030DA was analyzed by TNBS and found to contain 310 nmol NH₂/mg. d) 18PEIPEG1030DA (1.5 g; 0.47 mmol amino) was dissolved in 30 ml of 1M HEPES, succinic anhydride (2 g; 10 mmol) was added, the solution was slowly titrated with to pH 7.1 using 10 N NaOH, and stirred for 4 hours. After 4 hours, amino groups was measured by TNBS and found to be 0 umol. The reaction mixture containing 18PEIPEG1030DASA was washed with 10 volumes of 80% ethanol using a 100 kDa MWCO filter cartridge (UFP-100-E-5A; GE-Amersham), and concentrated in 80% ethanol and collected. Analysis by Size Exclusion chromatography using TosohG4000WXL column (0.79×30 cm) eluted with phosphate buffered saline (PBS; 11.9 mM phosphate, 137 mM NaCl, 2.7 mMKPO₄, pH 7.4) containing 15% Acetonitrile at a flow rate of 0.6 ml/min showed a retention time of 12.8 min by UV220 nm or approximately 14.3 nm in diameter. e) Half gram of NTA-amine (Nalpha,Nalpha,-Bis(carboxymethyl)-L-Lysine; Mw=262.26+unknown % impurity, up to 2 mol water and 10% inorganic) of about 1.9 mmol was dissolved in 3 ml of 1M HEPES, 3 ml of 0.5M ZnCl was added and the pH was adjusted to pH7.1 with 10N NaOH. The solution was centrifuged, supernatant was collected, and total amino group of supernatant was measured by TNBS and found to be 1.71 mmol. j) 18PEIPEG1030DASA (200 ml from D; 0.5 mmol carboxyl from primary amino by stoichiometry not including secondary or tertiary amine) was buffered with 2 ml of 1M MES, pH 4.7 and activated by adding 230 mg NHS (mw=115.09; 2.0 mmol) followed by 1.15 g EDC (mw=191.71; 6.0 mmol). The pH was maintained below 5.5 using HCl. After 20 minutes the activated 18PEIPEG1030DASA was added to 13 ml of NTA-Zn supernatant in step e and the pH was adjusted to 7.1 using 10N NaOH. The solution was magnetically stirred overnight. The next day, total amino group was measured by TNBS and was found to be 0.47 mmol, indicating that. 1.24, the total amino group incorporated into the carrier is 1.24 mmol. g) The reaction mixture containing 18PEIPEG1030DANTAZn was washed with 10 volumes of 80% ethanol using a 100 kDa MWCO Filter cartridge (UFP-100-E-5A; GE-Amersham) followed by 10 volume of water, filter-sterilized (0.2 um polysulfone filter, Nalgene, Rochester, N.Y.) and lyophilized yielding 1.65 g (18PEIPEG1030DANTAZn; lot#20080804b). Analysis by Size Exclusion chromatography using TosohG4000WXL column (0.79×30 cm) eluted with phosphate buffered saline (PBS; 11.9 mM phosphate, 137 mM NaCl, 2.7 mMKPO₄, pH 7.4) containing 15% Acetonitrile at a flow rate of 0.6 ml/min showed a retention time of 12.2 min or approximately 19.1 nm in diameter. TNBS analysis indicated that the 18PEIPEG1030DANTAZn has only 2 nmol primary amino group/mg.

It should be noted that the 17 synthesis examples above that used linear and branched polymer backbone represented by polylysine and polyethyleneimine species are not to limit the scope of the invention. The use of other backbone without undue experimentations by those skilled in the arts is inherently disclosed in this specification. The invention include the use of other backbones such as polyaspartic acid, polyglutamic acid, polyserine, polythreonine, polycysteine, polyglycerol, polyallylamine, chitosan, natural saccharides, aminated polysaccharides, aminated oligosaccharides, polyamidoamine, polyacrylic acids, polyalcohols, sulfonated polysaccharides, sulfonated oligosaccharides, carboxylated polysaccharides, carboxylated oligosaccharides, aminocarboxylated polysaccharides, aminocarboxylated oligosaccharides, carboxymethylated polysaccharides, or carboxymethylated oligosaccharides are meant to be disclosed in this specification. Those backbones mentioned above with repeating carboxyl groups can be activated to react with amine containing chelating molecule or modified to by a small molecule spacer to contain amino group and facilitate reaction with carboxyl containing chelating group. Those backbones mentioned above with repeating hydroxyl groups can be reacted chelating molecule or modified to by a small molecule spacer to contain group that will facilitate reaction with chelating group. Those backbones mentioned above with repeating sulfonyl can be reacted chelating molecule or modified by a small molecule spacer to contain group that will facilitate reaction with chelating group or protective group or both.

Again, it should be noted that the 17 synthesis examples above that used linear and branched polymer backbone represented by polylysine and polyethyleneimine species are not to limit the scope of the invention. The invention also include the use of aliphatic backbones such as those within a composition with a general formula [PᵥN_{w}CₓH_{y}O_{z}-] where v is 0-3, w is 0-3, x is 8-48; y is 15-95; z is 1-13. In this case these backbones will have modification that will be receptive to the addition of at least one chelating group and this process is well known to those skilled in the art without undue expermentation. The present disclosure of the invention is also meant to include the use of hydrophobic backbone derived from aliphatic chain or group with at least 10 carbons with a general formula [CH₃(CH)ₓ-] where x is 10-35. These include a fatty acid selected from caprylic acid, Capric acid, Laurie acid, Myristic acid, Palmitic acid, Stearic acid, Arichidic acid, Behenic acid, and Lignoceric acid. In another embodiment, the fatty acids is Stearic acid. In another embodiment, the fatty acids is behenic acid. In another embodiment, the fatty acids is lignoceric acid. The present disclosure of the invention is also meant to include the use of hydrophobic backbone derived from polyamino acids and other small hydrophobic molecule such as poly-L-glycine, poly-L-alanine, poly-L-valine, poly-L-leucine, poly-L-isoleucine, poly-L-phenylalanine, poly-L-proline, poly-L-methionine, poly-D-glycine, poly-D-alanine, poly-D-valine, poly-D-leucine, poly-D-isoleucine, poly-D-phenylalanine, poly-D-proline, poly-D-methionine, poly-D/L-glycine, poly-D/L-alanine, poly-D/L-valine, poly-D/L-leucine, poly-D/L-isoleucine, and poly-D/L-phenylalanine, poly-D/L-proline, poly-D/L-methionine, phenyl, naphthyl, cholesterol, vitamin D, and/or vitamin E.

It should also be noted that the 17 synthesis examples above that used a bidentate, a tridentate and a tetradentate chelating molecule represented by species IDA, NTA, and DTPA is not to limit the scope of the invention to these species but rather to show examples of how the invention can be enabled and easily practiced by those skilled in the art. Other chelating moieties can be used using the simple chemistry which is very well known to those skilled in the art. Examples of chelating molecule that can be used without undue experimentation includes 1,4,7,10-tetraazacyclododecane-N,N′,N″,N′″-tetraacetic acid; 1,4,7,10-tetraaza-cyclododecane-N,N′,N″-triacetic acid; 1,4,7-tris(carboxymethyl)-10-(2′-hydroxypropyl)-1,4,7,10-tetraazocyclodecane; 1,4,7-triazacyclonane-N,N′,N″-triacetic acid; 1,4,8,11-tetraazacyclotetra-decane-N,N,N″,N′″-tetraacetic acid; 1,2-diaminocyclohexane-N,N,N′,N′-tetraacetic acid; bis(aminoethanethiol)carboxylic acid; diethylenetriamine-pentaacetic acid (DTPA); ethylenediamine-tetraacetic acid (EDTA); ethyleneglycoltetraacetic acid (EGTA); ethylene-bis(oxyethylene-nitrilo)tetraacetic acid; ethylenedicysteine; Imidodiacetic acid (IDA); N-(hydroxyethyl)ethylenediaminetriacetic acid; nitrilotriacetic acid (NTA); nitrilodiacetic acid (NDA); triethylenetetraamine-hexaacetic acid (TTHA); a bisphosphonate or a polypeptide having the formula: (AₓH_{y})ₚ, wherein A is any amino acid residue, H is histidine, x is an integer from 0-6; y is an integer from 1-6; and p is an integer from 2-6. The bisphosphonate above may be pamidronate, etidronate, alendronate, ibandronate, zoledronate, risendronate, and other derivatives thereof such as a derivative of pamidronate.

It should also be noted that thel7 synthesis examples above that used a transition metal ion represented by species Zinc and Nickel is not intended to limit the scope of the invention to these species but rather to show examples of how the invention can be enabled and easily practiced by those skilled in the art. Other transition metal ions that can be used are Zinc, Nickel, Cobalt, Iron, Manganese, or Copper, without undue experimentations by those skilled in the art.

### Example 18

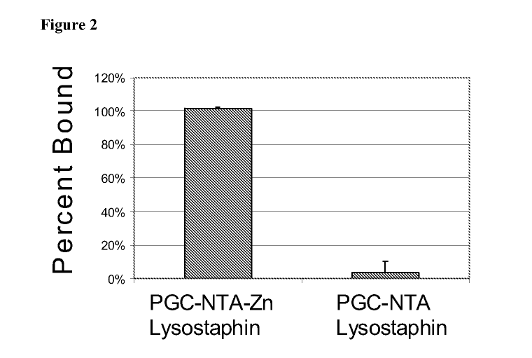

### Binding of a Metallopeptidase to Carrier-NTA-Zn (lot#20020105) but not to Carrier-NTA Without Zinc (lot#20020103) (FIG. 2)

The ability of carrier-NTA-Zn was initially tested at 10% loading. For this experiment, 100 ul of an 8.8 uM molar metallopeptidase (lysostaphin) solution was added to 100 ul of 1.4 uM of carrier-NTA-Zn and 1.4 uM carrier-NTA (control) solutions. These solutions were allowed to incubate for ˜1 hour at RT and filtered through Amicon Ultrafree-MC centrifuge filters (MWCO 100 k) by centrifugation 10,000×g for 10 minutes. The resulting filtrates representing free lysostaphin were quantified at 420 nm after TNBS reaction described in Example 19 below.

### Example 19

### TNBS Assay for Primary Amino Groups

The assay for primary amino groups was adapted from Spadaro et al. (Anal Biochem, vol96, p 317-321) and modified to fit a 96-well plate. Stock borate buffer (2.5×) containing 0.1M sodium tetraborate, pH 9.2, was prepared by stirring overnight at room temperature followed by filtration through 0.2 um filter (0.2 um polysulfone filter, Nalgene, Rochester, N.Y.). Lysine stock standard (2.34 mg/ml) was prepared and kept frozen until use. Prior to use the stock was serially diluted with water 100 fold (23.4 ug/ml or 256 uM primary amino groups); 200 fold (128 uM primary amino groups); 400 fold (64 uM primary amino groups); 800 fold (32 uM primary amino groups); 800 fold (32 uM primary amino groups); and 1600 fold (16 uM primary amino groups). These were plated (150 ul/well) in 96-well plate (Corning transparent flat bottom polystyrene; Fisher) in triplicate including water alone as zero blank. Samples (150 ul) with unknown amounts of primary amino group were also plated (in triplicate) in separate wells. TNBS (1M) was diluted 400 fold using 2.5×borate buffer and 100 ul was added to samples or standards in the 96-well plate. After 30 minutes the absorbance at 420 nm was measured using Chameleon plate reader. The amino groups in the samples were calculated from regression equation of the standard curve (normally close to y=0.005×−0.02; r²=0.999; where y is the absorbance at 420 nm and x is the concentration of primary amino group in uM) ran with the sample.

### Example 20

### Testing of the Ability of Various Carriers to Bind to a Metallopeptidase (Lysostaphin)

Incubation mixtures in triplicate were prepared to determine the ability of various carriers to bind peptides and proteins in general. For 2, 10, 20, 50, 100% loading (weight of protein or load lysostaphin×100/wt of carrier), 250 ul test solutions were prepared in triplicate at appropriate final buffer concentration (10 mM HEPES, pH 7.3 for lysostaphin) containing 0.2 mg/ml test peptide or test proteins, and 10, 2, 1, 0.5, and 0.2 mg/ml Carrier. Control samples without carriers were also prepared in similar manner. Samples and controls were filtered through 100 kDa molecular weight cut off centrifugal membrane filter (Ultracel YM-100; Millipore, Bedford, Mass.) by centrifugation at 14,000×g for 1 minutes. Free lysostaphin in the filtrate was analyzed by TNBS according to Spadaro et al. (Anal Biochem, vol96, p 317-321). The results of these studies are presented in Table 3.

TABLE 3
Summary of binding Properties of some of the PGC-MBs
(Metal Bridge Carriers)

| | | Size | NH2/mg | % Free lysostaphin |
|---|---|---|---|---|
| Lot# | Structure Name* | (nm) | (nmol) | @x % load** |
| 20070927 | 40PLPEG537IDAZn | 19 | 0 | 4.3@50%; 9.5@100% |
| 20071101A | 40PLPEG535DTPAZn | 19 | 0 | 13.5@50%; 32.8@100% |
| 20071101B | 40PLPEG535DTPAIDAZn | 19 | 0 | 4.3@50%; 20.5@100% |
| 20080124a | 40PLPEG539DANTAZn | 20 | 20 | 2@20%; 10@100% |
| 20080124b | 40PLPEG537DANDAZn | 20 | 10 | 44@50%; 46@100 |
| 20080326 | 20PLPEG570DANTAZn | 17 | 0 | 6@50%; 33@100% |
| 20080411 | 20PLPEG550DADTPANTAZn | 15 | 0 | 7@50%; 10@100% |
| 20080416 | 20PLPEG1055DANTAZn | 21 | 0 | 4@50%; 20@100% |
| 20080421a | 20PLPEG1055DAPEI4-NTAZn | 20 | 0 | 2@50%; 7@100% |
| 20080421b | 20PLPEG1055DAPEI8NTAZn | 25 | 0 | 4@50%; 16@100% |
| 20080603c | 20PLPEG550DAPEI4NTAZn | 21 | 0 | 7@50%; 2@100% |
| 20080604c | 20PLPEG550DAPEI8NTAZn | 22 | 0 | 5@50%; 3@100% |
| 20080605c | 20PLPEG550DAPEI12NTAZn | 21 | 0 | 5@50%; 4@100 |
| 20080804b | 18PEIPEG1030DANTAZn | 19 | 2 | 2@50%; 10@100% |

*The names of the structures in Table 3 are convention and adapted for easy identification of the carrier. In the above table the 40PL and 20PL indicates a backbone of 40 kDa and 20 kDa polylysine, respectively. The following PEG537, and PEG535, indicate 5 kDa MPEGSuccinate attached to 37, and 35% of the total epsilon amino groups of polylysine, respectively. The following PEG537DA, PEG539DA, PEG550DA and PEG570DA, indicate 5 kDa MPEGcarboxyl attached to 37, 39, 50 and 70% of the totalepsilon amino groups of polylysine. The PEG1055DA, PEG1040DA, and PEG1030DA indicate 10 kDa MPEGcarboxyl attached to 55, 40, and 30% of the total epsilon amino groups of polylysine. After the PEG portion, the remaining amino groups is further derivatized by chelators such as iminodiacetic acid-Zn (IDAZn), diethylenetriaminepentaacetic acid-Zn (DTPAZn), nitrilotriacetic acid-Zn (NTAZn), nitrilodiacetic acid-Zn (NDAZn) via succinate linker. In some design, the remaining amino groups afterPEG addition were multiplied by attaching 0.4 kDa, 0.8 kDa, or 1.2 kDa polyetheleneimine (PEI4, PEI8, and PEI12 shown in the table) through succinate linker before addition of the chelators as indicated in the table. For the design with - DTPANTA, the four exposed carboxyl groups of DTPA were derivatized with NTA, thus multiplying the number of chelator present per molecule.
**The “x % load” indicates the amount x (weight) of load molecule (lysostaphin) expressed as percent of carrier weight used. The percent free at various level of loading gives us a rough approximation of how well the carrier binds a specific load molecule. This is also use for quality control purpose. The lower the free the tighter the binding and if the amount of free remains low at higher loading, it indicates a high capacity binding. Proper determination Kd and capacity isusually performed by binding study and Scatchard plot (see below and FIGS. 3 to 5).

### Example 21

### The Binding of a Metallopeptidase (Lysostaphin) to the Composition of the Present Invention is Characterized by High Affinity and High Capacity (See FIGS. 3-5)

The dissociation constants or Kds of lysostaphin to some PGC-MB carriers are less than 200 nM with capacity of about 20 lysostaphin molecules per carrier molecule. FIGS. 3-5 show the Scatchard plots (y-axis is bound/free; x-axis is bound; slope is −1/kd; x-intercept is the capacity) with various Kds and capacity of three selected carriers for demonstration purpose. In this particular experiment, 250 ul solutions were prepared in triplicate containing 0 or 50 ug carrier (approximate Mw=300 kDa) with 12.5, 25, 50, 75, 100, 150, and 200 ug of lysostaphin (Mw=26 kDa; AMBI, Lawrence, N.Y.) in 10 mM HEPES, pH 7.35. These correspond to loading of 25%, 50%, 100%, 150%, 200%, 300%, and 400% of the carrier weight, respectively. Samples were incubated for 30 minutes and the bound was filtered out using 100 kDa MWCO cellulose filter (Ultracell YM-100; Millipore, Bedford, Mass.) by centrifugation at 12,000×g for 12 minutes. The filtrate (free lysostaphin) was quantified by TNBS assay (see example 19 above) adapted from Sparado et. al. (Anal. Biochem 1979, 96:317-321). Bound lysostaphin was released by pipetting 250 uL of 0.2M EDTA (prepared in 10 mM HEPES at pH7.3) to the filter followed by centrifugation at 12000×g for 10 minutes. Lysostaphin had been tested and found not to bind to the filter in any significant amount that can compromise the analysis. The total lysostaphin that went through the filter in the absence of the carrier (control) was taken as the total lysostaphin loaded. A calibration curve for the amount of lysostaphin and the corresponding TNBS response was used to determine the amount of lysostaphin and a molecular weight of 26,000 Da was used to determine the molar concentration of lysostaphin. The sample standard curve for quantification of lysostaphin using TNBS gave a linear equation of y=3.447×−0.023 with r²=0.99; where y is absorbance or optical density and x is the concentration of lysostaphin in mg/ml. The bound lysostaphin for each incubation mixture was determined by subtracting the free lysostaphin concentration in the filtrate from the corresponding lysostaphin concentration in the control filtrate. The free lysostaphin is the concentration of lysostaphin in the filtrate coming from incubation mixture with the carrier. The Bound/Free was plotted (y-axis) against Bound concentration (x-axis) to obtain a Scatchard plot. The slope of the plot was determined by regression analysis and used to obtain dissociation constant or Kd which is equal to (−1/slope). The y-intercept is the saturation and to determine the capacity of the carrier the y-intercept was divided by the concentration of carrier (in M) used in the incubation mixture which in this case is 500 nM since the carriers have approximate molecular weight of 350-450 kDa. Plots below show the results of the binding study between lysostaphin and three carriers (20PLPEG550PEI4NTAZn lot#20080603c; 20PLPEG550PEI8NTAZn lot#20080604c; 20PLPEG550PEI12NTAZn lot#20080605c). The density of Zinc increases in order of lot#20080603c<20080604c<20080605c. The Kd decreases in order of 156 nM for lot#20080603c<115 nM for lot#20080604c<99 nM for lot#20080605c. The capacity also increases in the order of 19 lysostaphin/carrier for lot#20080603c<20 lysostaphin/carrier for lot#20080604c<24 lysostaphin/carrier for lot#20080605c.

### Example 22

### The Carriers of the Invention Enhance Metallopeptidase (Lysostaphin) Activity in the Presence or Absence of Serum and With or Without (W/O) Protease Inhibitors (PI)

Table 4 shows carriers loaded with 20% lysostaphin. The carrier structures are as follows: 0124a=40PLPEG539DANTA; 0326=20PLPEG570DANTA; 0416=20PLPEG1055DANTA; 0421a=20PLPEG1055DAPEI4NTAZn; 0421b=20PLPEG1055DAPEI8NTA; 0603 c=20PLPEG550DAPEI4NTA-Zn; 0604c=20PLPEG550DAPEI8NTAZn; 0605c=20PLPEG550DAPEI12NTAZn; and 0804b=18PEIPEG1030DANTAZn. 1.25 ug/ml carrier with 0.25 ug/ml lysostaphin (L9043-5 mg; Sigma) in 0.1M MOPS with 1% Tween and 0.5 mM EDTA, pH 7.3, with and without Protease Inhibitor (PI) (Calbiochem; cat#539131), and with (25%) and without normal rat serum (MP Biomedicals; #642941). This is considered 20% loading where lysostaphin represents 20% of the carrier weight. The lysostaphin activity was monitored using a Bioscan plate reader.

TABLE 4
Carriers enhances metallopeptidase (lysostaphin) activity (nU +/− SD)

| | Lyso + | Lyso + | Lyso + | Lyso + | Lyso + | Lyso + | Lyso + | Lyso + | Lyso + | Lyso |
|---|---|---|---|---|---|---|---|---|---|---|
| | 0124a | 0326 | 0416 | 0421a | 0421b | 0603c | 0604c | 0605c | 0804b | Alone |
| with PI | 8150 ± | 11318 ± | 11882 ± | 9131 ± | 9922 ± | 9921 ± | 8384 ± | 7272 ± | 7271 ± | 4297 ± |
| w/o serum | 182 | 553 | 588 | 493 | 454 | 666 | 392 | 313 | 223 | 254 |
| w/o PI | 6985 ± | 11876 ± | 11665 ± | 9021 ± | 10533 ± | 10170 ± | 8283 ± | 6229 ± | 6796 ± | 3379 ± |
| w/o ser | 670 | 1210 | 1455 | 1050 | 561 | 281 | 465 | 295 | 963 | 846 |
| With PI | 5541 ± | 5773 ± | 4797 ± | 5735 ± | 4657 ± | 5440 ± | 5552 ± | 5450 ± | 2567 ± | 1323 ± |
| with serum | 469 | 96 | 89 | 107 | 72 | 100 | 174 | 240 | 90 | 176 |
| w/o PI | 5940 ± | 5793 ± | 4871 ± | 5929 ± | 4843 ± | 5499 ± | 5642 ± | 5689 ± | 2849 ± | 1542 ± |
| with ser | 133 | 77 | 202 | 290 | 131 | 718 | 131 | 309 | 221 | 115 |

### Example 23

### Carriers Preserve the Enhanced Activity of Metallopeptidase (Lysostaphin) in 25% Serum over 24 Hours Compared to the Metallopeptidase Alone

Table 5 shows carriers loaded with 20% lysostaphin. The carrier name structures are as follows: 0124a=40PLPEG539DANTA; 0326=20PLPEG570DANTA; 0416=20PLPEG1055DANTA; 0421a=20PLPEG1055DAPEI4NTAZn; 0421b=20PLPEG1055DAPEI8NTA; 0603c=20PLPEG550DAPEI4NTAZn; 0604c=20PLPEG55 ODAPEI8NTAZn; 0605c=20PLPEG550DAPEI12NTAZn; and 0804b=18PEI-PEG1030DANTAZn. Appropriate amount of ZnCl was added to those structures without Zinc prior to loading lysostaphin. For this study, the binding mixture of 400 ul in sextuplet (n=6) contains 1.25 ug/ml carrier with 0.25 ug/ml lysostaphin (L9043-5 mg; Sigma) in 0.1M MOPS with 1% Tween and 0.5 mM EDTA, pH 7.3, and with normal rat serum (25%) (MP Biomedicals; #642941). This is considered 20% loading where lysostaphin represent 20% of the carrier weight. After reading first half of mixture (200 ul), the binding mixture was stored at room temperature. Fluorescence (Ex485 nm/Em535 nm) increase was monitored over 90 min using Chameleon 96-well microplate fluorometer (Bioscan) every 7.5 minutes. The slope or the rate of increase in fluorescence per minute was determined by regression and converted to nUnit (slope×12) of enzyme activity.

TABLE 5
Carriers Preserve the enhanced activity of lysostaphin
in 25% serum compared to control over 24 hours

| | Lyso + | Lyso + | Lyso + | Lyso + | Lyso + | Lyso + | Lyso + | Lyso + | Lyso + | Lyso |
|---|---|---|---|---|---|---|---|---|---|---|
| | 0124a | 0326 | 0416 | 0421a | 0421b | 0603c | 0604c | 0605c | 0804b | alone |
| day 1 | 5483 ± | 5230 ± | 5271 ± | 5716 ± | 5099 ± | 6018 ± | 6171 ± | 5963 ± | 3715 ± | 1522 ± |
| | 175.5 | 132.5 | 97.3 | 164 | 114 | 210 | 157 | 66.4 | 208 | 39 |
| day 2 | 2751 ± | 2892 ± | 3001 ± | 2880 ± | 2678 ± | 3110 ± | 2888 ± | 2793 ± | 1976 ± | 866 ± |
| | 106.3 | 30.5 | 177 | 124 | 54 | 172 | 127 | 30 | 28.1 | 18.5 |

### Example 24

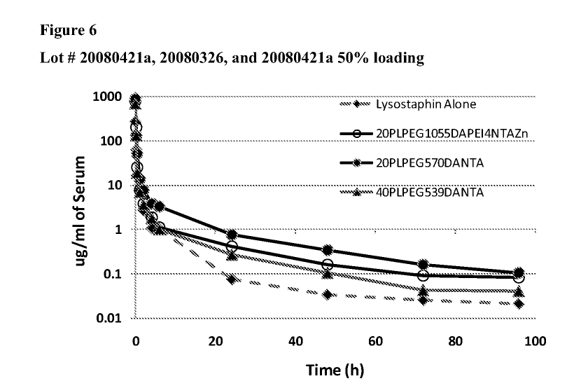

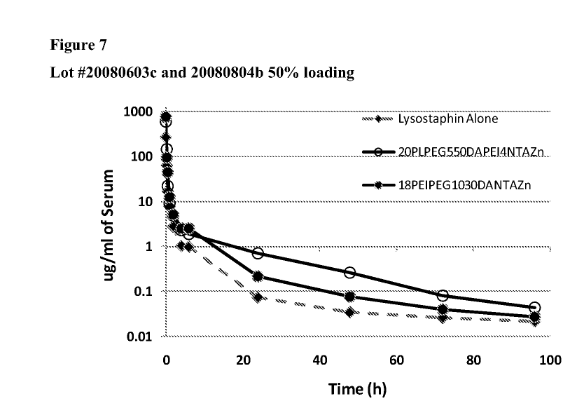

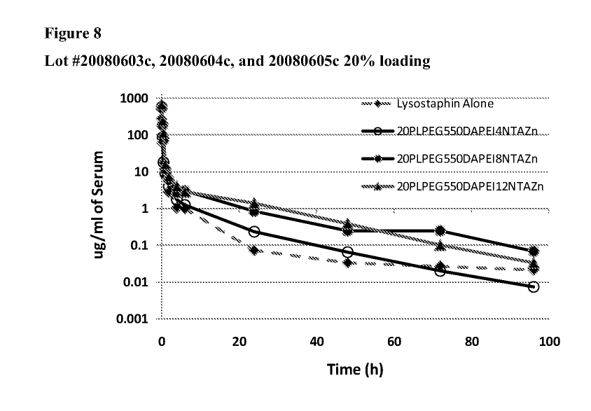

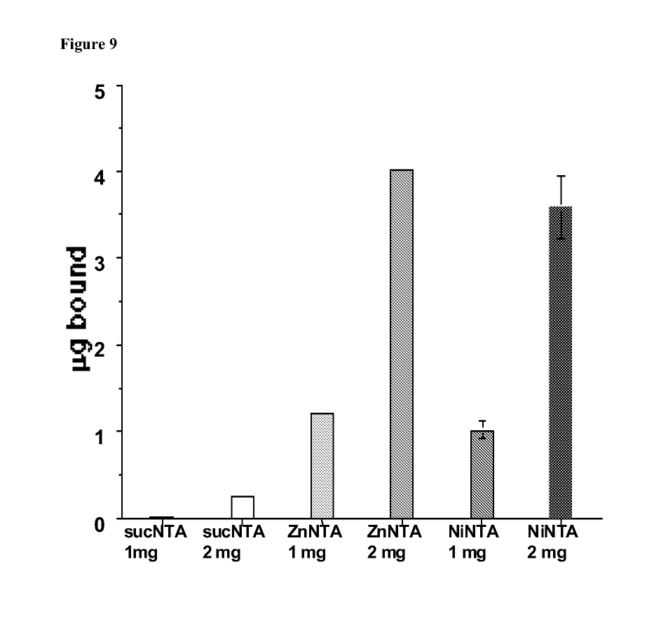

### A Metallopeptidase (Lysostaphin) Formulated in the Carrier of the Present Invention Shows Longer Blood Circulation Time than Unformulated Metallopeptidase (See FIGS. 6-8)

Blood metallopeptidase activity was measured after intravenous administration of 10 mg/kg lysostaphin in Sprague Dawley rats (n=5). The lysostaphin (AMBI; Lawrence, N.Y.; lot#GDV2) used here was formulated in carrier 20PLPEG550DAPEI4NTAZn (lot#20080603c, see Table 3 above) and carrier 20PLPEGDA570DANTA (lot#20080326, see above) at 20 or 50% loading. As can be seen in this preliminary data, the metal bridge carrier of the present invention has the ability to prolong the blood circulation half-life of lysostaphin. The noise or standard deviation of the blank in this assay is 28 nU making a reasonable detection limit of about 100-150 nU. For data points below 150 nU, the samples were re-assayed at a larger serum volume to determine the activity. The upper limit of detection for this assay is 12000 nU. Therefore, for those data points exceeding the upper detection limit of 12000 nU, samples were re-assayed at a higher dilution to get accurate detection of activity.

### Example 25

### Methods for the Determination of the Metallopeptidase (Lysostaphin) Activity

Metallopeptidase activity can also be determined using a colorimetric assay in 96-well plate format which includes TNBS titration of lysostaphin-mediated hydrolysis of N-acetylpentaglycine. N-acetylpentaglycine (NAPG) can be obtained from any peptide synthesis contract providers. Hexaglycine, once made can be modified by using acetic anhydride acetylation in the presence of TEA and this process is known to those skilled in the art. To assay for lysostaphin using NAPG, aliquots containing lysostaphin can be serially diluted in 96-well plates that contain 0-25 μg/ml in a volume of 50 μl/well. To these solutions 50 ul of 10 mM solution of NAPG can be added and plate incubated for 30 min at RT. Ten ul of 10 mM TNBS and 10 ul of 0.1 M borate, pH 9.2 can be added and the reaction can be stopped in 30 min with the addition of 1M sodium acetate, pH 4.5. Absorbance can be read at 405 nm using a plate reader (Molecular Devices). The above method allows one to determine lysostaphin at concentrations in the range of 1-25 μg/ml. Alternatively, activity can be measured by evaluating the lysis of heat-killed S. aureus by monitoring decreasing absorbance or, alternatively, by measuring viability (decrease in colony-forming units (CFUs) of live S. aureus in the presence of lysostaphin.

### Example 26

### Binding of a Model Protein (Rhgh) to PLPEGNTAZn/Ni is Dependent on the Presence of Chelated Metal

This example is presented to show that the chelator attached to the backbone is necessary for the binding of the protein with the metal binding domain. Metals such as Zinc or Nickel can be used but is not intended to limit the scope of this invention to these metals. In this particular experiment, 500 μg rhGH were mixed with 40 μl radioactively labeled trace amounts of ¹²⁵I-rhGH (concentration-5 mg/ml). Centricon YM100 was used to remove rhGH aggregates (flow-through collected). Final [rhGH]=3.22 mg/ml. Various amounts of PLPEGNTAZn/Ni were incubated with 20 μg rhGH in a volume of 100 Unbound rhGH was removed on Centricon YM100. Membrane-retained rhGH-PLPEGNTAZn/Ni complex was washed with 100 μl PBS by centrifugation. Radioactivity in eluate and retentate were determined separately using a gamma counter (Table 6): Because all metallopeptidase contain metal, they are all expected to bind to the carrier of the present invention and no undue experimentation is needed to practice the present invention with any metallopeptidase.

TABLE 6
Binding of a metal binding protein (rhGH) to the carrier
is dependent on the presence of chelated metal

| Sample, chelate attached to PLPEGSA | The fraction of rGH retained on | | μg bound minus |
|---|---|---|---|
| and carrier amount | YM100 membrane | μg bound | background |
| Membrane control | 0.05 | 1.03 | control |
| PLPEGSANi, 1 mg (lot#20020102) | 0.05 | 1.04 | 0.01 |
| PLPEGSANi, 2 mg (lot#20020102) | 0.06 | 1.29 | 0.25 |
| PLPEGNTAZn, 1 mg (lot#20020105) | 0.11 | 2.26 | 1.22 |
| PLPEGNTAZn, 2 mg (lot#20020105) | 0.25 | 5.05 | 4.02 |
| PLPEGNTANi, 1 mg (lot#20020104) | 0.10 | 2.05 | 1.01 |
| PLPEGNTANi, 2 mg (lot#20020104) | 0.23 | 4.63 | 3.60 |

Non-specific binding to YM100 membrane surface and binding to succinylated control (compound I of Example 1) polymers were similar.
Ni and Zn complexes of PLPEGNTA showed 12 to 20-fold higher binding (2 mg polymer in the incubation mixture):

### Example 27

### Size-Exclusion Analysis of a Model Protein (Rhgh) Complexed With PLPEGNTAZn

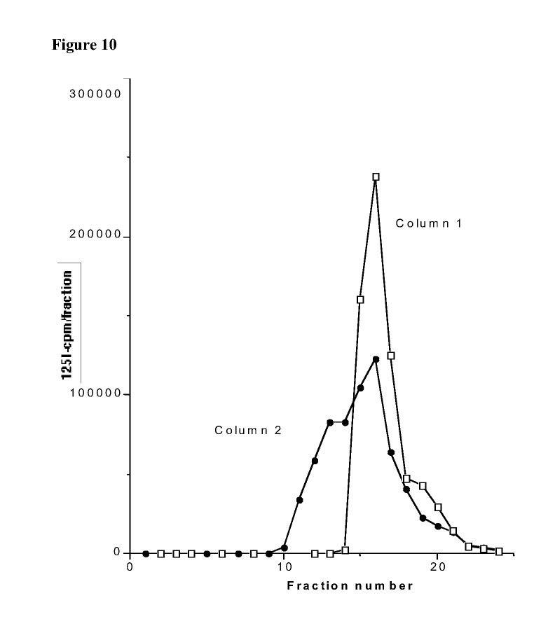

PLPEGNTAZn (100 μl, 2 mg) was mixed with 100 μg rhGH and analyzed on size-exclusion HPLC column (SEC-5, Rainin). Fractions were collected and counted using a gamma-counter (FIG. 10). The formation of a complex between the co-polymer and rhGH is evident from a change in elution pattern (fractions 11-14 contain higher molecular weight complex). The result demonstrates (FIG. 10) that the interaction of chelated metal the metal binding domain of the protein is stable and can survive the gel permeation chromatography involving thousands of re-equilibration (equal to the number of theoretical plates of the column) as the sample passes through the column. Weak interaction will cause the complex to dissociate resulting in unaltered rhGH peak which is not observed in this case.

### Example 28

### Construction of a His-Tagged Green Fluorescent Protein (GFP) Variant

A cDNA encoding for humanized GFP isoform was excised from BlueScriptGFP vector using compatible restriction sites. GFP fragment was then subcloned into SalI-KpnI-restricted pHAT10 vector (Clontech) to afford in-frame expression with His-tag (HAT™) from chicken lactate dehydrogenase (KNHLIHRVHKDDHAHAHRK) containing six histidines. Subcloning was performed by ligating the purified GFP fragment with linearized pHAT10 vector using T4 DNA ligase. Ligation reactions were used for E. coli transformation. Several colonies exhibiting bright green fluorescence under the UV light were selected. Bacterial colonies were transferred into LB broth and grown overnight in a volume of 5 ml. This starter culture was then used for infecting 1 l of LB medium grown to the density of 0.8 at 600 nm and bacterial culture was centrifuged at 6000 g to isolate bacterial mass. Bacteria were then lysed using B-PER buffer (Pierce) in the presence of 1× protease inhibitors (with no EDTA, Roche Biochemicals). Lysate was cleared by centrifugation at 16000×g (SS-34 Rotor, Sorvall) and the supernatant was combined with washed, pre-equilibrated TALON™ resin (Clontech). The mixture was agitated at 4 C overnight and washed several times with loading buffer (50 mM phosphate, 300 mM NaCl pH 7). Histidine tagged-GFP product was eluted using 100 mM imidazole in 45 mM Na-phosphate, 270 mM NaCl, pH 7). Fluorescent eluate was dialyzed against PBS, pH 7 and analyzed by electrophoresis.

### Example 29

### Binding of Histidine Tagged-GFP to PLPEGNTA and Control Polymers (see Table 7

This example is presented here to demonstrate that a protein can be modified with a chelating molecule such as a histidine tag to allow it to bind or enhance its binding to carriers of the present invention. Similar process can be performed with metalloendopeptidases of the present invention. Complex formation between PLPEGNTA copolymer and histidine-tagged GFP was achieved by combining histidine tagged-GFP and Ni²⁺ or Zn²⁺ salts of PLPEGNTA or PLPEGSA (control). After an hour of incubation, the complexes were placed in YM-50 membrane. Various amounts of PLPEGNTAZn/Ni were incubated with 20 μg histidine tagged-GFP in a volume of 100 μl. Free non-bound histidine tagged-GFP was removed on Centricon YM100. Membrane-retained PLPEGNTAZn/Ni complex was washed three times using 100 μl PBS aliquots by centrifugation. The fluorescence intensities in eluate and retentate were determined using a fluorometer (excitation 475, emission 510 nm). In some experiments, 100% mouse plasma was added to the incubation mixtures and samples were processed as described before.

TABLE 7
Proteins can be modified with histidine to bind or to improve
the binding to the metal chelated containing carrier.

| Sample | % GFP bound |
|---|---|
| GFP control | 0.002 |
| PLPEGSA Zn control | 0.003 |
| PLPEGNTAZn | 99.68 |
| PLPEGNTANi | 99.52 |

The obtained result shows that the binding of histidine tagged-GFP to metal chelate of PLPEGNTAZn/Ni co-polymer was highly specific (Table 8) and that the association of H isTagged-GFP with a similar co-polymer bearing no NTA residues was close to the background.

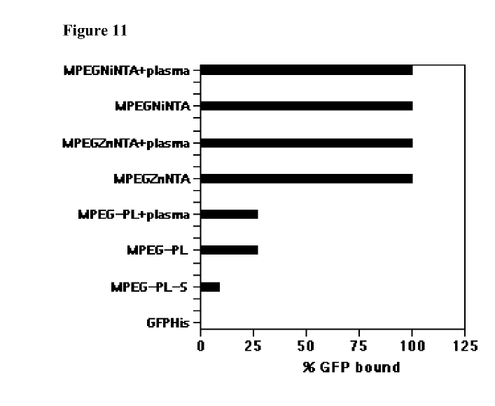

In the presence of plasma binding of histidine tagged-GFP was also highly specific. Binding to NTA-linked co-polymers in the presence of Ni and Zn cations was approximately the same in the presence or in the absence of the plasma. The only detectable non-specific binding levels were detectable in the case of polycationic PLPEG co-polymer (FIG. 11) and this binding was not inhibited by plasma.

### Example 30

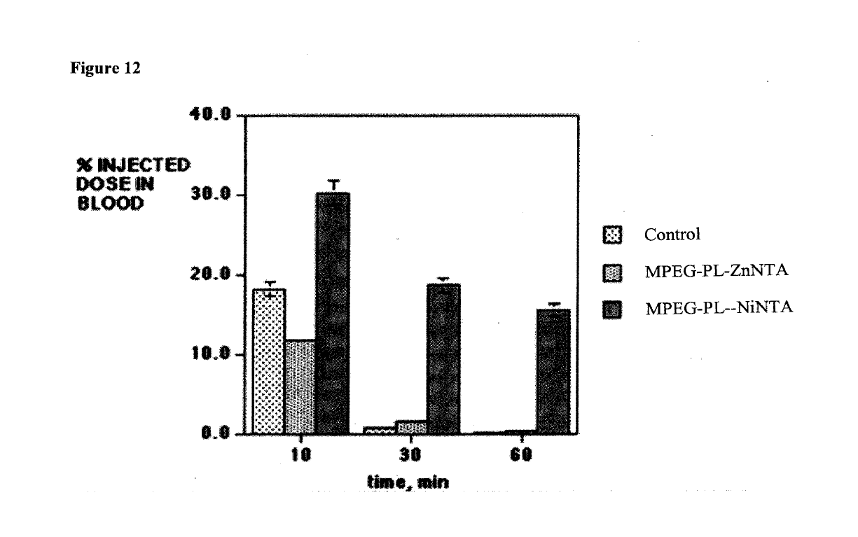

### Distribution of Histidine Tagged-GFP and Histidine Tagged-GFP-PLPEGNTAZn/Ni Complexes in Vivo after Intravenous Injection (see FIG. 12)

This example is presented to demonstrate that if lysostaphin is tagged with histidine, the distribution in the blood with time is expected to be similar to this surrogate protein (histidine tagged GFP) and that the presence of the carrier of the present invention will similarly improve the time of residency in the blood. This is also supported by FIGS. 6-8. For FIG. 12, pre-formed complexes of histidine tagged-GFP with PLPEGNTANi (lot#20020104) and PLPEGNTAZn (lot#20020105) as well as control histidine tagged-GFP were injected IV in the tail vein of anesthetized balb/c mice (20 μg histidine tagged-GFP mixed with 1 mg of co-polymer or 20 μg histidine tagged-GFP in a total volume of 0.1 ml, 2 per group) and blood samples were drawn through a catheter inserted in a contralateral tail vein. Blood samples (40 μl) were heparinized, centrifuged (3,000 g) and plasma samples were analyzed for histidine tagged-GFP using fluorometry (excitation-475/emission 508 nm). Observed fluorescence intensity values were normalized for injection dose using histidine tagged-GFP standard diluted in mouse plasma. The blood volume was calculated as 7% of animal weight and hematocrit—at 50%.

### Example 31

### Formulation and Determination of Carrier:Metallopeptidase Complex Formation Efficiency

In vivo efficacy experiments with an unformulated metallopeptidase such as lysostaphin have demonstrated that a dose of 5 mg/kg t.i.d for 3 days is effective in sterilizing vegetations in endocarditis. Conservatively, assuming no improvement in half life or efficacy, a minimum of 10% loading w/w of lysostaphin to the carrier (5 mg/50 mg) to have an acceptable volume (0.5 ml) for bolus IV administration (see Table 8) is estimated. Importantly, however, it is estimated that a 50-fold lower dose of lysostaphin formulated with carrier versus unformulated lysostaphin based on an increase in its half life (anticipated to be at least 10 fold) coupled with the increased concentration (5 fold) of the enzyme at sites of infection is required. Concentrations of carrier not exceeding 100 mg/ml will be worked with since the increased viscosity of higher concentrations would preclude its administration by IV. It should be noted that in some examples above, 50 to 100% loading is possible.

TABLE 8
Target dose for bolus injection and required loading

| | Target loading | | | |
|---|---|---|---|---|
| | (mg lysostaphin/mg | lysostaphin | Carrier | lysostaphin/carrier |
| Target dose | carrier) | MW (kDa) | MW (kDa) | (mole/mole) |
| 5 mg in 0.5 mL | 5/50 (or 10% loading) | 27 | ~550 | ~2/1 |

For comparative efficacy experiments between unformulated and formulated lysostaphin it will be important to determine the loading capacity/binding efficiency of the carrier. Formulations of lysostaphin can be evaluated by complex formation during the incubation with PLPEGNTA and PLPEGNTAZn in different conditions (altering pH, temperature, ionic strength, and chelate pretreatment (to remove Zn or not of lysostaphin). The formulation with highest loading can be selected for further evaluation.

### Example 32

### Other Methods for Determination of the Efficiency of the Carrier:Metallopeptidase Complex Formation

Alternative methods to evaluate the efficiency of binding of the carrier to a metallopeptidase include the radioiodination of the metallopeptidase. Radio-iodinated metallopeptidase can be obtained by using sodium [¹²⁵I] iodide in the presence of Iodo-Gen (Pierce) at approx. 0.01-0.05 mCi/μg peptide followed by purification on C18-reversed phase HPLC column using a gradient of acetonitrile in 0.1% TFA. Due to the possibility of additional histidine radioiodination reactions in the presence and in the absence of trace amounts of Zn to protect the His residue will be performed. The ability of the peptide to form a complex with ZnNTA after the radioiodination can be tested by measuring the retention of radioactivity on Zn-saturated NTA-column. Trace amounts of radioiodinated metallopeptidase can be mixed with cold metallopeptidase followed by the incubation with Carrier-Zn (PLPEGNTAZn) or to determine complex formation efficiency. Additionally, considering the metallopeptidase such as lysostaphin, as purchased, likely has a zinc already present in its active site (AMBI, personal communication), labeled and unlabelled metallopeptidase can also be mixed with carrier without zinc already chelated to the PLPEGNTA. Unbound metallopeptidase can be removed using Microcon YM100-ultrafiltration followed by the separate radioactivity determination in the eluate and the retentate.

### Example 33

### Measurement of Anti-lysostaphin-Binding Activity of Formulated Metallopeptidase (Lysostaphin) Versus Free Lysostaphin

Lysostaphin, a microbial protein product, is immunogenic and repeated administration can generate an immune response. It is expected that lysostaphin associated with carriers of the present invention is protected from binding to antibodies and this can be evaluated by binding to immobilized anti-lysostaphin antibodies in an Enzyme Linked Immunosorbent assay (ELISA). The complex of Carrier and ¹²⁵I-lysostaphin with known specific radioactivity can be incubated with anti-lysostaphin polyclonal affinity-purified antibodies immobilized on the surface of a flexible 96well immunoplate (Nunc). In positive control experiments, ¹²⁵I-lysostaphin alone can be used. The binding of lysostaphin and its complex with the carrier can be compared to: 1) 121-lysostaphin binding to the plate in the presence of the excess of the antibody; 2) ¹²⁵I-lysostaphin binding to the plate in the presence of free succinylated carrier. To determine binding, wells can be cut out and counted in a gamma-counter separately.

### Example 34

### Loading Neprilysin to the Carrier to Make a Composition for the Treatment of Alzheimer's Disease

Neprilysin, interchangeably known as neutral endopeptidase (NEP), CD10, and common acute lymphoblastic leukemia antigen (CALLA), is a zinc-dependent metallopeptidase enzyme that degrades a number of small secreted peptides, most notably the amyloid beta peptide whose abnormal misfolding and aggregation in neural tissue has been implicated as a cause of Alzheimer's disease. Synthesized as a membrane-bound protein, the neprilysin ectodomain is released into the extracellular domain after it has been transported from the Golgi apparatus to the cell surface. Because neprilysin is a metallopeptidase, it can bind to the carriers of the present invention and is demonstrated as follows. About two hundred fifty mg of any of the metal bridge carriers described herein, and in sections above, are mixed with about 2.5 mg to about 50 mg of neprilysin in appropriate buffer (about 10-50 mM HEPES buffer, about pH 7.3 will be ideal but any buffer between about pH 5-8 can be used). The binding can be evaluated as in Examples 20-21.

To make a pharmaceutical composition for the treatment of Alzheimer's disease, about 250 mg of any of the metal bridge carriers described herein are be mixed with between about 2.5 mg to about 50 mg of neprilysin. The method of treatment of Alzheimer's disease comprises dissolving the above pharmaceutical composition in an appropriate buffer and/or excipient, and injecting (preferably subcutaneously) into a patient diagnosed with, or suspected of having or developing Alzheimer's disease. This is expected to result in a slow and sustainable (over many hours) release of neprilysin from the carrier into the blood and the released neprilysin can degrade the circulating Abeta amyloid in the blood facilitating, its turnover. As the blood circulating Abeta amyloid degrades, more Abeta amyloid moves from the brain to the blood facilitating clearance of Abeta amyloid from the brain.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

1-64. (canceled)

65. A composition comprising:
a polymer backbone comprising monomeric units;
a protective chain covalently bonded to a monomeric unit in the polymer backbone;
a chelating group covalently bonded to a monomeric unit in the polymer backbone;
a transition metal ion chelated to the chelating group; and
a metallopeptidase coordinately bonded to the transition metal ion.

66. The composition of claim 65, wherein the protective side chain comprises poly(ethyleneglycol) and has a molecular weight of between 2,000 to 20,000 Daltons.

67. The composition of claim 66, wherein the polymer backbone is a polyamino acid.

68. The composition of claim 67, wherein the polyamino acid is selected from polylysine, polyornithine, polyarginine, polyglutamate, polyaspartate, polyserine, polythreonine, and polytyrosine.

69. The composition of claim 66, wherein the polymer backbone is a polysaccharide.

70. The composition of claim 66, wherein the polymer backbone is selected from the group consisting of branched or unbranched polyethyleneimine, branched or unbranched polyallylamine, branched or unbranched polyamidoamine, branched or unbranched polyacrylic acid, and branched or unbranched polyvinylalcohol.

71. The composition of claim 65, wherein the chelating group comprises one or more of the following:
1,4,7,10-tetraazacyclododecane-N,N′,N″,N′″-tetraacetic acid;
1,4,7,10-tetraaza-cyclododecane-N,N′,N″-triacetic acid;
1,4,7-tris(carboxymethyl)-10-(2′-hydroxypropyl)-1,4,7,10-tetraazocyclodecane;
1,4,7-triazacyclonane-N,N′,N″-triacetic acid;
1,4,8,11-tetraazacyclotetra-decane-N,N′,N″,N′″-tetraacetic acid;
1,2-diaminocyclohexane-N,N,N′,N′-tetraacetic acid;
bis(aminoethanethiol)carboxylic acid;
diethylenetriamine-pentaacetic acid (DTPA);
ethylenediamine-tetraacetic acid (EDTA);
ethyleneglycoltetraacetic acid (EGTA);
ethylene-bis(oxyethylene-nitrilo)tetraacetic acid;
ethylenedicysteine;
Imidodiacetic acid (IDA);
N-(hydroxyethyl)ethylenediaminetriacetic acid;
nitrilotriacetic acid (NTA);
nitrilodiacetic acid (NDA);
triethylenetetraamine-hexaacetic acid (TTHA);
trimethyl-1,4,7-triazacyclononane(TACN); or
a peptide having the formula: (AₓH_{y})ₚ, wherein A is any amino acid residue, H is histidine, x is an integer from 0-6; y is an integer from 1-6; and p is an integer from 2-6.

72. The composition of claim 71, wherein the transition metal ion is one or more
of the following: Zn²⁺, Ni²⁺, Co²⁺, Fe²⁺, Mn²⁺, or Cu²⁺.

73. The composition of claim 67, wherein the polyamino acid comprises polylysine, the chelating group comprises NTA, the metal ion is zinc, and the metallopeptidase is lysostaphin.

74. The composition of claim 73, further comprising an antibiotic selected from: amoxicillin, ampicillin, azidocillin, azlocillin, aztreonam, bacitacin, benzathine benzylpenicillin, benzathine phenoxymethylpenicillin, benzylpenicillin(G), biapenem, carbenicillin, cefacetrile, cefadroxil, cefalexin, cefaloglycin, cefalonium, cefaloridine, cefalotin, cefapirin, cefatrizine, cefazedone, cefazaflur, cefazolin, cefradine, cefroxadine, ceftezole, cefaclor, cefamandole, cefminox, cefonicid, ceforanide, cefotiam, cefprozil, cefbuperazone, cefuroxime, cefuzonam, cephamycin (such as cefoxitin, cefotetan, cefmetazole), carbacephem (such as loracarbef), cefcapene, cefdaloxime, cefdinir, cefditoren, cefetamet, cefixime, cefinenoxime, cefodizime, cefoperazone, cefotaxime, cefpimizole, cefpiramide, cefpodoxime, cefsulodin, ceftazidime, cefteram, ceftibuten, ceftiolene, ceftizoxime, ceftriaxone, oxacephem (such as flomoxef, latamoxef), cefepime, cefozopran, cefpirome, cefquinome, ceftobiprole, chloroamphenicol, chlorohexidine, clindamycin, clometocillin, cloxacillin, colistin, cycloserine, daptomycin, doripenem, doxycycline, epicillin, ertapenem, erythromycin, faropenem, fostomycin, gentamycin, imipenem, linezolid, mecillinam, meropenem, methicillin, meticillin, mezlocillin, minocycline, mupirocin, nafcillin, neomycin, oxacillin, panipenem, penamecillin, pheneticillin, phenoxymethylpenicillin (V), piperacillin, polymyxin, polymyxin B, procaine benzylpenicillin, propicillin, quinupristin/dalfopristin, ramoplanin, rifampicin, rifampin, sulbenicillin, teicoplanin, tigecycline, tigemonam, trimethoprim/sulfamethoxazole, and vancomycin.

75. The composition of claim 67, wherein the polyamino acid comprises polylysine, the chelating group comprises NTA, the metal ion is zinc, and the metallopeptidase is neprilysin.

76. A composition comprising:
an aliphatic chain backbone;
a poly(ethyleneglycol) covalently bonded to aliphatic chain;
a chelating group covalently bonded to the aliphatic chain;
a metal ion chelated to the chelating group; and
a metallopeptidase coordinately bonded to the metal ion.

77. The composition of claim 76, wherein the aliphatic chain comprises from C8 to C36 carbon atoms inclusive.

78. The composition of claim 77, wherein the chelating group comprises one or more of the following:
1,4,7,10-tetraazacyclododecane-N,N′,N″,N′″-tetraacetic acid;
1,4,7,10-tetraaza-cyclododecane-N,N′,N″-triacetic acid;
1,4,7-tris(carboxymethyl)-10-(2′-hydroxypropyl)-1,4,7,10-tetraazocyclodecane;
1,4,7-triazacyclonane-N,N′,N″-triacetic acid;
1,4,8,11-tetraazacyclotetra-decane-N,N′,N″,N′″-tetraacetic acid;
1,2-diaminocyclohexane-N,N,N′,N′-tetraacetic acid;
bis(aminoethanethiol)carboxylic acid;
diethylenetriamine-pentaacetic acid (DTPA);
ethylenediamine-tetraacetic acid (EDTA);
ethyleneglycoltetraacetic acid (EGTA);
ethylene-bis(oxyethylene-nitrilo)tetraacetic acid;
ethylenedicysteine;
Imidodiacetic acid (IDA);
N-(hydroxyethyl)ethylenediaminetriacetic acid;
nitrilotriacetic acid (NTA);
nitrilodiacetic acid (NDA);
triethylenetetraamine-hexaacetic acid (TTHA);
Trimethyl-1,4,7-triazacyclononane(TACN); or
a peptide having the formula: (AₓH_{y})ₚ, wherein A is any amino acid residue, H is histidine, x is an integer from 0-6; y is an integer from 1-6; and p is an integer from 2-6.

79. The composition of claim 78, wherein the transition metal ion is one or more of the following: Zn²⁺, Ni²⁺, CO²⁺, Fe²⁺, Mn²⁺, or Cu²⁺.

80. The composition of claim 77, wherein the chelating group is NTA, the metal ion is Zn²⁺, and the metallopeptidase is lysostaphin.

81. The composition of claim 80, further comprising an antibiotic selected from: amoxicillin, ampicillin, azidocillin, azlocillin, aztreonam, bacitacin, benzathine benzylpenicillin, benzathine phenoxymethylpenicillin, benzylpenicillin(G), biapenem, carbenicillin, cefacetrile, cefadroxil, cefalexin, cefaloglycin, cefalonium, cefaloridine, cefalotin, cefapirin, cefatrizine, cefazedone, cefazaflur, cefazolin, cefradine, cefroxadine, ceftezole, cefaclor, cefamandole, cefminox, cefonicid, ceforanide, cefotiam, cefprozil, cefbuperazone, cefuroxime, cefuzonam, cephamycin (such as cefoxitin, cefotetan, cefmetazole), carbacephem (such as loracarbef), cefcapene, cefdaloxime, cefdinir, cefditoren, cefetamet, cefixime, cefmenoxime, cefodizime, cefoperazone, cefotaxime, cefpimizole, cefpiramide, cefpodoxime, cefsulodin, ceftazidime, cefteram, ceftibuten, ceftiolene, ceftizoxime, ceftriaxone, oxacephem (such as flomoxef, latamoxef), cefepime, cefozopran, cefpirome, cefquinome, ceftobiprole, chloroamphenicol, chlorohexidine, clindamycin, clometocillin, cloxacillin, colistin, cycloserine, daptomycin, doripenem, doxycycline, epicillin, ertapenem, erythromycin, faropenem, fostomycin, gentamycin, imipenem, linezolid, mecillinam, meropenem, methicillin, meticillin, mezlocillin, minocycline, mupirocin, nafcillin, neomycin, oxacillin, panipenem, penamecillin, pheneticillin, phenoxymethylpenicillin (V), piperacillin, polymyxin, polymyxin B, procaine benzylpenicillin, propicillin, quinupristin/dalfopristin, ramoplanin, rifampicin, rifampin, sulbenicillin, teicoplanin, tigecycline, tigemonam, trimethoprim/sulfamethoxazole, and vancomycin.

82. The composition of claim 77, wherein the chelating group is NTA, the metal ion is Zn²⁺, and the metallopeptidase is neprilysin.

83. A method of treating an infection in a subject in need of such treatment comprising administering to the subject an effective amount of the composition comprising any one of claim 73 or 80.

84. A method of treating a subject diagnosed with, or suspected of having or developing Alzheimer's disease comprising administering to the subject an effective amount of the pharmaceutical composition comprising anyone of claim 75 or 82.
